(19) United States
(12) Patent Application Publication
(10) Pub. No.: US 2023/0218422 A1
(43) Pub. Date: Jul. 13, 2023

(54) ASSEMBLY FOR HELPING TO RESTRAIN A SUBJECT

(71) Applicant: Varoujan Kostanian, Las Vegas, NV (US)

(72) Inventor: Varoujan Kostanian, Las Vegas, NV (US)

(21) Appl. No.: 18/096,304

(22) Filed: Jan. 12, 2023

Related U.S. Application Data
(60) Provisional application No. 63/298,744, filed on Jan. 12, 2022.

(51) Int. Cl. A61F 5/37

(52) U.S. Cl. CPC A61F 5/3707

(57) ABSTRACT

A head restraint assembly includes a restraint platform configured to receive a head of a subject. First and second head restraints are attached to the restraint platform and configured to selectively engage first and second sides of the subject's head. The first head restraint has a first cheekbone flange configured to selectively engage a cheekbone region of the subject's head on the first side of the subject's head. The second head restraint has a second cheekbone flange configured to selectively engage a cheekbone region of the subject's head on the second side of the subject's head. A cheekbone strap extends between the first and second cheekbone flanges and is configured for selectively tightening the cheekbone flanges against the subject's cheekbone regions. The cheekbone strap is configured to be spaced from the subject's head when the subject's head is restrained by the head restraint assembly.

### RELATED APPLICATION

This application claims priority from U.S. Provisional Application No. 63/298,744, filed 12 Jan. 2022, the subject matter of which is incorporated herein by reference in its entirety for all purposes.

### TECHNICAL FIELD

This disclosure generally relates to a head restraint assembly, a body stabilizer assembly, and a restraint assembly for helping to restrain a subject. More particularly, this disclosure relates to a head restraint assembly, an abdominal sling, a body stabilizer assembly, and a restraint assembly that includes one or more of a head restraint assembly, an abdominal sling, and a body stabilizer.

### BACKGROUND

Restraint assemblies for restraining at least a portion of a subject are known. As used herein, “restraint” encompasses immobilizing at least a portion of a subject's body for any reason, including, but not limited to, protecting the subject from others, protecting others from the subject, and/or preventing potentially deleterious motion of a portion of the subject's body that may be injured. Certain known restraint assemblies provide challenges to medical professionals for several reasons. For example, certain known restraint assemblies are not adaptable to the subject's anatomy, only restrain a portion of a subject, are not adapted to pull portions of a subject's abdominal wall soft tissue cephalad, and/or are not capable of being used in a magnetic resonance imaging (“MRI”) scanner.

### SUMMARY

In an aspect, alone or in combination with any other aspect, a head restraint assembly comprises a restraint platform configured to receive a head of a subject. A first head restraint is attached to the restraint platform and configured to selectively engage a first side of the subject's head. The first head restraint has a first cheekbone flange configured to selectively engage a cheekbone region of the subject's head on the first side of the subject's head. A second head restraint is attached to the restraint platform and configured to selectively engage a second side of the subject's head. The second head restraint has a second cheekbone flange configured to selectively engage a cheekbone region of the subject's head on the second side of the subject's head. A cheekbone strap extends between the first and second cheekbone flanges and is configured for selectively tightening the cheekbone flanges against the subject's cheekbone regions. The cheekbone strap is configured to be spaced from the subject's head when the subject's head is restrained by the head restraint assembly.

In an aspect, alone or in combination with any other aspect, a head restraint assembly comprises a restraint platform configured to receive a head of a subject. A first head restraint is attached to the restraint platform and configured to selectively engage a first side of the subject's head. A second head restraint is attached to the restraint platform and configured to selectively engage a second side of the subject's head. A first articulating assembly has a first portion attached to the first head restraint and a second portion connected to the restraint platform. The first portion of the first articulating assembly is selectively articulatable relative to the second portion of the first articulating assembly and thereby provides the first head restraint with at least three degrees of freedom of movement for the first head restraint to conform to the first side of the subject's head. A second articulating assembly has a first portion attached to the second head restraint and a second portion connected to the restraint platform. The first portion of the second articulating assembly is selectively articulatable relative to the second portion of the second articulating assembly and thereby provides the second head restraint with at least three degrees of freedom of movement for the second head restraint to conform to the second side of the subject's head.

In an aspect, alone or in combination with any other aspect, an abdominal sling comprises a central sling portion configured to selectively engage the subject's abdominal wall skin. At least one sling strap extends from the central sling portion and configured to be selectively tensioned. The abdominal sling is configured such that, when the central device portion engages the subject's abdominal wall skin and the at least one sling strap is tensioned, the central sling portion exerts a force on the subject's abdominal wall skin to urge the subject's abdominal wall soft tissue cephalad.

In an aspect, alone or in combination with any other aspect, a body stabilizer assembly for helping to restrain a subject comprises a body stabilizer that is configured to receive a portion of the subject. The body stabilizer includes at least one stabilizer aperture longitudinally positioned along a first lateral stabilizer side of the body stabilizer and at least one stabilizer aperture longitudinally positioned along an opposite second lateral stabilizer side of the body stabilizer. Each stabilizer aperture extends transversely through the body stabilizer. The body stabilizer assembly comprises a plurality of strap connectors. Each strap connector is at least partially positioned in an associated stabilizer aperture and selectively longitudinally moveable along the associated stabilizer aperture. At least one restraining strap is configured to be selectively attached to the body stabilizer via the strap connectors and selectively prevent the egress of the subject from the body stabilizer. Each end of the at least one restraining strap is selectively connected to an associated strap connector. Via the strap connectors, the at least one restraining strap is selectively longitudinally moveable relative to the body stabilizer to correspond to the subject's anatomy.

In an aspect, alone or in combination with any other aspect, a restraint assembly for helping to restrain a subject comprises at least one of a head restraint assembly that is configured to receive at least a subject's head, a body stabilizer assembly having a body stabilizer that is configured to receive a portion of a subject's body, a foot platform that is configured to receive a portion of a subject's legs and/or a portion of the subject's feet, and an abdominal sling that is configured to exert a force on a subject's abdominal wall skin to urge the subject's abdominal wall soft tissue cephalad.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a top view of a head restraint assembly according to an an aspect of the present disclosure;

FIG. 2 is a perspective side view of the aspect of FIG. 1;

FIG. 3 is a perspective top view of the aspect of FIG. 1, including the head restraint assembly in an example use environment;

FIG. 4 is a perspective rear view of the aspect of FIG. 1, including the head restraint assembly in an example use environment, and including a portion of the head restraint assembly in an alternate configuration;

FIG. 5 is a top view of an element of the aspect of FIG. 1;

FIG. 6 is a perspective front view of the aspect of FIG. 4, including the head restraint assembly in an example use environment;

FIG. 7 is a top view of the aspect of FIG. 4, including the head restraint assembly in an example use environment;

FIG. 8 is a perspective top view of a restraint assembly according to an aspect of the present disclosure;

FIG. 9 is a perspective front view of a portion of the aspect of FIG. 8;

FIG. 10 is a perspective bottom view of the aspect of FIG. 8, including the restraint assembly in a preassembled state;

FIG. 11 is a perspective top view of the aspect of FIG. 8, including the restraint assembly in an example use environment;

FIG. 12 is a side view of the aspect of FIG. 8, including the restraint assembly in an example use environment;

FIG. 13 is a top view of an element of the aspect of FIG. 8;

FIG. 14 is a bottom view of the aspect of FIG. 13;

FIG. 15 is a perspective bottom view of a portion of the aspect of FIG. 1;

FIG. 16 is a perspective side view of a head restraint assembly according to an aspect of the present disclosure;

FIG. 17 is a partially exploded, perspective side view of the aspect of FIG. 16;

FIG. 18 is a perspective top view of an element of the aspect of FIG. 16;

FIG. 19 is a perspective top view of an element of the aspect of FIG. 16;

FIG. 20 is a top view of the element of FIG. 19;

FIGS. 21-27 illustrate an example sequence of disassembling the head restraint assembly of FIG. 16;

FIG. 28 is a is a perspective top view of a body stabilizer assembly according to an aspect of the present disclosure;

FIG. 29 is a perspective front view of a portion of the aspect of FIG. 28;

FIG. 30 is a perspective top view of the aspect of FIG. 28;

FIG. 31 is a perspective side view of an element of the aspect of FIG. 28, including the element in a preassembled state;

FIG. 32 is a perspective top view of the element of FIG. 31, including the element in an assembled state;

FIG. 33 is a perspective top view of the aspect of FIG. 28, including an element of FIG. 28 in various angular positions;

FIG. 34 is a perspective top view of an element of the aspect of FIG. 28;

FIG. 35 is a perspective top view of a portion of the aspect of FIG. 28;

FIG. 36 is a partially exploded, perspective top view of the aspect of FIG. 35;

FIG. 37 is a perspective top view of a restraint assembly according to an aspect of the present disclosure, including the restraint assembly in an example use environment;

FIG. 38 is a bottom view of an element of the aspect of FIG. 37;

FIG. 39 is a perspective top view of a restraint assembly according to an aspect of the present disclosure;

FIG. 40 is a partially exploded, perspective top view of a body stabilizer assembly of the restraint assembly of FIG. 39;

FIG. 41 is a top view of an element of the aspect of FIG. 39;

FIG. 42 is a perspective bottom view of the element of FIG. 41;

FIGS. 43-45 illustrate an example sequence of moving an element of the aspect of FIG. 39 from a first condition to a second condition;

FIG. 46 is a perspective side view of an element of the aspect of FIG. 39, including the element in a preassembled state; and

FIG. 47 is a top view of the body stabilizer assembly of FIG. 40, including an element of the body stabilizer assembly depicted in various angular positions.

### DESCRIPTION OF EMBODIMENTS

Unless defined otherwise, all technical and scientific terms used herein have the same meaning as is commonly understood by one of ordinary skill in the art to which the present disclosure pertains.

As used herein, the term “subject” can refer to any warm-blooded organism including, but not limited to, human beings, pigs, rats, mice, birds, cats, dogs, goats, sheep, horses, monkeys, apes, rabbits, cattle, farm animals, livestock, etc.

As used herein, the term “medical professional” can be used interchangeably to refer to an individual who prepares for, assists with, and/or performs a procedure, and/or to an individual who prepares for, assists with, and/or performs the operation of a tool.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “on,” “attached” to, etc., another element, it can be directly on, attached to or connected to the other element or intervening elements may also be present. In contrast, when an element is referred to as being, for example, “directly connected” to another element, there are no intervening elements present. It will also be appreciated by those of skill in the art that references to a structure or feature that is disposed “adjacent” another feature may not have portions that overlap or underlie the adjacent feature.

Spatially relative terms, such as “below,” “above” and the like, may be used herein for ease of description to describe one element or feature's relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “below” other elements or features would then be oriented “above” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

The invention comprises, consists of, or consists essentially of the following features, in any combination.

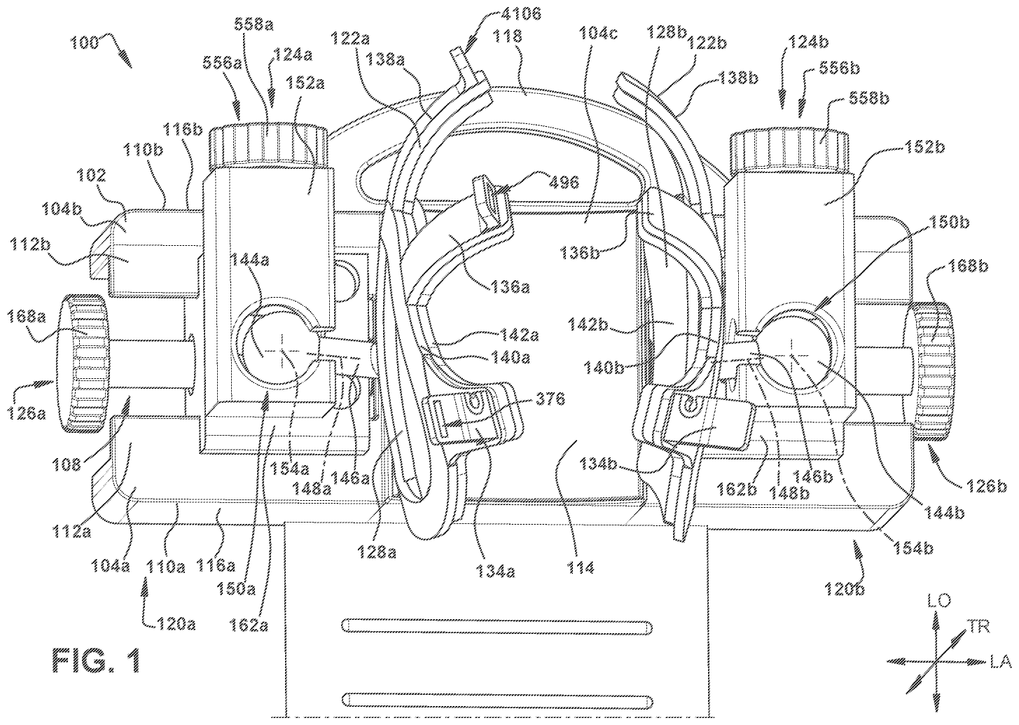

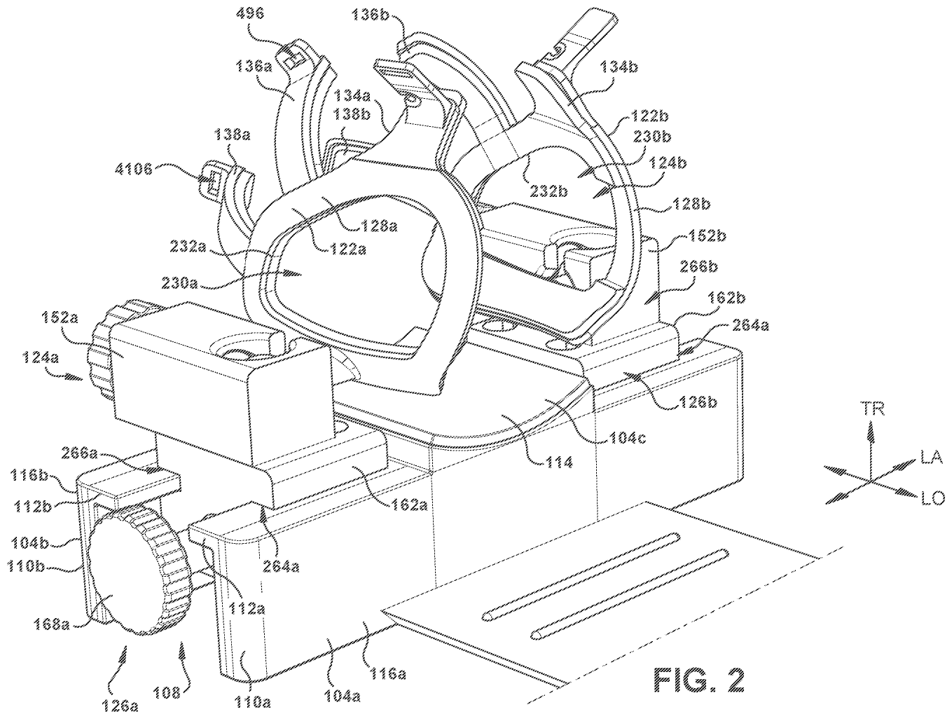

FIGS. 1-3 depict a head restraint assembly 100 for selectively restraining and/or immobilizing a subject's head H during transportation, during a medical procedure, and/or for any other desired purpose(s). The head restraint assembly 100 includes a restraint platform 102. The restraint platform 102 has a first platform portion 104a and a second platform portion 104b that is separated from the first platform portion 104a by a longitudinally extending restraint gap 108. The term “longitudinal” is used herein to indicate a substantially vertical direction, in the orientation of FIG. 1, and is indicated as “LO” in FIGS. 1-3. The first platform portion 104a includes a transversely extending first column 110a and a first flange 112a extending laterally from the first column 110a toward the second platform portion 104b. The term “transverse” is used herein to indicate a direction substantially perpendicular to the “longitudinal” direction, is shown as the vertical direction in the orientation of FIG. 2, and is indicated at “TR” in FIGS. 1-3. The term “lateral” is used herein to indicate a direction substantially perpendicular to the “longitudinal” and “transverse” directions, is shown as the horizontal direction in the orientation of FIG. 1, and is indicated at “LA” in FIGS. 1-3. The second platform portion 104b includes a longitudinally extending second column 110b and a second flange 112b extending laterally from the second column 110b toward the first platform portion 104a. The first and second flanges 112a, 112b extend toward one another and, thus, a portion of the restraint gap 108 that extends between the first and second flanges 112a, 112b may be longitudinally smaller than a portion of the restraint gap 108 that extends between the first and second columns 110a, 110b.

A third platform portion 104c of the restraint platform 102 extends longitudinally between the first and second platform portions 104a, 104b and connects the first and second platform portions 104a, 104b to one another. At least one of the first, second, and third platform portions 104a, 104b, 104c may be formed separately from and then subsequently connected to at least one other of the first, second, and third platform portions 104a, 104b, 104c. Alternatively or additionally, at least one of the first, second, and third platform portions 104a, 104b, 104c may be integrally formed with at least one other of the first, second, and third platform portions 104a, 104b, 104c as a single-piece, unitary construct.

The third platform portion 104c is configured to have the subject's head H laid thereon. As shown in FIG. 2, the third platform portion 104c may be substantially arcuate in the lateral direction in order to accommodate the subject's head H. However, it is contemplated that the third platform portion 104c may be curved in any desired manner or extend substantially flatly in the lateral direction in order to accommodate the subject's head H. The third platform portion 104c may positioned transversely on the first and second flanges 112a, 112b so that the third platform portion 104c is at least partially raised transversely above the first and second platform portions 104a, 104b. The raised third platform portion 104c may be easier for a medical professional to locate and, thus, raising the third platform portion 104c above the remaining platform portions 104a, 104b may help the medical professional position the subject's head H on the third platform portion 104c.

As shown in FIGS. 1-3, the third platform portion 104c may have a reusable or disposable base cushion 114 provided thereon that is configured to be interposed between the subject's head H and the third platform portion 104c. The base cushion 114 provides a softer surface for the subject's head H to be laid on than the substantially rigid third platform surface 104c. The base cushion 114 may be substantially permanently attached to the third platform portion 104c or may be configured to be selectively removed from the third platform portion 104c as desired.

As shown in FIGS. 1-2, the first column 110a includes a longitudinally facing first platform face 116a, while the second column 110b includes a longitudinally facing second platform face 116b that is opposite the first platform face 116a. The restraint platform 102 may include a gripping element 118 for use in carrying/transporting the restraint platform 102 and/or head restraint assembly 100 as a whole. As shown in FIG. 1, the gripping element 118 may extend longitudinally from the second platform face 116b though it is contemplated that the gripping element 118 may extend in any direction from any portion, face, or surface of the restraint platform 102. Although the gripping element 118 is in the form of an arcuate handle in FIG. 1, the gripping element 118 may have any desired shape/form that is graspable by a medical professional.

As shown in FIGS. 1-3, the third platform portion 104c defines and laterally separates a first lateral platform side 120a of the restraint platform 102 from a second lateral platform side 120b of the restraint platform 102. The first lateral platform side 120a may include at least one of a first head restraint 122a, a first articulating assembly 124a, and a first sliding member 126a.

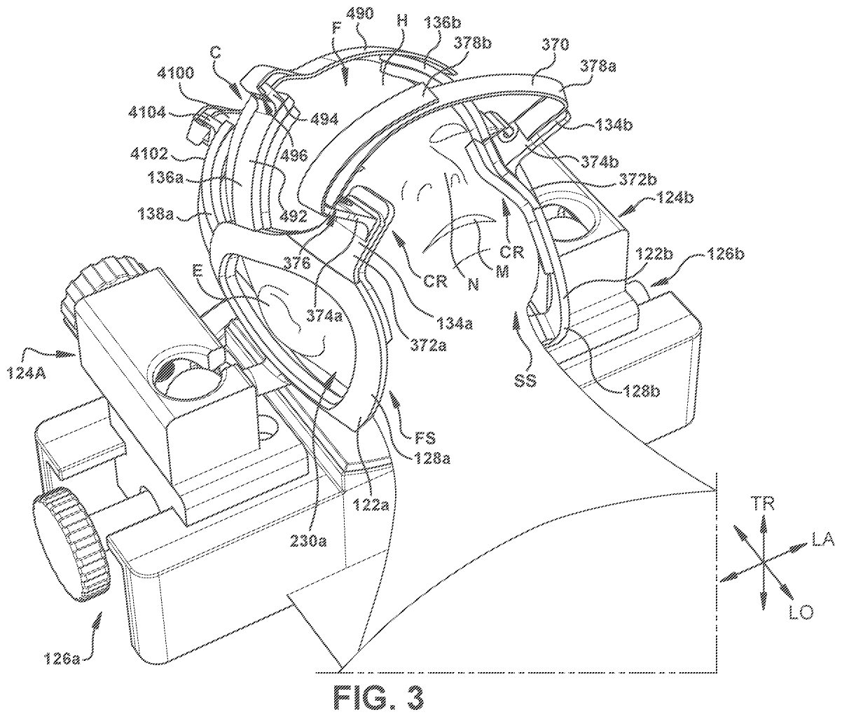

The first head restraint 122a is configured to selectively engage a first side FS of the subject's head. In particular, the first head restraint 122a has a first base portion 128a for engaging the first side FS of the subject's head H. The first base portion 128a may include a first earhole 230a defined by an inner periphery 232a of the first base portion 128a. The first earhole 230a is configured to be substantially aligned with at least a portion of an ear E of the subject S when the first head restraint 122a is engaged to the first side FS of the subject's head H. As shown in FIG. 3, the subject's ear E may extend at least partially through the first earhole 230a. It is also contemplated that the subject's ear E may merely be at least partially aligned with or adjacent to the first earhole 230a, or may be spaced apart from the first earhole 230a, for particular use environments.

As shown in FIGS. 1-4, the first base portion 128a may have at least one flange (shown here as flanges 134a, 136a, and 138a) extending therefrom for selectively engaging portions of the subject's head H on the first side FS of the subject's head H. For example, the first base portion 128a may have a first cheekbone flange 134a, a first forehead flange 136a, and a first crown flange 138a extending from an outer periphery 140a of the first base portion 128a. The first cheekbone flange 134a is configured to selectively engage a cheekbone region CR of the subject's head H, the first forehead flange 136a is configured to selectively engage a forehead F of the subject's head H, and the first crown flange 138a is configured to selectively engage a crown C of the subject's head H. The “crown” of the subject's head H, as defined herein, is a region of the subject's head H that is adjacent to or overlaps at least one of the subject's coronal suture, sagittal suture, parietal bone on the first side FS of the subject's head H, and parietal bone on a second side SS of the subject's head H.

At least one of the first base portion 128a, the first cheekbone flange 134a, the first forehead flange 136a, and the first crown flange 138a may be formed separately from and then subsequently connected to at least one other of the first base portion 128a, the first cheekbone flange 134a, the first forehead flange 136a, and the first crown flange 138a. Alternatively or additionally, at least one of the first base portion 128a, the first cheekbone flange 134a, the first forehead flange 136a, and the first crown flange 138a may be integrally formed with at least one other of the first base portion 128a, the first cheekbone flange 134a, the first forehead flange 136a, and the first crown flange 138a as a single-piece, unitary construct.

The first head restraint 122a may have a reusable or disposable first head cushion 142a interposed between the first side FS of the subject's head H and at least one of the first base portion 128a, the first cheekbone flange 134a, the first forehead flange 136a, and the first crown flange 138a. The first head cushion 142a provides a softer surface for the first side FS of the subject's head H to be engaged to than the non-cushioned first base portion 128a, first cheekbone flange 134a, first forehead flange 136a, and/or first crown flange 138a. In the example configuration shown in FIGS. 1-3, the first head cushion 142a is a single cushion that is provided on each of the first base portion 128a, the first cheekbone flange 134a, the first forehead flange 136a, and the first crown flange 138a. Alternatively, the first head cushion 142a may comprise a plurality of separate cushions. In such a configuration, at least one of the first base portion 128a, the first cheekbone flange 134a, the first forehead flange 136a, and the first crown flange 138a may have a cushion that is separate from a cushion that is on at least one other of the first base portion 128a, the first cheekbone flange 134a, the first forehead flange 136a, and the first crown flange 138a. The first head cushion 142a may be substantially permanently attached to the first head restraint 122a or may be configured to be selectively removed from the first head restraint 122a as desired.

The first articulating assembly 124a may be in the form of a first ball-and-socket assembly 124a. The first ball-and-socket assembly 124a may include a first ball portion 144a attached to a first ball stem 146a. The first ball stem 146a extends along a first stem axis 148a from the first ball portion 144a to the first base portion 128a to connect the first ball portion 144a to the first head restraint 122a. At least one of the first ball portion 144a, the first ball stem 146a, and the first base portion 128a may be formed separately from and then subsequently connected to at least one other of the first ball portion 144a, the first ball stem 146a, and the first base portion 128a. Alternatively or additionally, at least one of the first ball portion 144a, the first ball stem 146a, and the first base portion 128a may be integrally formed with at least one other of the first ball portion 144a, the first ball stem 146a, and the first base portion 128a as a single-piece, unitary construct.

The first ball-and-socket assembly 124a also includes a first socket portion 150a on, such as by being recessed into, a first ball-and-socket base 152a. Although first ball-and-socket assembly 124a is shown and described as having the first ball portion 144a attached to the first head restraint 122a and the first socket portion 150a on the first ball-and-socket base 152a, the first ball-and-socket assembly 124a may be configured such that the first ball portion 144a is attached to the first ball-and-socket base 152a and the first socket portion 150a is on the first head restraint 122a.

The first ball portion 144a is selectively received in the first socket portion 150a and is selectively articulatable relative to the first socket portion 150a. This arrangement provides the first head restraint 122a with at least three degrees of freedom of movement for the first head restraint 122a to conform to the first side FS of the subject's head H. For example, the first head restraint 122a may be pivoted at least in the longitudinal direction about a first center point 154a of the first ball portion 144a from the orientation shown in FIG. 1, the transverse direction about the first center point 154a of the first ball portion 144a from the orientation shown in FIG. 1, and the transverse or longitudinal direction about the first stem axis 148a from the orientation shown in FIG. 1.

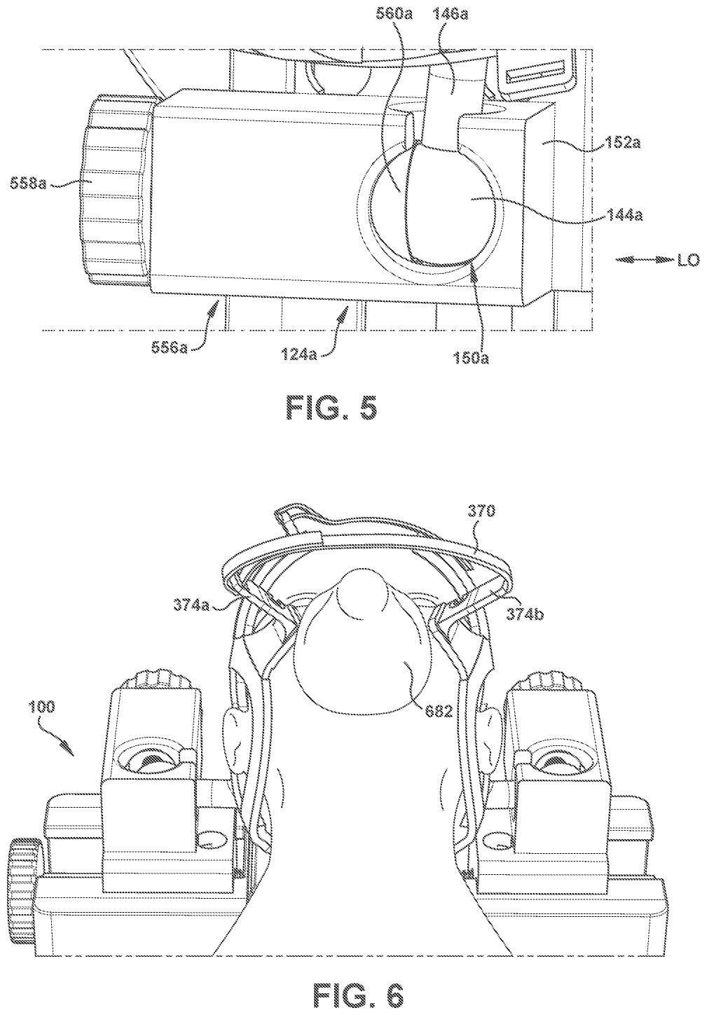

As shown in FIG. 5, the first ball-and-socket assembly 124a may include a first ball inhibiting mechanism 556a for selectively retaining the first head restraint 122a in a fixed position. The first ball inhibiting mechanism 556a may include a first inhibiting control knob 558a and a first ball stop 560a. When desired, the first inhibiting control knob 558a may be actuated, such as by being rotated in a first direction, to responsively cause the first ball stop 560a to move in the longitudinal direction toward the first ball portion 144a. The first ball stop 560a may be moved longitudinally until the first ball stop 560a engages the first ball portion 144a. Movement of the first ball portion 144a (and the first ball stem 146a connected thereto) relative to the first ball-and-socket base 152a is substantially inhibited once the first ball stop 560a engages the first ball portion 144a. Because the first head restraint 122a is connected to the first ball portion 144a via the first ball stem 146a, inhibiting the movement of the first ball portion 144a causes the first head restraint 122a to be retained in a fixed position. Therefore, the first head restraint 122a may be moved to a desired position in which the first head restraint 122a conforms to the first side FS of the subject's head H, and then locked in place by the first ball inhibiting mechanism 556a once the desired position is reached. When movement of the first head restraint 122a is once again desired, the first ball stop 560a may be disengaged from the first ball portion 144a by actuating the first inhibiting control knob 558a, such as by rotating the first inhibiting control knob 558a in a second direction opposite the first direction, to move the first ball stop 560a longitudinally away from the first ball portion 144a.

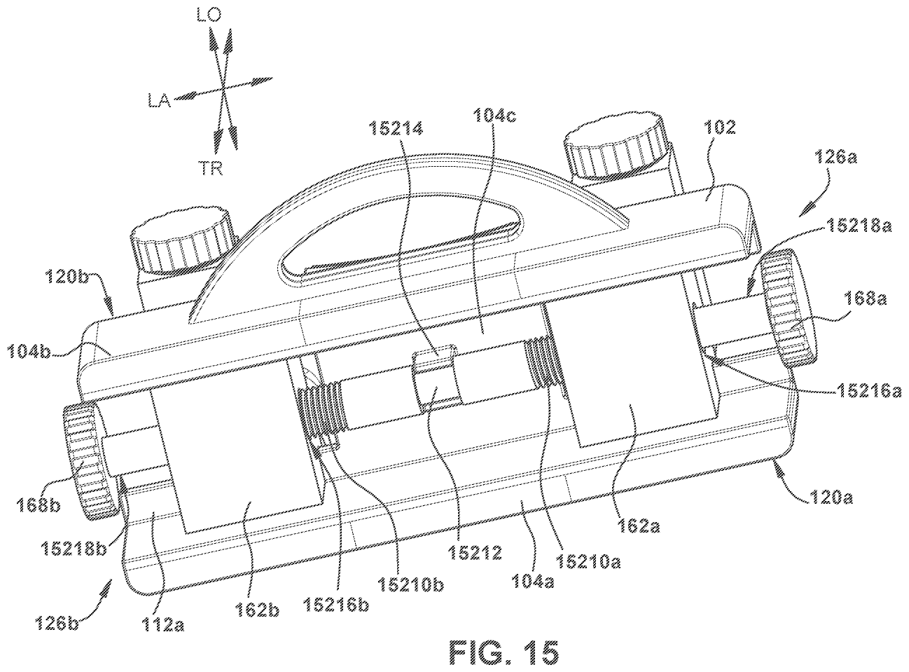

As shown in FIGS. 1-3, the first sliding mechanism 126a includes a first sliding base 162a. The first sliding base 162a includes first and second sliding base notches 264a, 266a that extend longitudinally inward from an exterior of the first sliding base 162a. The first sliding base notch 264a is configured to receive the first flange 112a, while the second sliding base notch 266a is configured to receive the second flange 112b. As shown in FIG. 15, the first sliding mechanism 126a also includes a first threaded portion 15210a of a drive shaft 15212. The drive shaft 15212 is rotatably mounted to the restraint platform 102 and substantially prevented from undesirably moving laterally and/or longitudinal by a stop clamp 15214 that extends transversely from the third platform portion 104c. The drive shaft 15212 extends laterally through a first threaded opening 15216a in the first sliding base 162a such that the first threaded portion 15210a is threadably engaged in the first threaded opening 15216a. A first shaft end 15218a of the drive shaft 15212 includes a first sliding control knob 168a. When desired, the first sliding control knob 168a may be actuated, such as by being rotated, to responsively cause the drive shaft 15212 to rotate relative to the first sliding base 162a. The rotating drive shaft 15212 responsively causes the first sliding base 162a to both move laterally along the first threaded portion 15210a and to concurrently slide laterally along the first and second flanges 112a, 112b of the restraint platform 102. Therefore, through actuation of the first sliding control knob 168a, the first sliding base 162a may be selectively moved laterally toward and away from the second lateral platform side 120b of the restraint platform 102.

The first sliding base 162a may be fixed to the first ball-and-socket base 152a and operably connected to the first head restraint 122a through the first ball-and-socket assembly 124a. The first head restraint 122a may thus be connected to the first lateral platform side 120a of the restraint platform 102 through the first ball-and-socket assembly 124a and the first sliding mechanism 126a. Selective lateral movement of the first sliding base 162a responsively causes the first head restraint 122a to move laterally relative to the restraint platform 102. Accordingly, the first sliding control knob 558a may be selectively actuated to cause the first head restraint 122a to move laterally toward a subject's head H received on the third platform portion 104c to engage the first side FS of the subject's head H, and selectively actuated to cause the first head restraint 122a to move laterally away the subject's head H to disengage the first side FS of the subject's head H.

Although the first articulating assembly 124a is shown and described as being a ball-and-socket assembly, the first articulating assembly 124a may be any other mechanism having a first portion connected to the first head restraint 122a, a second portion connected to the restraint platform 102 (such as via the first sliding mechanism 126a), and the first portion being selectively articulatable relative to the second portion to provide the first head restraint 122a with at least three degrees of freedom of movement.

The second lateral platform side 120b of the restraint platform 102 includes at least one of a second head restraint 122b, a second articulating assembly 124b, and a second sliding member 126b.

The second head restraint 122b is configured to selectively engage a second side SS of the subject's head H, lateral the first side FS of the subject's head H. In particular, the second head restraint 122b has a second base portion 128b for engaging the second side SS of the subject's head H. The second base portion 128b may include a second earhole 230b defined by an inner periphery 232b of the second base portion 128b. The second earhole 230b is configured to be substantially aligned with at least a portion of an ear E of the subject S when the second head restraint 122b is engaged to the second side SS of the subject's head H. The subject's ear E may extend at least partially through the second earhole 230b. It is also contemplated that the subject's ear E may merely be at least partially aligned with or adjacent to the second earhole 230b, or may be spaced apart from the second earhole 230b, for particular use environments.

As shown in FIGS. 1-4, the second base portion 128b may have at least one flange (shown here as flanges 134b, 136b, and 138b) extending therefrom for selectively engaging portions of the subject's head H on the second side SS of the subject's head H. For example, the second base portion 128b may have a second cheekbone flange 134b, a second forehead flange 136b, and a second crown flange 138b extending from an outer periphery 140b of the second base portion 128b. The second cheekbone flange 134b is configured to selectively engage a cheekbone region CR of the subject's head H, the second forehead flange 136b is configured to selectively engage the subject's forehead F, and the second crown flange 138b is configured to selectively engage the subject's crown C.

At least one of the second base portion 128b, the second cheekbone flange 134b, the second forehead flange 136b, and the second crown flange 138b may be formed separately from and then subsequently connected to at least one other of the second base portion 128b, the second cheekbone flange 134b, the second forehead flange 136b, and the second crown flange 138b. Alternatively or additionally, at least one of the second base portion 128b, the second cheekbone flange 134b, the second forehead flange 136b, and the second crown flange 138b may be integrally formed with at least one other of the second base portion 128b, the second cheekbone flange 134b, the second forehead flange 136b, and the second crown flange 138b as a single-piece, unitary construct.

The second head restraint 122b may have a reusable or disposable second head cushion 142b interposed between the second side SS of the subject's head H and at least one of the second base portion 128b, the second cheekbone flange 134b, the second forehead flange 136b, and the second crown flange 138b. The second head cushion 142b provides a softer surface for the second side SS of the subject's head H to be engaged to than the non-cushioned second base portion 128b, second cheekbone flange 134b, second forehead flange 136b, and/or second crown flange 138b. In the example configuration shown in FIGS. 1-3, the second head cushion 142b is a single cushion that is provided on each of the second base portion 128b, the second cheekbone flange 134b, the second forehead flange 136b, and the second crown flange 138b. Alternatively, the second restraint cushion 142b may comprise a plurality of separate cushions. In such a configuration, at least one of the second base portion 128b, the second cheekbone flange 134b, the second forehead flange 136b, and the second crown flange 138b may have a cushion that is separate from a cushion that is on at least one other of the second base portion 128b, the second cheekbone flange 134b, the second forehead flange 136b, and the second crown flange 138b. The second head cushion 142b may be substantially permanently attached to the second head restraint 122b or may be configured to be selectively removed from the second head restraint 122b as desired.

The second articulating assembly 124b may be in the form of a second ball-and-socket assembly 124b. The second ball-and-socket assembly 124b may include a second ball portion 144b attached to a second ball stem 146b. The second ball stem 146b extends along a second stem axis 148b from the second ball portion 144b to the second base portion 128b to connect the second ball portion 144b to the second head restraint 122b. At least one of the second ball portion 144b, the second ball stem 146b, and the second base portion 128b may be formed separately from and then subsequently connected to at least one other of the second ball portion 144b, the second ball stem 146b, and the second base portion 128b. Alternatively or additionally, at least one of the second ball portion 144b, the second ball stem 146b, and the second base portion 128b may be integrally formed with at least one other of the second ball portion 144b, the second ball stem 146b, and the second base portion 128b as a single-piece, unitary construct.

The second ball-and-socket assembly 124b also includes a second socket portion 150b on, such as by being recessed into, a second ball-and-socket base 152b. Although second ball-and-socket assembly 124b is shown and described as having the second ball portion 144b attached to the second head restraint 122b and the second socket portion 150b on the second ball-and-socket base 152b, the first ball-and-socket assembly 124b may be configured such that the second ball portion 144b is attached to the second ball-and-socket base 152b and the second socket portion 150b is on the second head restraint 122b.

The second ball portion 144b is selectively received in the second socket portion 150b and is selectively articulatable relative to the second socket portion 150b. This arrangement provides the second head restraint 122b with at least three degrees of freedom of movement for the second head restraint 122b to conform to the second side SS of the subject's head H. For example, the second head restraint 122b may be pivoted at least in the longitudinal direction about a second center point 154b of the second ball portion 144b from the orientation shown in FIG. 1, the transverse direction about the second center point 154b of the second ball portion 144b from the orientation shown in FIG. 1, and the transverse or longitudinal direction about the second stem axis 148b from the orientation shown in FIG. 1.

The second ball-and-socket assembly 124b may include a second ball inhibiting mechanism 556b for selectively retaining the second head restraint 122b in a fixed position. The second ball inhibiting mechanism 556b may mirror the first ball inhibiting mechanism 556a shown in detail in FIG. 5. The second ball inhibiting mechanism 556b may include a second inhibiting control knob 558b and a second ball stop 560b. When desired, the second inhibiting control knob 558b may be actuated, such as by being rotated in a first direction, to responsively cause the second ball stop 560b to move in the longitudinal direction toward the second ball portion 144b. The second ball stop 560b may be moved longitudinally until the second ball stop 560b engages the second ball portion 144b. Movement of the second ball portion 144b (and the second ball stem 146b connected thereto) relative to the second ball-and-socket base 152b is substantially inhibited once the second ball stop 560b engages the second ball portion 144b. Because the second head restraint 122b is connected to the second ball portion 144b via the second ball stem 146b, inhibiting the movement of the second ball portion 144b causes the second head restraint 122b to be retained in a fixed position. Therefore, the second head restraint 122b may be moved to a desired position in which the second head restraint 122b conforms to the second side SS of the subject's head H, and then locked in place by the second ball inhibiting mechanism 556b once the desired position is reached. When movement of the second head restraint 122b is once again desired, the second ball stop 560b may be disengaged from the second ball portion 144b by actuating the second inhibiting control knob 558b, such as by rotating the second inhibiting control knob 558b in a second direction opposite the first direction, to move the second ball stop 560b longitudinally away from the second ball portion 144b.

As shown in FIGS. 1-3, the second sliding mechanism 126b includes a second sliding base 162b. The second sliding base 162b includes first and second sliding base notches 264b, 266b that extend longitudinally inward from an exterior of the second sliding base 162b. The first sliding base notch 264b is configured to receive the first flange 112a, while the second sliding base notch 266b is configured to receive the second flange 112b. As shown in FIG. 15, the second sliding mechanism 126b also includes a second threaded portion 15210b of the drive shaft 15212. The drive shaft 15212 extends laterally through a second threaded opening 15216b in the second sliding base 162b such that the second threaded portion 15210b is threadably engaged in the second threaded opening 15216b. A second shaft end 15218b of the drive shaft 15212 includes a second sliding control knob 168b. When desired, the second sliding control knob 168b may be actuated, such as by being rotated, to responsively cause the drive shaft 15212 to rotate relative to the second sliding base 162b. The rotating drive shaft 15212 responsively causes the second sliding base 162b to both move laterally along the second threaded portion 15210b and to concurrently slide laterally along the first and second flanges 112a, 112b of the restraint platform 102. Therefore, through actuation of the second sliding control knob 168b, the second sliding base 162b may be selectively moved laterally toward and away from the first lateral platform side 120a of the restraint platform 102.

Because a single drive shaft 15212 is threadably connected to both the first and second sliding bases 162a, 162b, selective actuation of either of the first and second control knobs 168a, 168b responsively causes both of the first and second sliding bases 162a, 162b to simultaneously be moved laterally toward or away one another. It is contemplated, however, that the first and second sliding mechanisms 126a, 126b may include separate drive shafts 15212 so that selective rotation of one of the first and second control knobs 168a, 168b causes only the associated one of the first and second sliding bases 162a, 162b to move laterally.

The second sliding base 162b may be fixed to the second ball-and-socket base 152b and operably connected to the second head restraint 122b through the second ball-and-socket assembly 124b. The second head restraint 122b may thus be connected to the second lateral platform side 120b of the restraint platform 102 through the second ball-and-socket assembly 124b and the second sliding mechanism 126b. Selective lateral movement of the second sliding base 162b responsively causes the second head restraint 122b to move laterally relative to the restraint platform 102. Accordingly, the second sliding control knob 558b may be selectively actuated to cause the second head restraint 122b to move laterally toward a subject's head H received on the third platform portion 104c to engage the second side SS of the subject's head H, and selectively actuated to cause the second head restraint 122b to move laterally away the subject's head H to disengage the second side SS of the subject's head H.

Although the second articulating assembly 124b is shown and described as being a ball-and-socket assembly, the second articulating assembly 124b may be any other mechanism having a first portion connected to the second head restraint 122b, a second portion connected to the restraint platform 102 (such as via the second sliding mechanism 126b), and the first portion being selectively articulatable relative to the second portion to provide the second head restraint 122b with at least three degrees of freedom of movement.

Via the first and second sliding mechanisms 126a, 126b, the first and second head restraints 122a, 122b may also be selectively movable in the lateral direction relative to the restraint platform 102. Furthermore, the first and second ball-and-socket assemblies 124a, 124b permit the first and second head restraints 122a, 122b to articulate relative to one another. Therefore, the first and second head restraints 122a, 122b may be adjustable relative to the restraint platform 102 and the subject's head H when received thereon and at least partially independently adjusted relative to one another in order to conform to their respective sides FS, SS of the subject's head H.

As shown in FIGS. 3-4 and 6-7, the head restraint assembly 100 may include at least one strap 370, 490, 4100 that extends from one of the first and second head restraints 122a, 122b to the other of the first and second head restraint 122a, 122b. The at least one strap may be configured for selectively tightening the first and second head restraints 122a, 122b against the subject's head H to help retain the first and second head restraints 122a, 122b engaged to the first and second sides FS, SS of the subjects head H, as well as help prevent the subject's head H from undesirably egressing from the head restraint assembly 100. Tightening the first and second head restraints 122a, 122b against the subject's head H with the at least one strap may also help substantially immobilize the subject's head H when desired.

For example, the head restraint assembly 100 may include a cheekbone strap 370 that extends between the first and second cheekbone flanges 134a, 136b. In particular, the first cheekbone flange 134a may have a first cheekbone connecting portion 372a extending from the outer periphery 140a of the first base portion 128a, and a first cheekbone attachment portion 374a extending substantially perpendicularly from the first cheekbone connecting portion 372a. The first cheekbone attachment portion 374a may include a cheekbone aperture 376 extending therethrough that is configured to have at least a portion of the cheekbone strap 370 passed therethrough. The second cheekbone flange 136b may have a second cheekbone connecting portion 372b extending from the outer periphery 140b of the second base portion 128b, and a second cheekbone attachment portion 374b extending substantially perpendicularly from the second cheekbone connecting portion 372b. A first end 378a of cheekbone strap 370 may be fixedly connected to at least the second cheekbone attachment portion 374b. A second end 378b of the cheekbone strap 370 may be selectively looped through the cheekbone aperture 376 of the first cheekbone attachment portion 374a and pulled toward the second cheekbone attachment portion 374b to selectively tighten the first and second cheekbone flanges 134a, 136b against the subject's cheekbone regions CR. Once the first and second cheekbone flanges 134a, 136b are tightened as desired, the second end 378b of the cheekbone strap 370 may be selectively attached to another portion of the cheekbone strap 370 to retain the desired tightness of the first and second cheekbone flanges 134a, 136b. The second end 378b of the cheekbone strap 370 may be attached to the other portion of the cheekbone strap 370 via at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

Although the head restraint assembly 100 is shown and described as having the cheekbone aperture 370 extending through the first cheekbone attachment portion 374 and the first end 378a of the cheekbone strap 370 being fixedly connected to the second cheekbone attachment portion 374b, the head restraint assembly 100 may be configured such that the cheekbone aperture 376 extends through the second cheekbone attachment portion 374b and the first end 378a of the cheekbone strap 370 is fixedly connected to the first cheekbone attachment portion 374a. Further, although the first end 378a of the cheekbone strap 370 is described as being fixedly connected to the second cheekbone attachment portion 374b, the first end 378a of the cheekbone strap 370 may be selectively fixedly connected to the to the second cheekbone attachment portion 374b and removable therefrom so that the cheekbone strap 370 may be replaced as desired, with a similar, replacement cheekbone strap 370 or with a cheekbone strap including at least one different physical property (e.g., length, thickness, width, material, flexibility) than the original cheekbone strap 370.

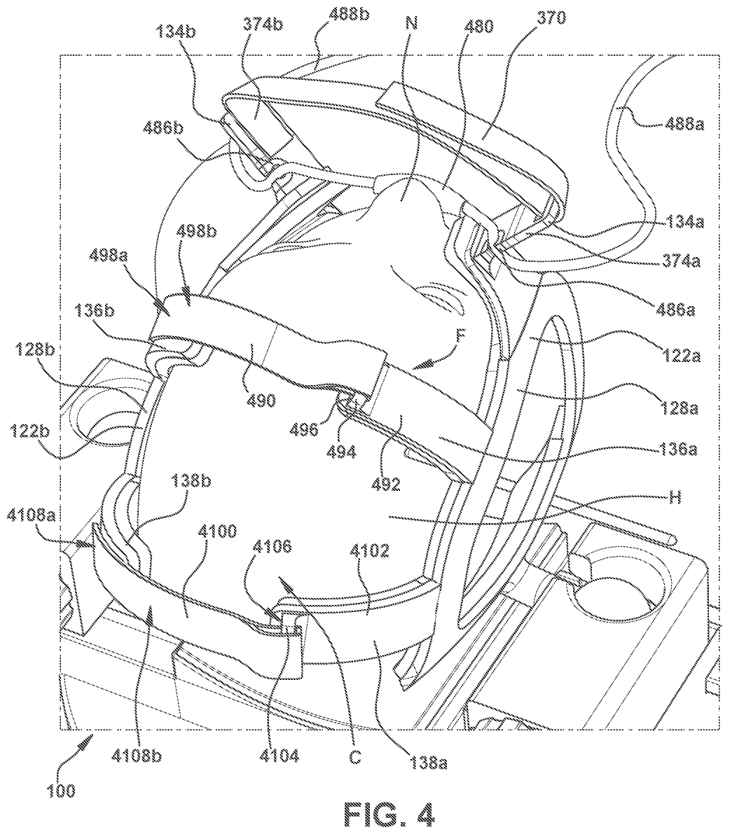

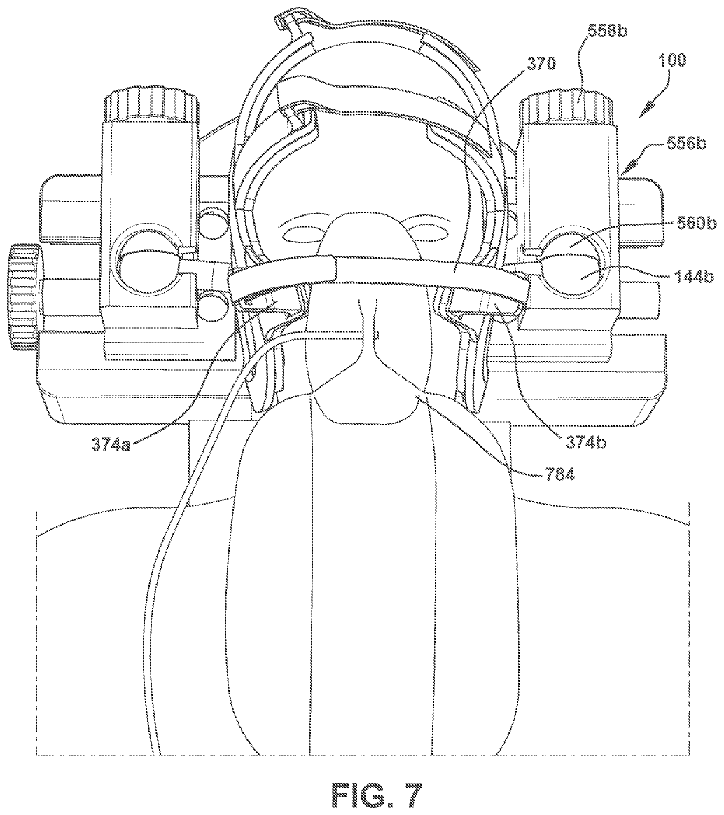

As shown in FIGS. 3-4 and 6-7, the first and second cheekbone attachment portions 374a, 374b are configured so that the cheekbone strap 370 is spaced from the subject's head H when in use. This spacing permits the medical professional to access the subject's nose N and mouth M as desired. For example, the space between the cheekbone strap 370 and the subject's head H permits the medical professional to attach/use at least one of a nasal cannula 480 (FIG. 4), a gas (such as, for example, oxygen) delivery mask 682 (FIG. 6), a non-rebreather mask 784 (FIG. 7), any other device or combination thereof which accesses the subject's nose N and/or mouth M when the subject's head H is restrained by the head restraint assembly 100.

As shown in FIG. 4, the first cheekbone attachment portion 374a may include a first cheekbone notch 486a that extends inward from an exterior of the first cheekbone attachment portion 374a. The second cheekbone attachment portion 374b may include a second cheekbone notch 486b that extends inward from an exterior of the second cheekbone attachment portion 374b. The first and second cheekbone notches 486a, 486b may each be configured to selectively receive and selectively retain at least a portion of a cross-sectional perimeter of a medical tubing 488a, 488b therein. Although the first and second cheekbone attachment portions 374a, 374b are shown as having one cheekbone notch 486a, 486b each, at least one of the first and second cheekbone attachment portions 374a, 374b may have no cheekbone notches, one cheekbone notch, or a plurality of cheekbone notches. Further, instead of having a cheekbone notch 486a, 486b, at least one of the first and second cheekbone attachment portions 374a, 374b may include other features that are configured to selectively receive and selectively retain a medical tubing 488a, 488b. For example, at least one of the first and second cheekbone attachment portions 374a, 374b may include a clip attached thereto that is configured to selectively receive and selectively retain a medical tubing 488a, 488b therein.

As shown in FIG. 4, the head restraint assembly 100 may include a forehead strap 490 that extends between the first and second forehead flanges 136a, 138b. In particular, the first forehead flange 136a may have a forehead connecting portion 492 extending from the outer periphery 140a of the first base portion 128a, and a forehead attachment portion 494 extending substantially perpendicularly from the forehead connecting portion 492. The forehead attachment portion 494 may include a forehead aperture 496 extending therethrough that is configured to have at least a portion of the forehead strap 490 inserted therethrough. A first end 498a of forehead strap 490 may be fixedly connected to at least the second forehead flange 138b. A second end 498b of the forehead strap 490 may be selectively looped through the forehead aperture 496 of the forehead attachment portion 494 and pulled toward the second forehead flange 138b to selectively tighten the first and second forehead flanges 136a, 138b against the subject's forehead F. Once the first and second forehead flanges 136a, 138b are tightened as desired, the second end 498b of the forehead strap 490 may be selectively attached to another portion of the forehead strap 490 to retain the desired tightness of the first and second forehead flanges 136a, 138b. The second end 498b of the forehead strap 490 may be attached to the other portion of the forehead strap 490 via at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

Although the head restraint assembly 100 is shown and described as having the forehead aperture 496 extending through a portion of the first forehead flange 136a and the first end 498a of the forehead strap 490 being fixedly connected to the second forehead flange 136b, the head restraint assembly 100 may be configured such that the forehead aperture 496 extends through a portion of the second forehead flange 136b and the first end 498a of the forehead strap 490 is fixedly connected to the first forehead flange 136a. Further, although the first end 498a of the forehead strap 490 is described as being fixedly connected to the second forehead flange 136b, the first end 498a of the forehead strap 490 may be selectively fixedly connected to the to the second forehead flange 136b and removable therefrom so that the forehead strap 490 may be replaced as desired, with a similar, replacement forehead strap 490 or with a forehead strap including at least one different physical property (e.g., length, thickness, width, material, flexibility) than the original forehead strap 490.

The head restraint assembly 100 may include a crown strap 4100 that extends between the first and second crown flanges 138a, 138b. In particular, the first crown flange 138a may have a crown connecting portion 4102 extending from the outer periphery 140a of the first base portion 128a, and a crown attachment portion 4104 extending substantially perpendicularly from the crown connecting portion 4102. The crown attachment portion 4104 may include a crown aperture 4106 extending therethrough that is configured to have at least a portion of the crown strap 4100 inserted therethrough. A first end 4108a of crown strap 4100 may be fixedly connected to at least the second crown flange 138b. A second end 4108b of the crown strap 4100 may be selectively looped through the crown aperture 4106 of the crown attachment portion 4104 and pulled toward the second crown flange 138b to selectively tighten the first and second crown flanges 138a, 138b against the subject's crown C. Once the first and second crown flanges 138a, 138b are tightened as desired, the second end 4108b of the crown strap 4100 may be selectively attached to another portion of the crown strap 4100 to retain the desired tightness of the first and second crown flanges 138a, 138b. The second end 4108b of the crown strap 4100 may be attached to the other portion of the crown strap 4100 via at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

Although the head restraint assembly 100 is shown and described as having the crown aperture 4106 extending through a portion of the first crown flange 138a and the first end 4108a of the crown strap 4100 being fixedly connected to the second crown flange 138b, the head restraint assembly 100 may be configured such that the crown aperture 4106 extends through a portion of the second crown flange 138b and the first end 4108a of the crown strap 4100 is fixedly connected to the first crown flange 138a. Further, although the first end 4108a of the crown strap 4100 is described as being fixedly connected to the second crown flange 138b, the first end 4108a of the crown strap 4100 may be selectively fixedly connected to the to the second crown flange 138b and removable therefrom so that the crown strap 4100 may be replaced as desired, with a similar, replacement crown strap 4100 or with a crown strap including at least one different physical property (e.g., length, thickness, width, material, flexibility) than the original crown strap 4100.

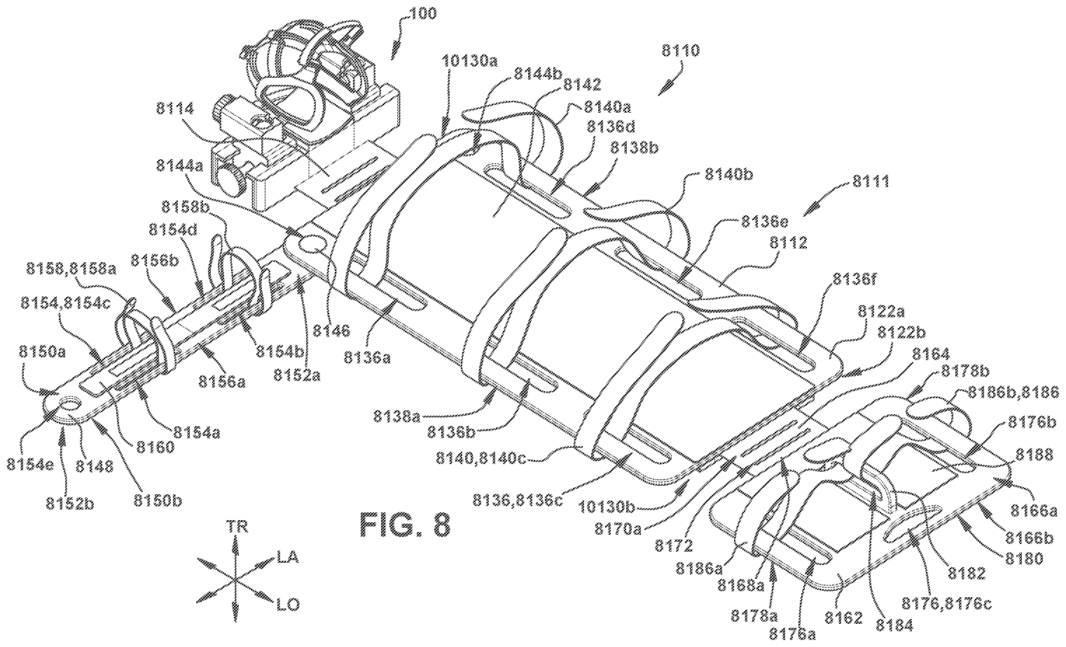

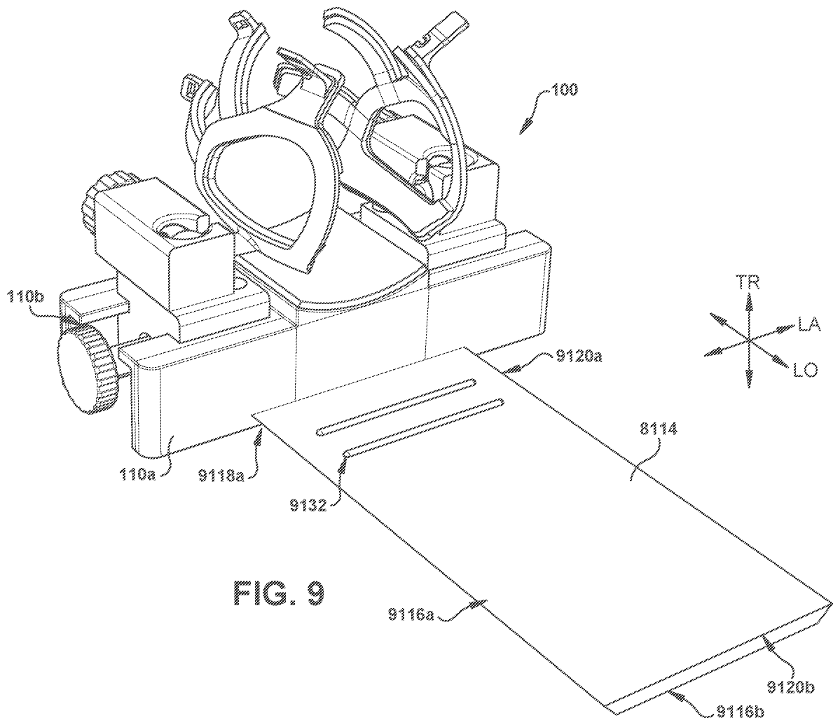

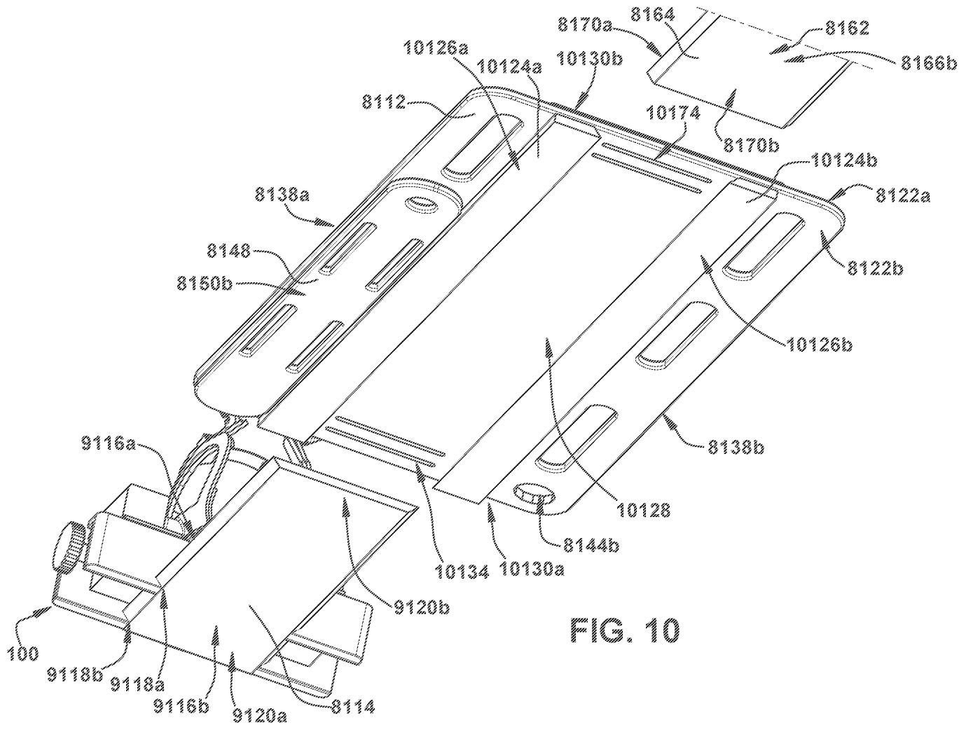

As shown in FIG. 8, the head restraint assembly 100 may be included as part of a restraint assembly 8110 that is for helping to restraint the subject S. The restraint assembly 8110 also includes a body stabilizer assembly 8111 having a body stabilizer 8112 that is configured to receive a portion of the subject's body B below the subject's head H. The head restraint assembly 100 may be removably attached to the body stabilizer 8112 by a head connector 8114. As shown in FIGS. 9-10, the head connector 8114 has transversely opposing first and second head connector surfaces 9116a, 9116b that are separated from one another in the transverse direction. The head connector 8114, in cross-section, may have a substantially triangle frustum shape. Therefore, the lateral width of the head connector 8114 may decrease from the first head connector surface 9116a to the second head connector surface 9116b. However, it is contemplated that the head connector 8114 may have any desired cross-sectional shape such as, but not limited to, rectangular, curved, and/or rectilinear.

The first and second columns 110a, 110b of the first and second platform portions 104a, 104b may include a first and second head connector notches 9118a, 9118b, respectively, extending laterally therethrough. The first and second head connector notches 9118a, 9118b are configured to match the cross-sectional shape of the head connector 8114 so that a first head connector end portion 9120a of the head connector 8114 may be selectively inserted longitudinally through the first and second connector notches 9118a, 9118b to join the head connector 8114 to the head restraint assembly 100. The first and second head connector notches 9118a, 9118b may be configured so that a press-fit and/or frictional engagement is created between the head restraint assembly 100 and the head connector 8114 when the head connector 8114 extends through the first and second head connector notches 9118a, 9118b. The press-fit and/or frictional engagement between the head restraint assembly 100 and the head connector 8114 may help prevent the head connector 8114 from being undesirably detached from the head restraint assembly 100.

Alternatively, or additionally to the press-fit and/or frictional engagement maintaining the head connector 8114 connected to the head restraint assembly 100, the head connector 8114 may be selectively or substantially permanently connected to at least one of the first platform portion 104a, the second platform portion 104b, and the third platform portion 104c by one or more fasteners. Each fastener that connects the head connector 8114 to the head restraint assembly 100 may be at least one of an adhesive (such as a permanent adhesive epoxy), tape (such as a high performance acrylic adhesive double-sided tape), a screw (such as a plastic or carbon fiber screw), a nail (such as a plastic or carbon fiber nail), a hook and loop fastener, a snap fastener, any suitable metallic or non-metallic fastener, and/or any other suitable fastener or combination of fasteners. It is also contemplated that the head connector 8114 may be integrally formed as a single-piece, unitary construct with at least one of the first platform portion 104a, the second platform portion 104b, and the third platform portion 104c.

As shown in FIGS. 8 and 10, the body stabilizer 8112 has transversely opposing first and second stabilizer surfaces 8122a, 8122b that are separated from one another in the transverse direction. First and second stabilizer bases 10124a, 10124b may extend transversely from the second stabilizer surface 8122b. The first and second stabilizer bases 10124a, 10124b may each have a free end surface 10126a, 10126b that faces away from the second stabilizer surface 8122b in the longitudinal direction. Both free end surfaces 10126a, 10126b may be coplanar in order to help stabilize the body stabilizer 8112 when in use. At least one of the body stabilizer 8112, the first stabilizer base 10124a, and the second stabilizer base 10124b may be formed separately from and then subsequently connected to at least one other of the body stabilizer 8112, the first stabilizer base 10124a, and the second stabilizer base 10124b. Alternatively or additionally, at least one of the body stabilizer 8112, the first stabilizer base 10124a, and the second stabilizer base 10124b may be integrally formed with at least one other of the body stabilizer 8112, the first stabilizer base 10124a, and the second stabilizer base 10124b as a single-piece, unitary construct.

The first and second stabilizer bases 10124a, 10124b may be separated from one another by a laterally extending stabilizer gap 10128. The first and second stabilizer bases stabilizer gap may shape the stabilizer gap 10128 to match the cross-sectional shape of the head connector 8114 so that a second head connector end portion 9120b of the head connector 8114 may be selectively inserted longitudinally into the stabilizer gap 10128 between the first and second stabilizer bases 10124a, 10124b to join the head connector 8114 to the body stabilizer 8112 on a first longitudinal stabilizer side 10130a of the body stabilizer 8112. The first stabilizer base 10124a, the second stabilizer base 10124b, and the stabilizer gap 10128 may be configured so that a press-fit and/or frictional engagement between the body stabilizer 8112 and the head connector 8114 is created when the head connector 8114 is inserted into the stabilizer gap 10128. The press-fit and/or frictional engagement between the body stabilizer 8112 and the head connector 8114 may help prevent the head connector 8114 from being undesirably detached from the body stabilizer 8112.

As shown in FIGS. 9-10, the first head connector surface 9116a may also include at least one head locking projection 9132 that is configured to engage at least one head locking detent 10134 in the second stabilizer surface 8122b when the head connector 8114 is joined to the body stabilizer 8112. The engagement between the head locking projection 9132 and the head locking detent 10134 may also help prevent the head connector 8114 from being undesirably detached from the body stabilizer 8112. It is also contemplated that the head connector 8114 may be integrally formed with the body stabilizer 8112 as a single-piece, unitary construct.

As shown in FIG. 8, the body stabilizer 8112 may include at least one stabilizer aperture 8136 (shown here as stabilizer apertures 8136a, 8136b, 8136c, 8136d, 8136e, and 8136f) extending transversely therethrough. In the example configuration of FIG. 8, the body stabilizer 8112 includes first, second, and third stabilizer apertures 8136a, 8136b, 8136c longitudinally positioned along a first lateral stabilizer side 8138a of the body stabilizer 8112. The body stabilizer 8112 also includes fourth, fifth, and sixth stabilizer apertures 8136d, 8136e, 8136f longitudinally positioned along a second lateral stabilizer side 8138b of the body stabilizer 8112. The first, second, and third stabilizer apertures 8136a, 8136b, 8136c may be longitudinally aligned or longitudinally offset from the fourth, fifth, and sixth stabilizer apertures 8136d, 8136e, 8136f. The stabilizer apertures 8136a, 8136b, 8136c, 8136d, 8136e, 8136f may be used by at least one medical professional to grip the body stabilizer 8112 and transport a restrained subject.

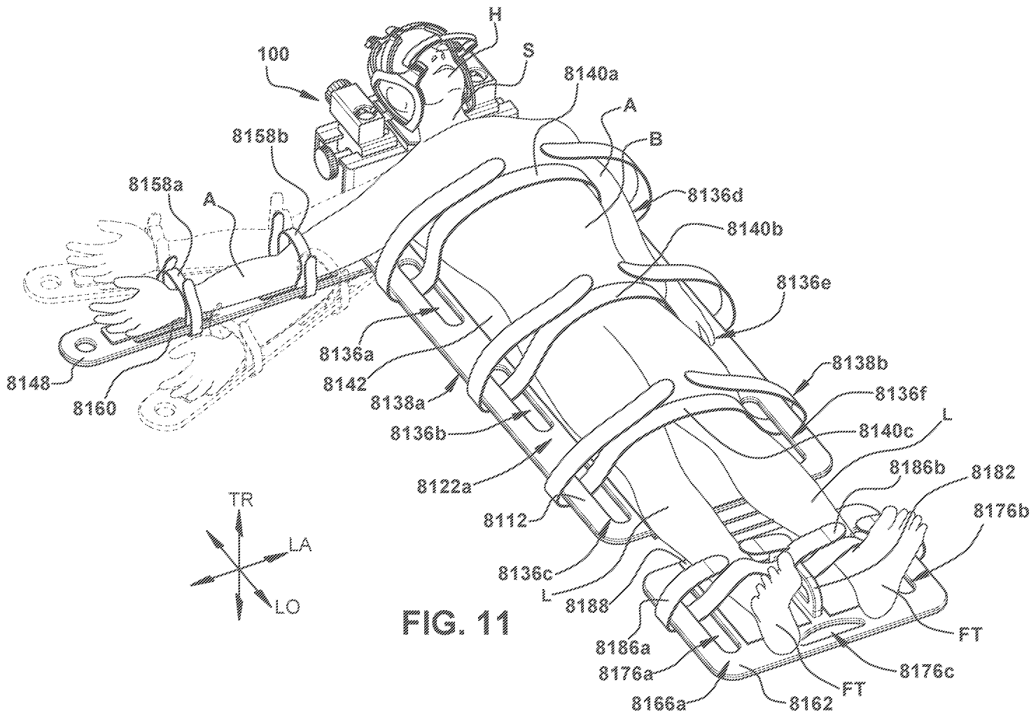

As shown in FIGS. 8 and 11, the body stabilizer assembly 8111 may include at least one restraining strap 8140 (shown here as restraining straps 8140a, 8140b, and 8140c) configured to be selectively attached to the body stabilizer 8112 and selectively prevent the egress of the subject S from the body stabilizer 8112. The at least one restraining strap 8140 may also be configured to immobilize at least a portion of the subject's body B on the body stabilizer 8112. The body stabilizer assembly 8111 may include, for example, first, second, and third restraining straps 8140a, 8140b, 8140c. The first restraining strap 8140a may be looped through and extend between the first and fourth stabilizer apertures 8136a, 8136d, the second restraining strap 8140b may be looped through and extend between the second and fifth stabilizer apertures 8136b, 8136e, and the third restraining strap 8140c may be looped through and extend between the third and sixth stabilizer apertures 8136c, 8136f.

Each of the first, second, and third restraining straps 8140a, 8140b, 8140c may be selectively longitudinally movable along their respective stabilizer apertures 8136a, 8136b, 8136c, 8136d, 8136e, 8136f. This longitudinal movement allows each of the first, second, and third restraining straps 8140a, 8140b, 8140c to be placed in selected longitudinal positions along the subject's body B in order to correspond to the subject's anatomy. Each of the first, second, and third restraining straps 8140a, 8140b, 8140c may be selectively retained attached to the body stabilizer 8112 and engaged to the subject S through the use of at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

The first stabilizer surface 8122a may have a reusable or disposable stabilizer cushion 8142 provided thereon that is configured to be interposed between the subject's body B and the first stabilizer surface 8122a. The stabilizer cushion 8142 provides a softer surface for the subject's body B to be laid on than the substantially rigid first stabilizer surface 8122a. The stabilizer cushion 8142 may be substantially permanently attached to the first stabilizer surface 8122a or may be configured to be selectively removed from the first stabilizer surface 8122a as desired.

As shown in FIGS. 8 and 10, at least one of the first and second lateral stabilizer sides 8138a, 8138b may include a winglet attachment aperture 8144a, 8144b extending transversely therethrough. For example, the body stabilizer 8112 shown in FIGS. 8 and 10 includes a first winglet attachment aperture 8144a on the first lateral stabilizer side 8138a and a second winglet attachment aperture 8144b on the second lateral stabilizer side 8138b. Each of the first and second winglet attachment apertures 8144a, 8144b is configured to selectively receive a winglet attachment projection 8146 of a winglet 8148 to attach the winglet 8148 to the body stabilizer 8112.

The winglet 8148 is configured to selectively receive an arm A of the subject S and has transversely opposing first and second winglet surfaces 8150a, 8150b that are separated from one another in the transverse direction. The winglet attachment projection 8146 extends transversely from the first winglet surface 8150a on a first lateral winglet side 8152a of the winglet 8148. When the winglet 8148 is attached to the body stabilizer 8112, the first winglet surface 8150a abuts or is adjacent to the second stabilizer surface 8122b. As shown in FIG. 10, the second winglet surface 8150b of the attached winglet 8148 may be substantially coplanar with the free end surfaces 10126a, 10126b of the first and second stabilizer bases 10124a, 10124b so as to not significantly disrupt the stability of the body stabilizer 8112.

The winglet attachment projection 8146 and the first and second winglet attachment apertures 8144a, 8144b are configured such that the winglet 8148 may be selectively pivoted relative to the body stabilizer 8112 to a desired angular position for receiving the subject's arm A. Pivoting the winglet 8148 to a desired angular position may include disconnecting the winglet 8148 from the body stabilizer 8112 by removing the winglet attachment projection 8146 from one of the first and second winglet attachment apertures 8144a, 8144b, pivoting the winglet 8148 relative to the body stabilizer 8112 to the desired angular position, and then reattaching the winglet 8148 to the body stabilizer with the winglet 8148 in the desired angular position. The winglet attachment projection 8146 and the first and second winglet attachment apertures 8144a, 8144b may be keyed or indexed such that the winglet 8148 is pivotable at predetermined angular increments relative to the body stabilizer 8112 (as shown in the configuration of FIGS. 8, 10, and 11), or the winglet 8148 may be freely pivotable relative to the body stabilizer 8112. The keying or indexing may comprise substantially shaping at least a portion of the winglet attachment projection 8146′ and the first and second winglet attachment apertures 8144a′, 8144b′ as an octagon, any other polygon, or any other desired shape.

As shown in FIGS. 8 and 10, the winglet 8148 may include at least one winglet aperture 8154 (shown here as winglet apertures 8154a, 8154b, 8154c, 8154d, and 8154e) extending transversely therethrough. In the example configuration and orientation of FIG. 8, the winglet 8148 includes first and second winglet apertures 8154a, 8154b laterally positioned along a first longitudinal winglet side 8156a of the winglet 8148. The winglet 8148 also includes third and fourth winglet apertures 8154c, 8154d laterally positioned along a second longitudinal winglet side 8156b of the winglet 8148. The first and second winglet apertures 8154a, 8154b may be laterally aligned or laterally offset from the third and fourth winglet apertures 8154c, 8154d. A fifth winglet aperture 8154e may extend transversely through the winglet 8148 on a second lateral winglet side 8152b of the winglet 8148. The winglet apertures 8154a, 8154b, 8154c, 8154d, 8154e may be used by at least one medical professional to grip the winglet 8148 when transporting a restrained subject S and/or to manipulate the subject's arm A.

As shown in FIGS. 8 and 11, the restraint assembly 8110 may include at least one winglet strap 8158 (shown here as winglet straps 8158a and 8158b) configured to be selectively attached to the winglet 8148 and selectively prevent the egress of the subject's arm A from the winglet 8148. The at least one winglet strap 8158 may also be configured to immobilize at least a portion of the subject's arm A on the winglet 8148. The restraint assembly 8110 may include, for example, first and second winglet straps 8158a, 8158b. The first winglet strap 8158a may be looped through and extend between the first and third winglet apertures 8154a, 8154c, and the second winglet strap 8158b may be looped through and extend between the second and fourth winglet apertures 8154b, 8154d.

Each of the first and second winglet straps 8158a, 8158b may be selectively laterally movable along their respective winglet apertures 8154a, 8154b, 8154c, 8154d. This lateral movement allows each of the first and second winglet straps 8158a, 8158b to be placed in selected lateral positions along the subject's arm A in order to correspond to the subject's anatomy. Each of the first and second winglet straps 8158a, 8158b may be selectively retained attached to the winglet 8148 and engaged to the subject's arm A through the use of at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

The first winglet surface 8150a may have a reusable or disposable winglet cushion 8160 provided thereon that is configured to be interposed between the subject's arm A and the first winglet surface 8150a. The winglet cushion 8160 provides a softer surface for the subject's arm A to be laid on than the substantially rigid first winglet surface 8150a. The winglet cushion 8160 may be substantially permanently attached to the first winglet surface 8150a or may be configured to be selectively removed from the first winglet surface 8150a as desired.

Although the body stabilizer assembly 8111 is shown as having only one winglet 8148, the body stabilizer assembly 8111 may have any number of winglets 8148. For example, one winglet 8148 may be joined to the first winglet attachment aperture 8144a, while a second winglet 8148 may be attached to the second winglet attachment aperture 8144b.

As shown in FIGS. 8 and 10-11, the body stabilizer assembly 8111 may include a foot platform 8162 that is configured to receive a portion of the subject's feet FT and/or a portion of the subject's legs L. The foot platform 8162 may be removably attached to the body stabilizer 8112 by a foot connector 8164. The foot platform 8162 includes transversely opposing first and second foot surfaces 8166a, 8166b that are separated from one another in the transverse direction. The first foot surface 8166a is configured to receive the subject's feet FT and/or legs L. The second foot surface 8166b may be selectively or substantially permanently connected to a first foot connector end portion 8168a by one or more fasteners. Each fastener that connects the foot connector 8164 to the foot platform 8162 may be at least one of an adhesive (such as a permanent adhesive epoxy), tape (such as a high performance acrylic adhesive double-sided tape), a screw (such as a plastic or carbon fiber screw), a nail (such as a plastic or carbon fiber nail), a hook and loop fastener, a snap fastener, any suitable metallic or non-metallic fastener, and/or any other suitable fastener or combination of fasteners. Alternatively, the foot platform 8162 and the foot connector 8164 may be integrally formed together as a single-piece, unitary construct.

The foot connector 8164 has transversely opposing first and second foot connector surfaces 8170a, 8170b that are separated from one another in the transverse direction. The foot connector 8164, in cross-section, may have a substantially triangle frustum shape similar to the head connector 8114. Therefore, the lateral width of the foot connector 8164 may decrease from the first foot connector surface 8170a to the second foot connector surface 8170b. However, it is contemplated that the foot connector 8164 may have any desired cross-sectional shape such as, but not limited to, rectangular, curved, and/or rectilinear.

The first and second stabilizer bases 10124a, 10124b may shape the stabilizer gap 10128 between the first and second stabilizer bases 10124a, 10124b to match the cross-sectional shape of the foot connector 8164 so that a second foot connector end portion 8168b of the foot connector 8164 may be selectively inserted longitudinally into the stabilizer gap 10128 to join the foot connector 9164 to the body stabilizer 8112 on a second longitudinal stabilizer side 10130b of the body stabilizer 8112. The first stabilizer base 10124a, the second stabilizer base 10124b, and the stabilizer gap 10128 may be configured so that a press-fit and/or frictional engagement between the body stabilizer 8112 and the foot connector 8164 is created when the foot connector 8164 is inserted into the stabilizer gap 10128. The press-fit and/or frictional engagement between the body stabilizer 8112 and the foot connector 8164 may help prevent the foot connector 8164 from being undesirably detached from the body stabilizer 8112.

The first foot connector surface 8170a may also include at least one foot locking projection 8172 that is configured to engage at least one foot locking detent 10174 in the second stabilizer surface 8122b when the foot connector 8164 is joined to the body stabilizer 8112. The engagement between the foot locking projection 8172 and the foot locking detent 10174 may also help prevent the foot connector 8164 from being undesirably detached from the body stabilizer 8112.

As shown in FIGS. 8 and 11, the foot platform 8162 may include at least one foot aperture 8176 (shown here as foot apertures 8176a, 8176b, and 8176c) extending transversely therethrough. In the example configuration of FIGS. 8 and 11, the foot platform 8162 includes a first foot aperture 8176a laterally positioned along a first lateral foot side 8178a of the foot platform 8162. The foot platform 8162 also includes a second foot aperture 8176b laterally positioned along a second longitudinal foot side 8178b of the foot platform 8162. The first and second foot apertures 8176a, 8176b may be longitudinally aligned with one another or longitudinally offset from one another. A third foot aperture 8176c may extend transversely through the foot platform 8162 on a longitudinal side 8180 of the foot platform that is opposite the body stabilizer 8112 when the foot platform 8162 is connected to the body stabilizer 8112. The foot apertures 8176a, 8176b, 8176c may be used by at least one medical professional to grip the foot platform 8162 when transporting a restrained subject S.

The first foot surface 8166a may also include a foot handle 8182 projecting transversely from the first foot surface 8166a. The foot handle 8182 may include a foot handle aperture 8184 extending laterally through the foot handle 8182. The foot handle aperture 8184 may be used by at least one medical professional to grip the foot handle 8182 and the foot platform 8162 when transporting a restrained subject.

The restraint assembly 8110 may include at least one foot strap 8186 (shown here as foot straps 8186a and 8186b) configured to be selectively attached to the foot platform 8162 and selectively prevent the egress of the subject's feet FT and/or legs L from the foot platform 8162. The at least one foot strap 8186 may also be configured to immobilize at least a portion of the subject's feet FT and/or legs L on the foot platform 8162. The restraint assembly 8110 may include, for example, first and second foot straps 8186a, 8186b. The first foot strap 8186a may be looped through and extend between the first foot aperture 8176a and the foot handle aperture 8184 to restrain one leg L and foot FT of the subject S. The second foot strap 8186b may be looped through and extend between the second foot aperture 8176b and the foot handle aperture 8184 to restrain the other leg L and foot FT of the subject S.

Each of the first and second foot straps 8186a, 8186b may be selectively longitudinally movable along their respective foot apertures 8176a, 8176b and the foot handle aperture 8184. This longitudinal movement allows each of the first and second foot straps 8186a, 8186b to be placed in selected longitudinal positions along the subject's legs L and/or FT in order to correspond to the subject's anatomy. Each of the first and second foot straps 8186a, 8186b may be selectively retained attached to the foot platform 8162 and engaged to the subject's legs L and/or FT through the use of at least one of a clip, an adhesive, a hook and loop fastener, a magnetic fastener, a snap fastener, a hook and eye fastener, a button, a string and eye fastener, a saw tooth fastener, and/or any other suitable fastener or combination of fasteners.

The first foot surface 8166a may have a reusable or disposable foot cushion 8188 provided thereon that is configured to be interposed between the first foot surface 8166a and the subject's feet FT and/or legs L. The foot cushion 8188 provides a softer surface for the subject's feet FT and/or legs L to be laid on than the substantially rigid first foot surface 8166a. In the example configuration shown in FIG. 8, the foot cushion 8188 is a single cushion. Alternatively, the foot cushion 8188 may comprise a plurality of separate cushions. In such a configuration, one cushion may be interposed between the first foot surface 8166a and one leg/foot L/FT of the subject S, while a separate second cushion may be interposed between the first foot surface 8166a and the subject's other leg/foot. The foot cushion 8188 may be substantially permanently attached to the first foot surface 8166a or may be configured to be selectively removed from the first foot surface 8166a as desired.

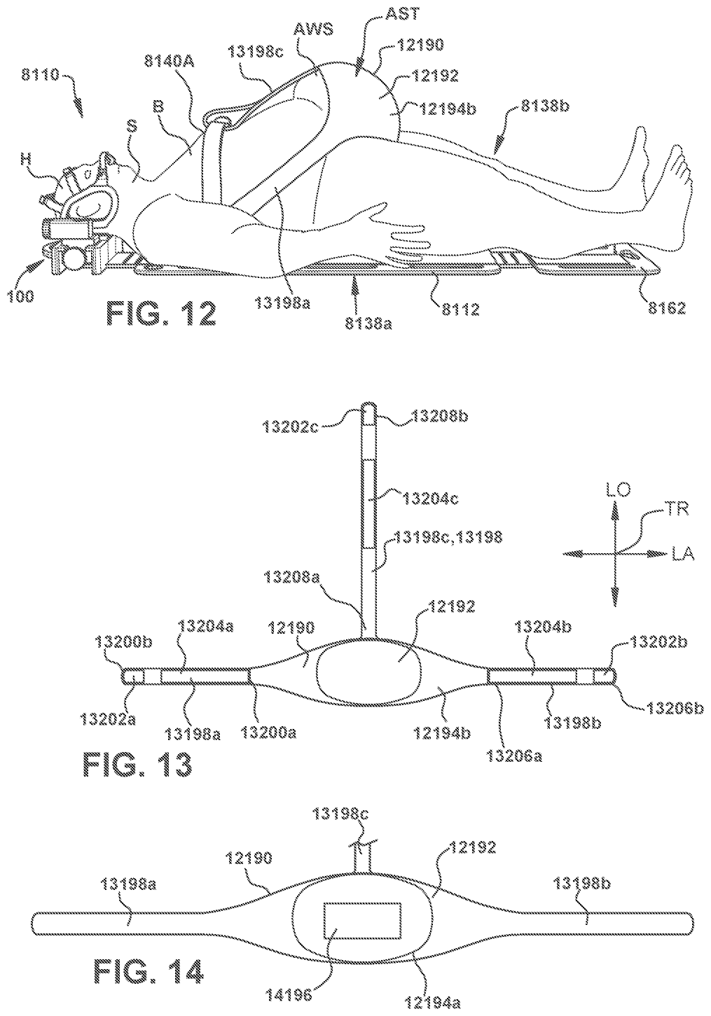

As shown in FIGS. 12-14, the restraint assembly 8110 may include an abdominal sling 12190. The abdominal sling 12190 includes a central sling portion 12192 having a first sling surface 12194a configured to selectively engage the subject's abdominal wall skin AWS, and a second sling surface 12194b configured to face away from the subject's abdominal wall skin AWS when the first sling surface 12194a is engaged to the subject S. The first sling surface 12194a may include a biocompatible adhesive 14196 provided thereon for selectively maintaining the first sling surface's engagement to the subject's abdominal wall skin AWS.

The abdominal sling 12190 also has at least one sling strap 13198 (shown here as sling straps 13198a, 13198b, and 13198c) extending from the central sling portion 12192. In the example configuration shown in FIGS. 12-14, the abdominal sling 12190 includes first, second, and third sling straps 13198a, 13198b, 13198c extending from the central sling portion 12192. The first sling strap 13198a has a first end 13200a connected to the central sling portion 12192. A second end 13200b of the first sling strap 13198a may be selectively looped around the first restraining strap 8140a adjacent the first lateral stabilizer side 8138a and pulled toward the central sling portion 12192 to tension the first sling strap 13198a. Once the first sling strap 13198a is tensioned as desired, the second end 13200b of the first sling strap 13198a may be selectively attached to another portion of the first sling strap 13198a to maintain the desired tensioning in the first sling strap 13198a. The second end 13200b of the first sling strap 13198a may be attached to the other portion of the first sling strap 13198a via at least one of a clip, an adhesive, a hook and loop fastener, a magnetic fastener, a snap fastener, a hook and eye fastener, a button, a string and eye fastener, a saw tooth fastener, and/or any other suitable fastener or combination of fasteners. As shown in FIG. 13, the second end 13200b of the first sling strap 13198a may include a first hook portion 13202a that is configured to be connected to a first loop portion 13204a on the first sling strap 13198a to attach the second end 13200b of the first sling strap 13198a to the remainder of the first sling strap 13198a.

As shown in FIGS. 12-14, the second sling strap 13198b has a first end 13206a connected to the central sling portion 12192. A second end 13206b of the second sling strap 13198b may be selectively looped around the first restraining strap 8140a adjacent the second lateral stabilizer side 8138b and pulled toward the central sling portion 12192 to tension the second sling strap 13198b. Once the second sling strap 13198b is tensioned as desired, the second end 13206b of the second sling strap 13198b may be selectively attached to another portion of the second sling strap 13198b to maintain the desired tensioning in the second sling strap 13198b. The second end 13206b of the second sling strap 13198b may be attached to the other portion of the second sling strap 13198b via at least one of a clip, an adhesive, a hook and loop fastener, a magnetic fastener, a snap fastener, a hook and eye fastener, a button, a string and eye fastener, a saw tooth fastener, and/or any other suitable fastener or combination of fasteners. As shown in FIG. 13, the second end 13206b of the second sling strap 13198b may include a second hook portion 13202b that is configured to be connected to a second loop portion 13204b on the second sling strap 13198b to attach the second end 13206b of the second sling strap 13198b to the remainder of the second sling strap 13198b.

As shown in FIGS. 12-13, the third sling strap 13198c has a first end 13208a connected to the central sling portion 12192. The third sling strap 13198c may be positioned laterally between the first and second sling straps 13198a, 13198b. Therefore, a second end 13208b of the third sling strap 13198c may be selectively looped around the first restraining strap 8140a between the first and second lateral stabilizer sides 8138a, 8138b and pulled toward the central sling portion 12192 to tension the third sling strap 13198c. Once the third sling strap 13198c is tensioned as desired, the second end 13208b of the third sling strap 13198c may be selectively attached to another portion of the third sling strap 13198c to maintain the desired tensioning in the third sling strap 13198c. The second end 13208b of the third sling strap 13198c may be attached to the other portion of the third sling strap 13198c via at least one of a clip, an adhesive, a hook and loop fastener, a magnetic fastener, a snap fastener, a hook and eye fastener, a button, a string and eye fastener, a saw tooth fastener, and/or any other suitable fastener or combination of fasteners. As shown in FIG. 13, the second end 13208b of the third sling strap 13198c may include a third hook portion 13202c that is configured to be connected to a third loop portion 13204c on the third sling strap 13198c to attach the second end 13208b of the third sling strap 13198c to the remainder of the third sling strap 13198c.

When the central sling portion 12192 is engaged to the subject's abdominal wall skin AWS, the tensioning in at least one of the first, second, and third sling straps 13198a, 13198b, 13198c is configured to cause the central sling portion 12192 to exert a force on the subject's abdominal wall skin AWS to urge the subject's abdominal wall soft tissue AST cephalad. The term “cephalad” is defined herein as toward the head H of the subject S. The first, second, and third sling straps 13198a, 13198b, 13198c may thus be tensioned as desired to responsively urge the subject's abdominal wall soft tissue AST cephalad as desired. Urging the subject's abdominal wall soft tissue AST cephalad can be especially helpful when it is appropriate for a medical professional to access portions of the subject S that would have been otherwise blocked or covered by the subject's abdominal wall soft tissue AST.

Although the first, second, and third sling straps 13198a, 13198b, 13198c are attached to the body stabilizer 8112 through the first restraining strap 8140a, at least one of the first, second, and third sling straps 13198a, 13198b, 13198c may be directly connected to the body stabilizer 8112 such as, for example, by being looped through at least one of the stabilizer apertures 8136a, 8136b, 8136c, 8136d, 8136e, 8136f. For example, the first sling strap 13198a may be connected to body stabilizer 8112 by being looped through the first stabilizer aperture 8136a on the first lateral stabilizer side 8138a, the second sling strap 13198b may be connected to body stabilizer 8112 by being looped through the fourth stabilizer aperture 8136d on the second lateral stabilizer side 8138b, and the third sling strap 13198c may be looped around the first restraining strap 8140a as described above. Alternatively or additionally, at least one of the first, second, and third restraining straps 13198a, 13198b, 13198c may be connected to at least one of the second and third restraining straps 8140b, 8140c instead of, or in addition to, being connected to the first restraining strap 8140a.

At least one of the central sling portion 12192, the first sling strap 13198a, the second sling strap 13198b, and the third sling strap 13198c may be formed separately from and then subsequently connected to at least one other of the central sling portion 12192, the first sling strap 13198a, the second sling strap 13198b, and the third sling strap 13198c. Alternatively or additionally, at least one of the central sling portion 12192, the first sling strap 13198a, the second sling strap 13198b, and the third sling strap 13198c may be integrally formed with at least one other of the central sling portion 12192, the first sling strap 13198a, the second sling strap 13198b, and the third sling strap 13198c as a single-piece, unitary construct.

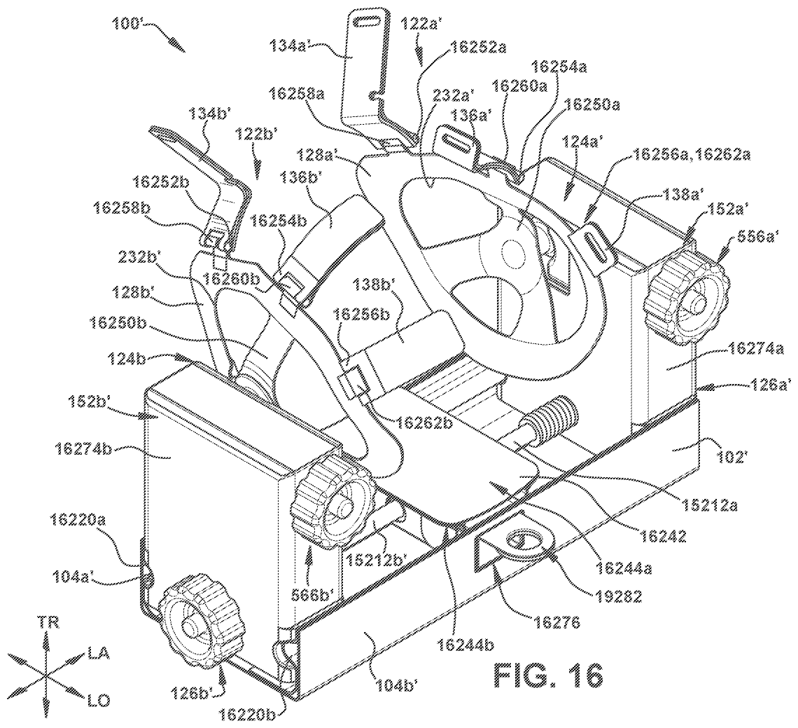

FIGS. 16-27 illustrate a second embodiment of a head restraint assembly 100′ that may be included as part of the restraint assembly 8110. The head restraint assembly 100′ of FIGS. 16-27 is similar to the head restraint assembly 100 of FIGS. 1-12 and 15 and therefore, structures of FIGS. 16-27 that are the same as or similar to those described with reference to FIGS. 1-12 and 15 have the same reference numbers with the addition of a “prime” mark. Description of common elements and operation similar to those in the previously described first embodiment may not be repeated with respect to the second embodiment, but should instead be considered to be incorporated below by reference as appropriate. Furthermore, elements and functionality shown or described with respect to one embodiment may be shared by any of the other embodiments whether or not expressly stated or shown.

As shown in FIGS. 16-18, the head restraint assembly 100′ includes a restraint platform 102′. The restraint platform 102′ has a first platform portion 104a′ and a second platform portion 104b′ that is separated from the first platform portion 104a′ by a longitudinally extending restraint gap 108′. The first platform portion 104a′ includes a transversely extending first column 110a′ and a first rail 16220a extending laterally from the first column 110a′ toward the second platform portion 104b′. The second platform portion 104b′ includes a longitudinally extending second column 110b′ and a second rail 16220b extending laterally from the second column 110b′ toward the first platform portion 104a′. The first and second rails 16220a, 16220b extend toward one another and, thus, a portion of the restraint gap 108′ that extends between the first and second flanges 112a′, 112b′ may be longitudinally smaller than a portion of the restraint gap 108′ that extends between the first and second columns 110a′, 110b′. At least one of the first and second rails 16220a, 16220b may be formed integrally with a respective one of the first and second columns 110a′, 110b′ as a single-piece, unitary construct, or formed separately and subsequently attached to a respective one of the first and second columns 110a′, 110b′.

A third platform portion 104c′ of the restraint platform 102′ extends longitudinally between the first and second platform portions 104a′, 104b′ and connects the first and second platform portions 104a′, 104b′ to one another. At least one of the first, second, and third platform portions 104a′, 104b′, 104c′ may be formed separately from, and then subsequently connected to, at least one other of the first, second, and third platform portions 104a′, 104b′, 104c′. Alternatively or additionally, at least one of the first, second, and third platform portions 104a′, 104b′, 104c′ may be integrally formed with at least one other of the first, second, and third platform portions 104a′, 104b′, 104c′ as a single-piece, unitary construct.

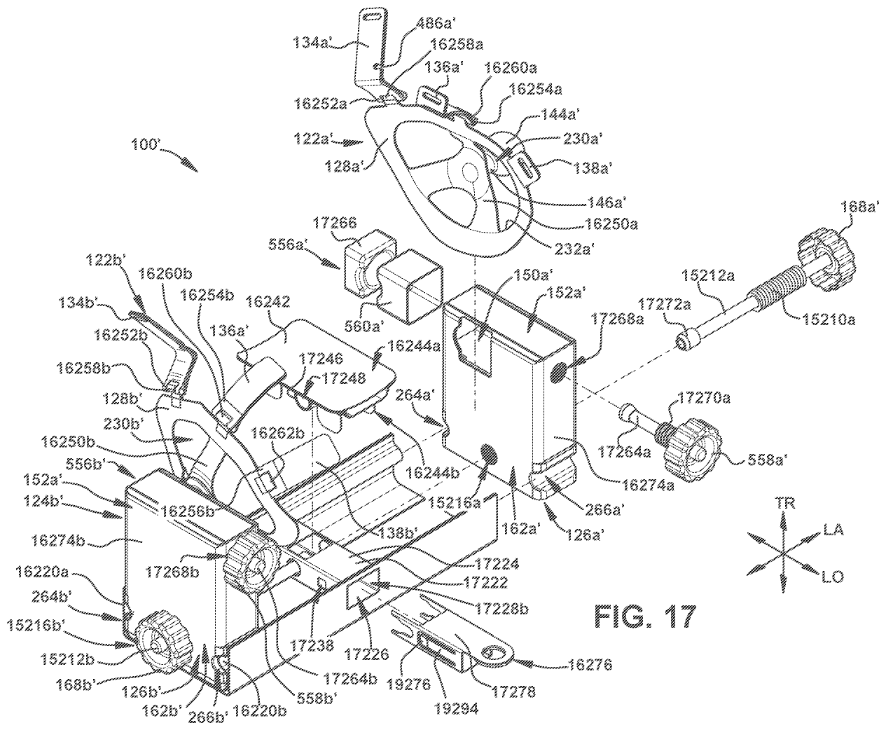

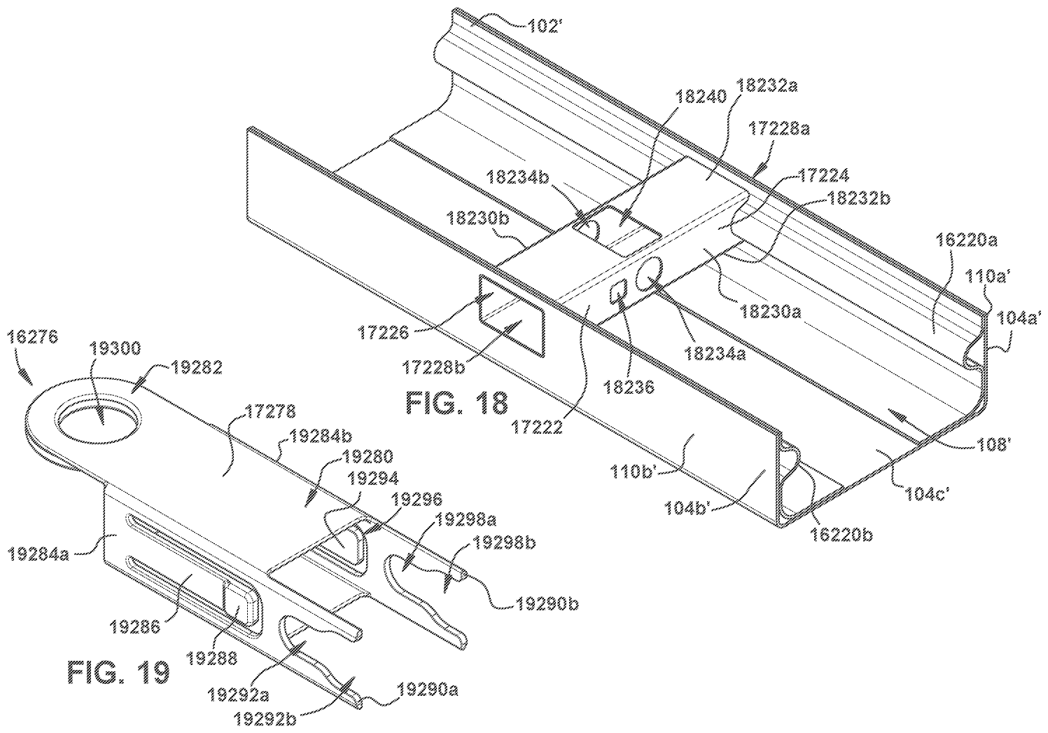

As shown in FIGS. 17-18, the restraint platform 102′ also includes a platform attachment member 17222. The platform attachment member 17222 includes a platform attachment body 17224 that extends longitudinally between the first and second platform portions 104a′, 104b′ and defines an inner platform attachment cavity 17226. The platform attachment cavity 17226 extends longitudinally between a first opening 17228a in the first column 110a′ and a second opening 17228b in the second column 110b′. Alternatively, the platform attachment cavity 17226 may extend longitudinally from the second column opening 17228b to a blind end adjacent the first platform portion 104a′ in configurations of the restraint platform 102′ where the first opening 17228a is omitted.

The platform attachment body 17224 includes first and second attachment body sidewalls 18230a, 18230b and first and second attachment body end walls 18232a, 18230b. The first attachment body sidewall 18230a includes a first drive shaft opening 18234a and a holding opening 18236 laterally extending therethrough. Similarly, the second attachment body sidewall 18230b includes a second drive shaft opening 18234b and a locking opening 17238 extending therethrough. As shown in FIGS. 17-18, the holding and locking openings 18236, 17238 are longitudinally offset from one another such that the locking opening 17238 is longitudinally spaced closer to the second platform portion 104b′ than the holding opening 18236, while the first and second draft shaft openings 18234a, 18234b are longitudinally aligned with one another. The first attachment body end wall 18232a includes a head platform opening 18240 extending transversely therethrough.

As shown in FIGS. 16-17, the head restraint assembly 100′ includes a head platform 16242 that is separate from, and selectively connectable to, the restraint platform 102′. The head platform 16242 includes a first platform surface 16244a configured to have the subject's head H laid thereon. The first platform surface 16244a may be substantially arcuate in the lateral direction in order to accommodate the subject's head H. However, it is contemplated that the first platform surface 16244a may be curved in any desired manner or extend substantially flatly in the lateral direction as desired to accommodate the subject's head H. The first platform surface 16244a may have a reusable or disposable base cushion (e.g., the base cushion 114), of any desired material and construction, provided thereon that is configured to be interposed between the subject's head H and the first platform surface 16244a. The base cushion may be substantially permanently attached to the first platform surface 16244a or may be configured to be selectively removed from the first platform surface 16244a as desired.

As shown in FIG. 17, the head platform 16242 includes a second platform surface 16244b that is transversely opposite to the first platform surface 16244a. The second platform surface 16244b includes a head platform projection 17246 extending transversely therefrom. The head platform projection 17246 defines a cylindrical drive shaft tunnel 17248 that extends laterally therethrough. As shown in FIG. 16, when the head platform 16242 is selectively joined to the restraint platform 102′, the head platform projection 17246 is received in the head platform opening 18240 such that the drive shaft tunnel 17248 is longitudinally aligned with both the first and second drive shaft openings 18234a, 18234b. The drive shaft tunnel 17248 may include a central partition that at least partially separates the drive shaft tunnel 17248 into first and second tunnel portions 17248a, 17248b.

As shown in FIGS. 16-17, the first lateral platform side 120a′ of the restraint platform 102′ may include at least one of a first head restraint 122a′, a first articulating assembly 124a′, and a first sliding member 126a′.

The first head restraint 122a′ is configured to selectively engage the first side FS of the subject's head H. The first head restraint 122a′ is substantially the same as the first head restraint 122a of FIGS. 1-12 and 15. However, the first head restraint 122a′ includes a first support webbing 16250a for reinforcing the structure of the first base portion 128a′. The first support webbing 16250a is aligned with the first earhole 230a′ and extends transversely from the inner periphery 232a′ of the first base portion 128a′.

The first head restraint 122a′ includes the first cheekbone flange 134a′, the first forehead flange 136a′, and the first crown flange 138a′, though the first head restraint 122a′ may have any desired number of flanges. Each of the first cheekbone flange 134a′, the first forehead flange 136a′, and the first crown flange 138a′ are formed separately from, and then subsequently connected to, the first base portion 128a′, such as to, for example, the outer periphery 140a′ of the first base portion 128a′.

The first cheekbone flange 134a′, the first forehead flange 136a′, and the first crown flange 138a′ may include a first cheekbone flange attachment member 16252a, a first forehead flange attachment member 16254a, and a first crown flange attachment member 16256a, respectively, for selectively connecting each of the first cheekbone flange 134a′, the first forehead flange 136a′, and the first crown flange 138a′ to the first base portion 128a′. In particular, the first cheekbone flange attachment member 16252a may be selectively connected to a first cheekbone flange connector member 16258a of the first base portion 128a′, the first forehead flange attachment member 16254a may be selectively connected to a first forehead flange connector member 16260a of the first base portion 128a′, and the first crown flange attachment member 16256a may be selectively connected to a first crown flange connector member 16262a of the first base portion 128a′.

In the configuration shown in FIGS. 16-17, the first cheekbone flange connector member 16258a, the first forehead flange connector member 16260a, and the first crown flange connector member 16262a are each in the form of a hook, while the first cheekbone flange attachment member 16252a, the first forehead flange attachment member 16254a, and the first crown flange attachment member 16256a are each in the form of a bar to which the hooks may be selectively attached. However, each of the flange attachment members 16252a, 16254a, 16256a may be connected to the flange connector members 16258a, 16260a, 16262a in any other desired manner, such as, for example, via the use of at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

The first cheekbone flange connector member 16258a, the first forehead flange connector member 16260a, and the first crown flange connector member 16262a may extend from the outer periphery 140a′ of the first base portion 128a′ or from any other portion of the first base portion 128a′. Via the hook-bar engagement, each of the first cheekbone flange 134a′, the first forehead flange 136a′, and the first crown flange 138a′ may selectively pivot relative to the first base portion 128a′ when connected thereto. This pivoting functionality permits at least one of the first cheekbone flange 134a′, the first forehead flange 136a′, and the first crown flange 138a′ to be selectively positioned to a desired orientation relative to the subject's head H and/or to adapt to the anatomy of the subject's head H.

The first articulating assembly 124a′ may be substantially the same as the first articulating assembly 124a of FIGS. 1-5. Therefore, the first articulating assembly 124a′ may be in the form of a first ball-and-socket assembly 124a′. The first ball-and-socket assembly 124a′ may include the first ball portion 144a′ attached to the first ball stem 146a′. The first ball stem 146a′ connects the first ball portion 144a′ to the first head restraint 122a′ (e.g., to the first support webbing 16250a). At least one of the first ball portion 144a′, the first ball stem 146a′, and the first support webbing 16250a may be formed separately from and then subsequently connected to at least one other of the first ball portion 144a′, the first ball stem 146a′, and the first support webbing 16250a. Alternatively or additionally, at least one of the first ball portion 144a′, the first ball stem 146a′, and the first support webbing 16250a may be integrally formed with at least one other of the first ball portion 144a′, the first ball stem 146a′, and the first support webbing 16250a as a single-piece, unitary construct.

The first ball-and-socket assembly 124a′ also includes a first socket portion 150a′ associated with, such as by being recessed into, a first ball-and-socket base 152a′. The first ball portion 144a′ is selectively received in the first socket portion 150a′ and is selectively articulatable relative to the first socket portion 150a′. This arrangement provides the first head restraint 122a′ with at least three degrees of freedom of movement for the first head restraint 122a′ to conform to the first side FS of the subject's head H.

As shown in FIG. 17, the first ball-and-socket assembly 124a′ may include the first ball inhibiting mechanism 556a′ for selectively retaining the first head restraint 122a′ in a fixed position. The first ball inhibiting mechanism 556a′ may include a first inhibiting shaft 17264a, a first inhibiting control knob 558a′ connected to one end of the first inhibiting shaft 17264a, a first moveable ball stop 560a′ connected to another end of the first inhibiting shaft 17264a, and a first fixed ball stop 17266a fixed in positioned in the first socket portion 150a′. One end of the first inhibiting shaft 17264a is connected to the first moveable ball stop 560a′, while the other end of the first inhibiting shaft 17264a is connected to the first inhibiting control knob 558a′. The first ball-and-socket base 152a′ includes a first threaded opening 17268a in fluid communication with the first socket portion 150a′. The first inhibiting shaft 17264a extends through the first threaded opening 17268a such that the first moveable ball stop 560a′ is inside the first socket portion 150a′, the first inhibiting control knob 558a′ is outside the first ball-and-socket base 152a′, and a first threaded portion 17270a of the first inhibiting shaft 17264a is threadably engaged in the first threaded opening 17268a.

When desired, the first inhibiting control knob 558a′ may be actuated, such as by being rotated in a first direction, to responsively cause the first moveable ball stop 560a′ to move in the longitudinal direction toward the first ball portion 144a′ and the first fixed ball stop 17266a. The first moveable ball stop 560a′ may be moved longitudinally until the first moveable ball stop 560a′ engages the first ball portion 144a′. Movement of the first ball portion 144a′ (and the first ball stem 146a′ connected thereto) relative to the first ball-and-socket base 152a′ is substantially inhibited once the first moveable ball stop 560a′ engages the first ball portion 144a′. Because the first head restraint 122a′ is connected to the first ball portion 144a′ via the first ball stem 146a′, inhibiting the movement of the first ball portion 144a′ causes the first head restraint 122a′ to be retained in a fixed position. Therefore, the first head restraint 122a′ may be moved to a desired position in which the first head restraint 122a′ conforms to the first side FS of the subject's head H, and then locked in place by the first ball inhibiting mechanism 556a′ once the desired position is reached. When movement of the first head restraint 122a′ is once again desired, the moveable first ball stop 560a′ may be disengaged from the first ball portion 144a′ by actuating the first inhibiting control knob 558a′, such as by rotating the first inhibiting control knob 558a′ in a second direction, opposite the first direction, to move the first moveable ball stop 560a′ longitudinally away from the first ball portion 144a′ and the first fixed ball stop 17266a.

As shown in FIGS. 16-17, the first sliding mechanism 126a′ includes a first sliding base 162a′. The first sliding base 162a′ includes first and second sliding base notches 264a′, 266a′ that extend longitudinally inward from an exterior of the first sliding base 162a′. The first sliding base notch 264a′ is configured to receive the first rail 16220a, while the second sliding base notch 266a′ is configured to receive the second rail 16220b. As shown in FIG. 17, the first sliding mechanism 126a′ also includes a first drive shaft 15212a, a first sliding control knob 168a′ connected to one end of the first drive shaft 15212a, a first end support 17272a at an opposite end of the first drive shaft 15212a, and a first threaded portion 15210a′ laterally between the ends of the first drive shaft 15212a. The first drive shaft 15212a extends laterally through a first threaded opening 15216a′ in the first sliding base 162a′ such that the first threaded portion 15210a′ is threadably engaged in the first threaded opening 15216a′. The first end support 17272a of the first drive shaft 15212a is inserted through the first drive shaft opening 18234a into the drive shaft tunnel 17248 (e.g., into the first tunnel portion 17248a). The first drive shaft 15212a thus is supported via the first threaded portion 15210a when the first threaded portion 15210a is threadably engaged in the first threaded opening 15216a′, and via the first end support 17272a when the first end support 17272a is received in the drive shaft tunnel 17248.

When desired, the first sliding control knob 168a′ may be actuated, such as by being rotated, to responsively cause the first drive shaft 15212a to rotate relative to the first sliding base 162a′. The rotating first drive shaft 15212a responsively causes the first sliding base 162a′ to move laterally along the first threaded portion 15210a′ and to concurrently slide laterally along the first and second rails 16220a, 16220b of the restraint platform 102′. Therefore, through actuation of the first sliding control knob 168a′, the first sliding base 162a′ may be selectively moved laterally toward and away from the second lateral platform side 120b′ of the restraint platform 102′.

As shown in FIGS. 16-17, the first sliding base 162a′ and the first ball-and-socket base 152a′ may be two portions of a single first base housing 16274a. Therefore, the first sliding base 162a′ is fixed to the first ball-and-socket base 152a′ and operably connected to the first head restraint 122a′ through the first ball-and-socket assembly 124a′. The first head restraint 122a′ thus is connected to the first lateral platform side 120a′ of the restraint platform 102′ through the first ball-and-socket assembly 124a′ and the first sliding mechanism 126a′. Selective lateral movement of the first sliding base 162a′ responsively causes the first head restraint 122a′ (and thus the first base housing 16274a as a whole) to move laterally relative to the restraint platform 102′. Accordingly, the first sliding control knob 558a′ may be selectively actuated to cause the first head restraint 122a′ to move laterally toward a subject's head H received on the first platform surface 16244a to engage the first side FS of the subject's head H, and selectively actuated to cause the first head restraint 122a′ to move laterally away the subject's head H to disengage the first side FS of the subject's head H.

The second lateral platform side 120b′ of the restraint platform 102′ includes at least one of a second head restraint 122b′, a second articulating assembly 124b′, and a second sliding member 126b′.

The second head restraint 122b′ is configured to selectively engage a second side SS of the subject's head H, which is laterally opposite the first side FS of the subject's head H. The second head restraint 122b′ is substantially the same as the second head restraint 122b of FIGS. 1-12 and 15. However, the second head restraint 122b′ includes a second support webbing 16250b for reinforcing the structure of the second base portion 128b′. The second support webbing 16250b is aligned with the second earhole 230b′ and extends transversely from the inner periphery 232b′ of the second base portion 128b′.

The second head restraint 122b′ includes the second cheekbone flange 134b′, the second forehead flange 136b′, and the second crown flange 138b′, though the second head restraint 122b′ may have any number of flanges. Each of the second cheekbone flange 134b′, the second forehead flange 136b′, and the second crown flange 138b′ are formed separately from, and then subsequently connected to, the second base portion 128b′, such as to, for example, the outer periphery 140b′ of the second base portion 128b′.

The second cheekbone flange 134b′, the second forehead flange 136b′, and the second crown flange 138b′ may include a second cheekbone flange attachment member 16252b, a second forehead flange attachment member 16254b, and a second crown flange attachment member 16256b, respectively, for selectively connecting each of the second cheekbone flange 134b′, the second forehead flange 136b′, and the second crown flange 138b′ to the second base portion 128b′. In particular, the second cheekbone flange attachment member 16252b may be selectively connected to a second cheekbone flange connector member 16258b of the second base portion 128b′, the second forehead flange attachment member 16254b may be selectively connected to a second forehead flange connector member 16260b of the second base portion 128b′, and the second first crown flange attachment member 16256b may be selectively connected to a second crown flange connector member 16262b of the second base portion 128b′.

In the configuration shown in FIGS. 16-17, the second cheekbone flange connector member 16258b, the second forehead flange connector member 16260b, and the second crown flange connector member 16262b are each in the form of a hook, while the second cheekbone flange attachment member 16252b, the second forehead flange attachment member 16254b, and the second crown flange attachment member 16256b are each in the form of a bar to which the hooks may be selectively attached. However, each of the flange attachment members 16252b, 16254b, 16256b may be connected to the flange connector members 16258b, 16260b, 16262b in any other desired manner, such as, for example, via the use of at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

The second cheekbone flange connector member 16258b, the second forehead flange connector member 16260b, and the second crown flange connector member 16262b may extend from the outer periphery 140b′ of the second base portion 128b′ or from any other portion of the second base portion 128b′. Via the hook-bar engagement, each of the second cheekbone flange 134b′, the second forehead flange 136b′, and the second crown flange 138b′ may selectively pivot relative to the second base portion 128b′ when connected thereto. This pivoting functionality permits at least one of the second cheekbone flange 134b′, the second forehead flange 136b′, and the second crown flange 138b′ to be selectively positioned to a desired orientation relative to the subject's head H and/or to adapt to the anatomy of the subject's head H.

The second articulating assembly 124b′ may be substantially the same as the second articulating assembly 124b of FIGS. 1-5. Therefore, the second articulating assembly 124b′ may be in the form of a second ball-and-socket assembly 124b′. The second ball-and-socket assembly 124b′ may include the second ball portion 144b′ attached to the second ball stem 146b′. The second ball stem 146b′ connects the second ball portion 144b′ to the second head restraint 122b′ (e.g., to the second support webbing 16250b). At least one of the second ball portion 144b′, the second ball stem 146b′, and the second support webbing 16250b may be formed separately from, and then subsequently connected to, at least one other of the second ball portion 144b′, the second ball stem 146b′, and the second support webbing 16250b. Alternatively or additionally, at least one of the second ball portion 144b′, the second ball stem 146b′, and the second support webbing 16250b may be integrally formed with at least one other of the second ball portion 144b′, the second ball stem 146b′, and the second support webbing 16250b as a single-piece, unitary construct.

The second ball-and-socket assembly 124b′ also includes a second socket portion 150b′ associated with, such as by being recessed into, a second ball-and-socket base 152b′. The second ball portion 144b′ is selectively received in the second socket portion 150b′ and is selectively articulatable relative to the second socket portion 150b′. This arrangement provides the second head restraint 122b′ with at least three degrees of freedom of movement for the second head restraint 122b′ to conform to the second side SS of the subject's head H.

The second ball-and-socket assembly 124b′ may include a second ball inhibiting mechanism 556b′ for selectively retaining the second head restraint 122b′ in a fixed position. The second ball inhibiting mechanism 556b′ may mirror the first ball inhibiting mechanism 556a′ shown in detail in FIG. 17. The second ball inhibiting mechanism 556b′ may include a second inhibiting shaft 17264b, a second inhibiting control knob 558b′ connected to one end of the second inhibiting shaft 17264b, a second moveable ball stop 560b′ connected to another end of the second inhibiting shaft 17264b, and a second fixed ball stop 17266b positioned in the second socket portion 150b′. One end of the second inhibiting shaft 17264b is connected to the second moveable ball stop 560b′, while the other end of the second inhibiting shaft 17264b is connected to the second inhibiting control knob 558b′. The second ball-and-socket base 152b′ includes a second threaded opening 17268b in fluid communication with the second socket portion 150b′. The second inhibiting shaft 17264b extends through the second threaded opening 17268b such that the second moveable ball stop 560b′ is inside the second socket portion 150b′, the second inhibiting control knob 558a′ is outside the second ball-and-socket base 152b′, and a second threaded portion 17270b of the second inhibiting shaft 17264b is threadably engaged in the second threaded opening 17268b.

When desired, the second inhibiting control knob 558b′ may be actuated, such as by being rotated in a first direction, to responsively cause the second moveable ball stop 560b′ to move in the longitudinal direction toward the second ball portion 144b′ and the second fixed ball stop 17266b. The second moveable ball stop 560b′ may be moved longitudinally until the second moveable ball stop 560b′ engages the second ball portion 144b′. Movement of the second ball portion 144b′ (and the second ball stem 146b′ connected thereto) relative to the second ball-and-socket base 152b′ is substantially inhibited once the second moveable ball stop 560b′ engages the second ball portion 144b′. Because the second head restraint 122b′ is connected to the second ball portion 144b′ via the second ball stem 146b′, inhibiting the movement of the second ball portion 144b′ causes the second head restraint 122b′ to be retained in a fixed position. Therefore, the second head restraint 122b′ may be moved to a desired position in which the first head restraint 122b′ conforms to the second side SS of the subject's head H, and then locked in place by the second ball inhibiting mechanism 556b′ once the desired position is reached. When movement of the second head restraint 122b′ is once again desired, the moveable second ball stop 560b′ may be disengaged from the second ball portion 144b′ by actuating the second inhibiting control knob 558b′, such as by rotating the second inhibiting control knob 558b′ in a second direction opposite the first direction, to move the second moveable ball stop 560b′ longitudinally away from the second ball portion 144b′ and the second fixed ball stop 17266b.

As shown in FIGS. 16-17, the second sliding mechanism 126b′ includes a second sliding base 162b′. The second sliding mechanism 126b′ may mirror the first sliding mechanism 126a′ shown in detail in FIG. 17. The second sliding base 162b′ includes first and second sliding base notches 264b′, 266b′ that extend longitudinally inward from an exterior of the second sliding base 162b′. The first sliding base notch 264b′ is configured to receive the first rail 16220a, while the second sliding base notch 266b′ is configured to receive the second rail 16220b. The second sliding mechanism 126b′ also includes a second drive shaft 15212b, a second sliding control knob 168b′ connected to one end of the second drive shaft 15212b, a second end support 17272b at an opposite end of the second drive shaft 15212b, and a second threaded portion 15210b′ laterally between the ends of the second drive shaft 15212b. The second drive shaft 15212b extends laterally through a second threaded opening 15216b′ in the second sliding base 162b′ such that the second threaded portion 15210b′ is threadably engaged in the second threaded opening 15216b′. The second end support 17272b of the second drive shaft 15212b is inserted through the second drive shaft opening 18234b into the drive shaft tunnel 17248 (e.g., into the second tunnel portion 17248b). The second drive shaft 15212b thus is supported via the second threaded portion 15210b when the second threaded portion 15210b is threadably engaged in the second threaded opening 15216b′, and via the second end support 17272b when the second end support 17272b is received in the drive shaft tunnel 17248. The first and second drive shafts 15212a, 15212b extending into the drive shaft tunnel 17248 at least partially selectively prevents the head platform 16242 from being undesirably disconnected from the restraint platform 102′.

When desired, the second sliding control knob 168b′ may be actuated, such as by being rotated, to responsively cause the second drive shaft 15212b to rotate relative to the second sliding base 162b′. The rotating second drive shaft 15212b responsively causes the second sliding base 162b′ to both move laterally along second first threaded portion 15210b′ and to concurrently slide laterally along the first and second rails 16220a, 16220b of the restraint platform 102′. Therefore, through actuation of the second sliding control knob 168b′, the second sliding base 162b′ may be selectively moved laterally toward and away from the first lateral platform side 120a′ of the restraint platform 102′. Because the second drive shaft 15212b is separate from the first drive shaft 15212a, the first and second sliding bases 162a′, 162b′ may be moved relative to one another such that one of the first and second sliding bases 162a′, 162b′ may be moved laterally while the other remains stationary.

As shown in FIGS. 16-17, the second sliding base 162b′ and the second ball-and-socket base 152b′ may be two portions of a single second base housing 16274b. Therefore, the second sliding base 162b′ is fixed to the second ball-and-socket base 152b′ and operably connected to the second head restraint 122b′ through the second ball-and-socket assembly 124b′. The second head restraint 122b′ thus is connected to the second lateral platform side 120b′ of the restraint platform 102′ through the second ball-and-socket assembly 124b′ and the second sliding mechanism 126b′. Selective lateral movement of the second sliding base 162b′ responsively causes the second head restraint 122b′ (and thus the second base housing 16274b as a whole) to move laterally relative to the restraint platform 102′. Accordingly, the second sliding control knob 558b′ may be selectively actuated to cause the second head restraint 122b′ to move laterally toward a subject's head H received on the first platform surface 16244a to engage the second side SS of the subject's head H, and selectively actuated to cause the second head restraint 122b′ to move laterally away from the subject's head H to disengage from the second side SS of the subject's head H.

Via the first and second sliding mechanisms 126a′, 126b′, the first and second head restraints 122a′, 122b′ may also be selectively movable in the lateral direction relative to the restraint platform 102′. Furthermore, the first and second ball-and-socket assemblies 124a′, 124b′ permit the first and second head restraints 122a′, 122b′ to articulate relative to one another. Therefore, the first and second head restraints 122a′, 122b′ may be adjustable relative to the restraint platform 102′ and the subject's head H when received thereon and may be at least partially independently adjustable relative to one another in order to conform to their respective sides FS, SS of the subject's head H.

As shown in FIGS. 16-17, the head restraint assembly 100′ also includes a quick release mechanism 16276. As shown in FIGS. 17 and 19-20, the quick release mechanism 16276 comprises a release body 17278 that is selectively at least partially inserted into the platform attachment member 17222. In particular, the release body 17278 is configured to be at least partially inserted through the second opening 17228b of the second column 110b′ into the platform attachment cavity 17266.

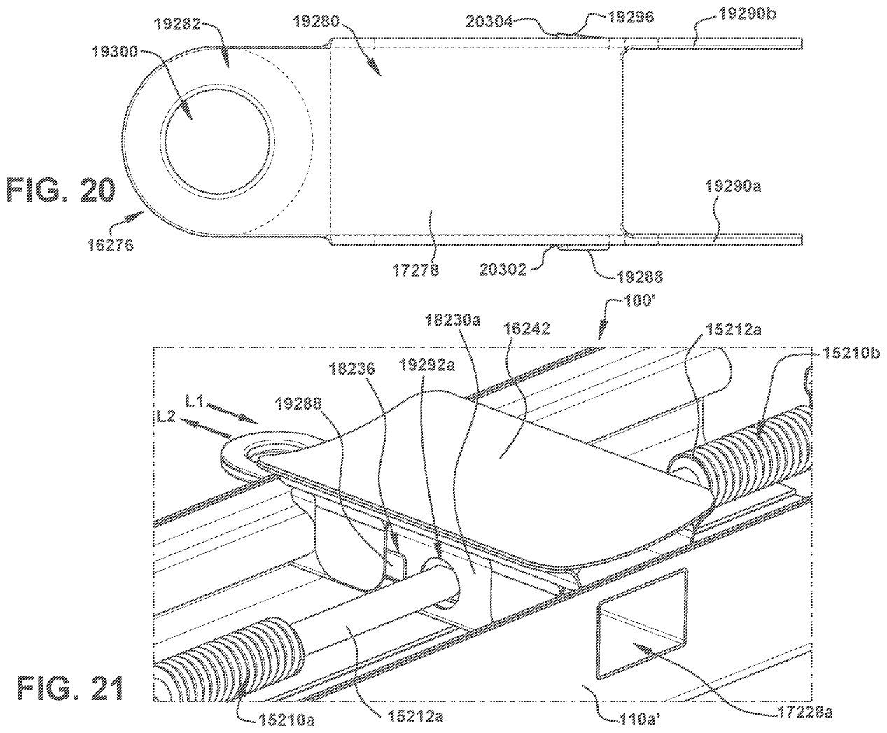

As shown in FIGS. 19-20, the release body 17278 includes an insertion portion 19280 and a gripping portion 19282. The insertion portion 19280 includes laterally opposing first and second release sidewalls 19284a, 19284b. The first release sidewall 19284a has a holding prong 19286 formed therein. The holding prong 19286 is selectively elastically deflectable in the lateral direction relative to the remainder of the first release sidewall 19284a. The holding prong 19286 includes a holding tab 19288 extending laterally therefrom. The first release sidewall 19284a also defines a first release claw 19290a. The first release claw 19290a has longitudinally adjacent first and second claw portions 19292a, 19292b. As shown in FIG. 19, an inner transverse height of the first claw portion 19292a is smaller than the inner transverse height of the second claw portion 19292b. The first claw portion 19292a is also spaced closer to the holding prong 19286 than the second claw portion 19292b.

As shown in FIGS. 19-20, the second release sidewall 19284b has a locking prong 19294 formed therein. The locking prong 19294 is selectively deflectable in the lateral direction relative to the remainder of the second release sidewall 19284b. The locking prong 19294 includes a locking tab 19296 extending laterally therefrom. The second release sidewall 19284b also defines a second release claw 19290n. The second release claw 19290b has longitudinally adjacent first and second claw portions 19298a, 19298b. As shown in FIG. 19, an inner transverse height of the first claw portion 19298a is smaller than the inner transverse height of the second claw portion 19298b. The first claw portion 19298a is also spaced closer to the locking prong 19294 than the second claw portion 19298b.

As shown in FIGS. 19-20, the gripping portion 19282 of the release body 17278 includes a gripping aperture 19300. FIG. 16 depicts the release body 17278 inserted into the platform attachment cavity 17266. As depicted in these Figures, although the insertion portion 19280 is positioned within the platform attachment cavity 17266, the gripping portion 19282 remains outside of the platform attachment cavity 17266 so that the medical professional may selectively manipulate the release body 17278 via the gripping portion 19282.

The holding and locking prongs 19286, 19294 may be elastically deflected laterally inward (e.g., toward one another) via contact between the holding and locking tabs 19288, 19296 and the first and second attachment body sidewalls 18230a, 18230b as the release body 17278 is inserted in a longitudinal holding direction L1 into the platform attachment cavity 17266 to a holding position shown in FIG. 16. When release body 17278 is in the holding position, the holding tab 19288 may be longitudinally aligned with the holding opening 18236, which responsively permits the holding prong 19286 to at least partially return to its pre-deflected state (e.g., via the holding prong's natural elastic properties) and the holding tab 19288 to at least partially protrude into the holding opening 18236. The locking tab 19296 is not longitudinally aligned with the locking opening 17238 when in the holding position. The locking prong 19294 thus may be maintained in an elastically deflected state when in the holding position.

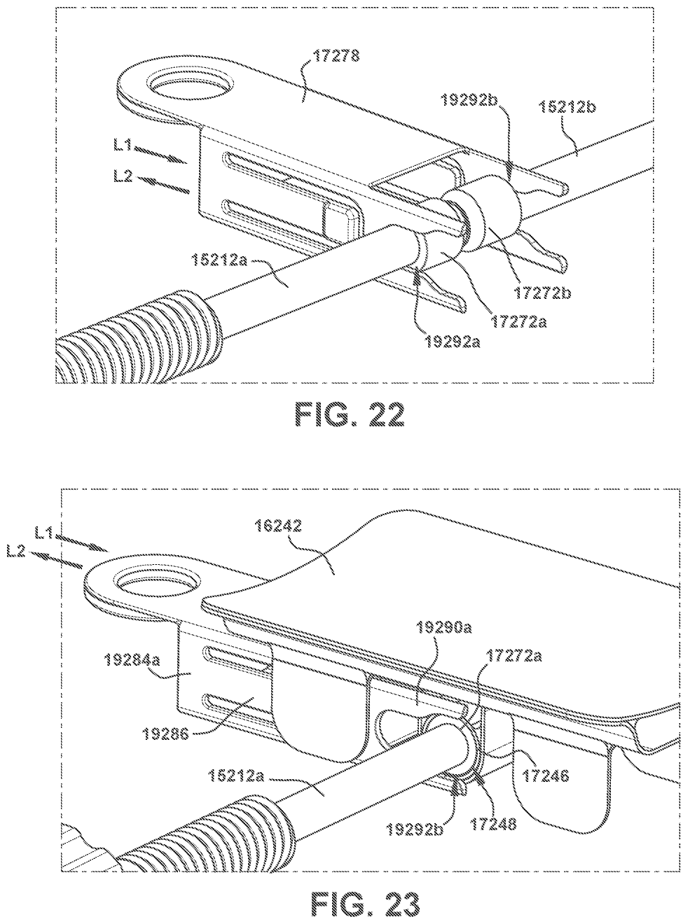

The first claw portions 19292a, 19298a of the first and second release claws 19290a, 19290b are longitudinally aligned with the first and second drive shaft openings 18234a, 1823b when in the holding position. Therefore, a portion of the first drive shaft 15212a extends through the first claw portion 19292a of the first release claw 19290a, and a portion of the second drive shaft 15212b extends through the first claw portion 19298a of the second release claw 19290b. This arrangement can be seen in at least FIGS. 21-22. As shown in FIG. 22, the first and second end supports 17272a, 17272b are larger than the inner transverse heights of the first claw portions 19292a, 19298a. Therefore, when the release body 17278 is in the holding position, the first and second drive shafts 15212a, 15212b are selectively prevented from undesirably egressing from the drive shaft tunnel 17248 through the first and second drive shaft openings 18234a, 18234b and, thus, are selectively prevented from being undesirably detached from the restraint platform 102′. By substantially preventing the detachment of the first and second drive shafts 15212a, 15212b, the release body 17278 also substantially prevents the first and second base housings 16274a, 16274a (and, accordingly, the first and second articulating assemblies 124a′, 124b′ and the first and second sliding members 126a′, 126b′) and the first and second head restraints 122a′, 122a′ from being undesirably detached from the restraint platform 102′. Therefore, the quick release mechanism 16276, when in the holding position, at least partially holds (i.e., retains) components of the head restraint assembly 100′ connected to one another and the head restraint assembly 100′ in the assembled state of FIG. 16.

The quick release mechanism 16276, however, also provides the ability for the medical professional to quickly detach components of the head restraint assembly 100′ from one another when desired. FIGS. 21-22 illustrate portions of the head restraint assembly 100′ in the assembled state and the quick release mechanism 16276 in the holding position. As shown in FIG. 23, when disassembly of the head restraint assembly 100′ is desired, the medical professional may manipulate the release body 17278 to move the release body 17278 from the holding condition to a release condition. For example, the medical professional may grip the gripping portion 19282 of the release body 17278 and urge the release body 17278 in a longitudinal release direction L2 (which is opposite to the longitudinal holding direction L1). As the release body 17278 is urged in the longitudinal release direction, a holding end wall 20302 (FIG. 20) of the holding tab 19288 is urged longitudinally into and engages a portion of the first attachment body sidewall 18230a that defines the holding opening 18236. The holding end wall 20302 is curved and/or angled in such a manner that this engagement causes the holding tab 19288 (and thus the holding prong 19286) to elastically deflect laterally inward so that the holding tab 19288 is removed from the holding opening 18236.

The release body 17278 is moved in the longitudinal release direction until the release body 17278 reaches the release position. In the release position, the locking tab 19296 is longitudinally aligned with the locking opening 17238, which responsively permits the locking prong 19294 to at least partially return to its pre-deflected state (e.g., via the locking tab's natural elastic properties) and the locking tab 19296 to at least partially protrude into the locking opening 17238. The holding tab 19296 is not longitudinally aligned with the holding opening 18236 when in the release position. The holding prong 19286 thus may be maintained in an elastically deflected state when in the release position.

A locking end wall 20304 (FIG. 20) of the locking tab 19296 may be configured to substantially prevent the release body 17278 from being moved further in the longitudinal release direction once the release body 17278 reaches the release position. In particular, unlike the holding end wall 20302, the locking end wall 20304 may extend substantially perpendicular to the second attachment body sidewall 18230b when in the release position. Therefore, if the release body 17278 were to be moved further in the longitudinal release direction, the locking end wall 20304 would be urged into, and engage, a portion of the second attachment body sidewall 18230b that defines the locking opening 17238. Because of the perpendicular configuration, the engagement between the locking end wall 20304 and the second attachment body sidewall 18230b does not cause the locking tab 19296 (and thus the locking prong 19294) to deflect latterly inward. Instead, this engagement substantially prevents further movement of the release body 17278 in the longitudinal release direction. This engagement also at least partially helps prevent the release body 17278 from being undesirably detached from the restraint platform 102′.

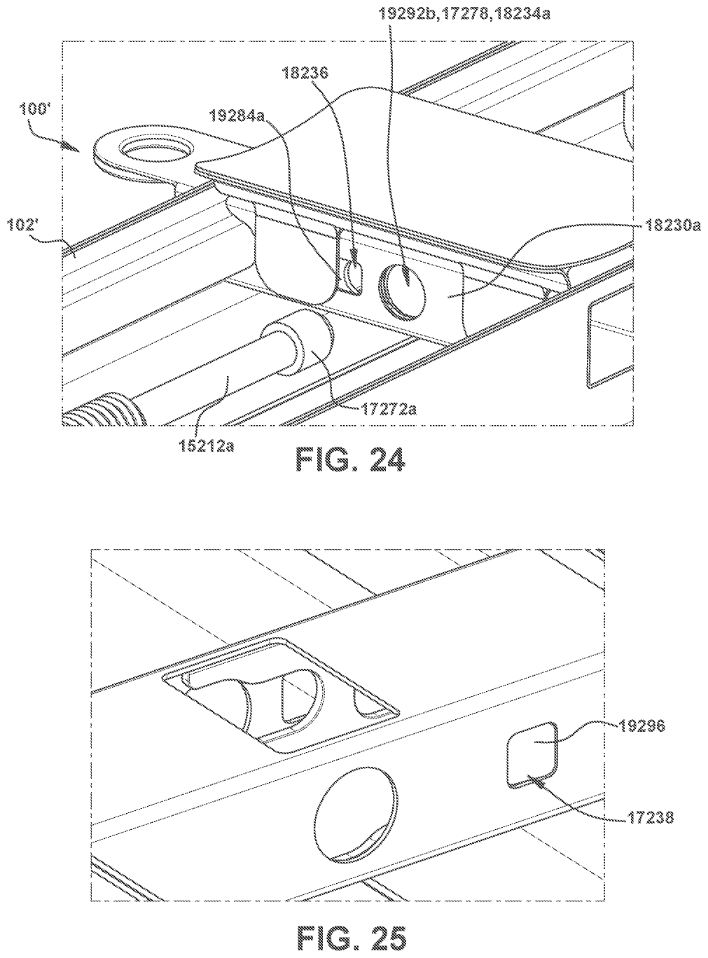

As shown in FIGS. 23-24, the second claw portions 19292b, 19298b of the first and second release claws 19290a, 19290b are longitudinally with the drive shaft tunnel 17248 and the first and second drive shaft openings 18234a, 18234b when in the release position. Therefore, a portion of the first drive shaft 15212a extends through the second claw portion 19292b of the first release claw 19290a, and a portion of the second drive shaft 15212b extends through the second claw portion 19298b of the second release claw 19290b. The first and second end supports 17272a, 17272b are smaller than the inner transverse heights of the second claw portions 19292b, 19298b. Therefore, as shown in FIG. 24, when the release body 17278 is in the release position, the first and second drive shafts 15212a, 15212b may each be selectively removed from the drive shaft tunnel 17248 through the first and second drive shaft openings 18234a, 18234b and, thus, may be detached from the restraint platform 102′. FIG. 25 depicts the release body 17278 in the release position, with the locking tab 19296 at least partially protruding into the locking opening 17238.

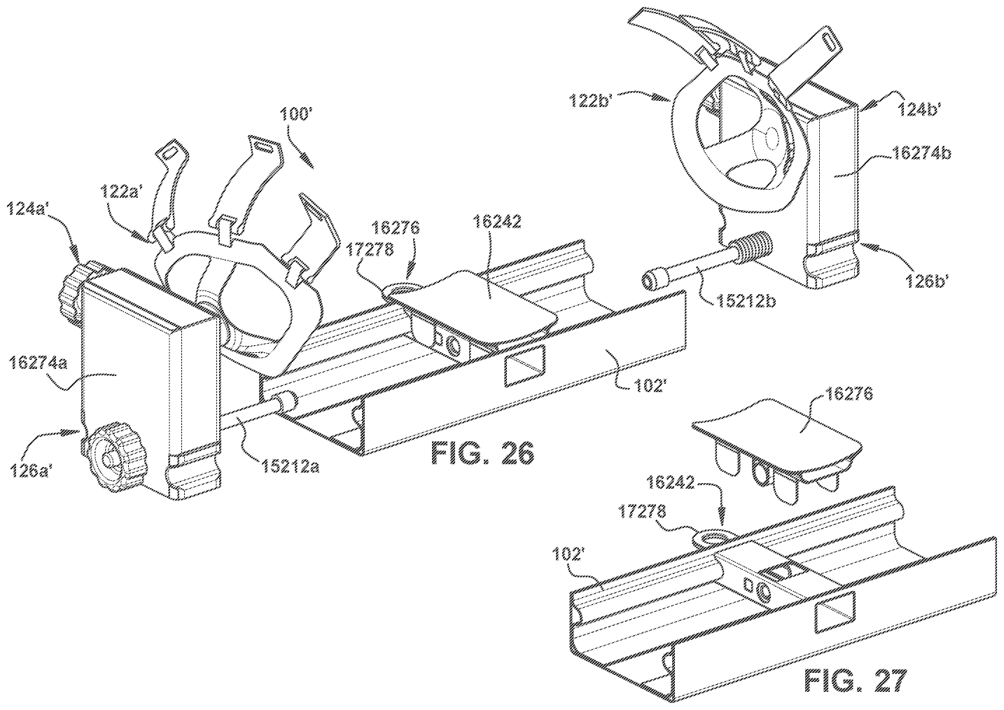

As shown in FIG. 26, the first and second base housings 16274a, 16274b (and, accordingly, the first and second articulating assemblies 124a′, 124b′ and the first and second sliding members 126a′, 126b′) and the first and second head restraints 122a′, 122a′ may be selectively detached from the restraint platform 102′ with the first and second drive shafts 15212a, 15212b. Instead of detaching both, the medical professional may detach only the first head restraint 122a′, the first articulating assembly 124a′, the first sliding member 126a′, and the first base housing 16274a, while leaving the second head restraint 122b′, the second articulating assembly 124b′, the second sliding member 126b′, and the second base housing 16274b attached to the restraint platform 102′, or vice versa.

As shown in FIG. 27, once the drive shafts 15212a, 15212b are removed from the drive shaft tunnel 17248, the head platform 16242 may be selectively removed from the restraint platform 102′. Therefore, the quick release mechanism 16276, when in the release position, at least partially permits components of the head restraint assembly 100′ to be quickly detached from one another and for the head restraint assembly 100′ to be quickly disassembled. The ability for the head restraint assembly 100′ to be quickly disassembled may be beneficial in situations in which the medical professional desires to quickly remove at least the first and second head restraints 122a′, 122b′ from the subject's head H. The disassembly method described above is also beneficial in that the first and second head restraints 122a′, 122b′ may be quickly detached from the restraint platform 102′ and from the subject's head H without having to unfasten any of the straps 370, 490, 4100.

Although not shown in FIGS. 16-17, similarly to the head restraint assembly 100 of FIGS. 1-12 and 15, the head restraint assembly 100′ may include the cheekbone strap 370 that extends between the first and second cheekbone flanges 134a′, 136b′, the forehead strap 490 that extends between the first and second forehead flanges 136a′, 138b′, and the crown strap 4100 that extends between the first and second crown flanges 138a′, 138b′. It is contemplated that the head restraint assembly 100′ may include any desired number of straps that extend from one of the first and second head restraints 122a′, 122b′ to the other of the first and second head restraint 122a′, 122b′.

Furthermore, similar to the head restraint assembly 100 of FIGS. 1-12 and 15, the head restraint assembly 100′ of FIGS. 16-27 is connectable to a head connector 8114. The head connector 8114 may be selectively or substantially permanently connected to at least one of the first platform portion 104a′, the second platform portion 104b′, and the third platform portion 104c′ by one or more fasteners. Each fastener that connects the head connector 8114 to the head restraint assembly 100 may be at least one of an adhesive (such as a permanent adhesive epoxy), tape (such as a high performance acrylic adhesive double-sided tape), a screw (such as a plastic or carbon fiber screw), nail (such as a plastic or carbon fiber nail) a hook and loop fastener, a snap fastener, any suitable metallic or non-metallic fastener, and/or any other suitable fastener or combination of fasteners. It is also contemplated that the head connector 8114 may be integrally formed as a single-piece, unitary construct with at least one of the first platform portion 104a′, the second platform portion 104b′, and the third platform portion 104c′. The head restraint assembly 100′ may also or instead be configured for a press-fit and/or frictional engagement with the head connector 8114 in a manner similar to that of the head restraint assembly 100.

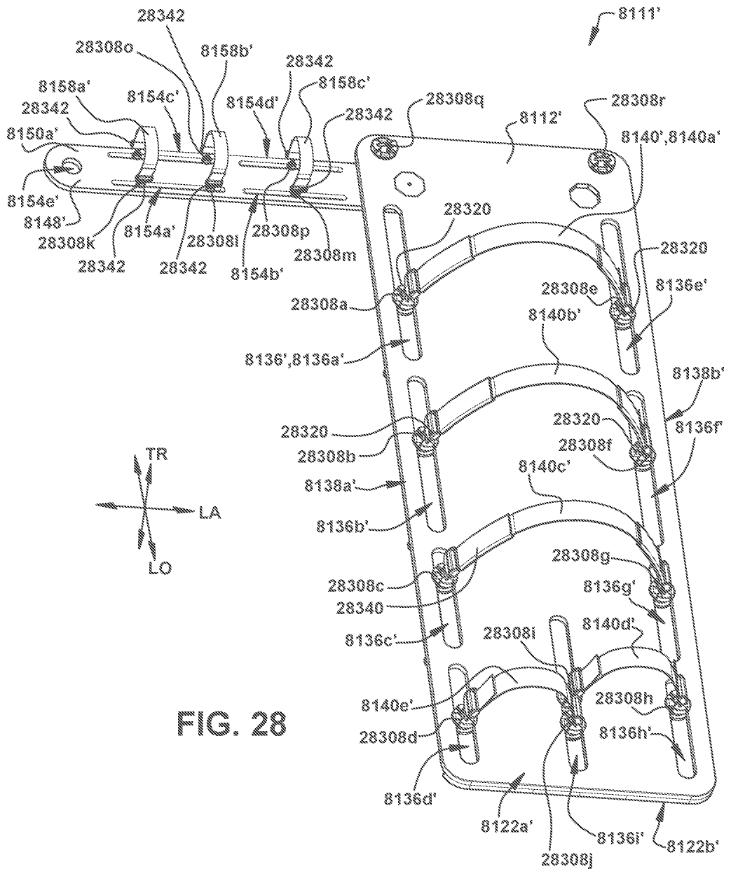

FIGS. 28-36 illustrate a second embodiment of a body stabilizer assembly 8111′ that may be included as part of the restraint assembly 8110. The body stabilizer assembly 8111′ of FIGS. 28-36 is similar to the body stabilizer assembly 8111 of FIGS. 8 and 10-12 and therefore, structures of FIGS. 28-36 that are the same as or similar to those described with reference to FIGS. 8 and 10-12 have the same reference numbers with the addition of a “prime” mark. Description of common elements and operation similar to those in the previously described first embodiment may not be repeated with respect to the second embodiment, but should instead be considered to be incorporated below by reference as appropriate. Furthermore, elements and functionality shown or described with respect to one embodiment may be shared by any of the other embodiments whether or not expressly stated or shown.

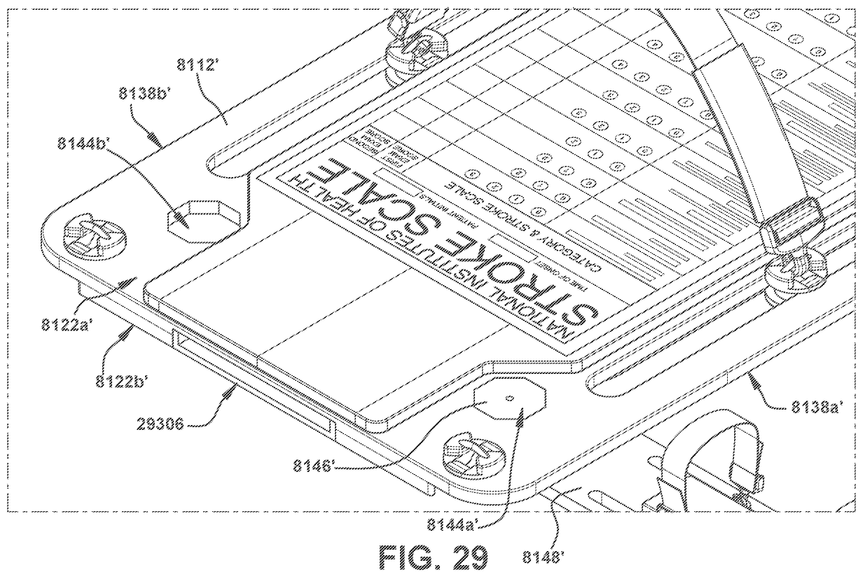

As shown in FIGS. 28-29, the body stabilizer 8112′ has transversely opposing first and second stabilizer surfaces 8122a, 8122b that are separated from one another in the transverse direction. The second stabilizer surface 8122b′ may include any other number of stabilizer bases (e.g., the first and second stabilizer bases 10124a, 10124b) extending transversely therefrom, or any other stabilizing feature(s) extending therefrom for helping to stabilize the body stabilizer when in use.

Via the head connector 8114, either of the head restraint assemblies 100, 100′ may be selected connected to the body stabilizer 8112′. However, instead of including the stabilizer gap 10128 of the body stabilizer 8112 of FIGS. 8 and 10-11, the body stabilizer 8112′ may include a stabilizer pocket 29306 that is configured to selectively receive the second head connector end portion 9120b of the head connector 8114 to join the head connector 8114 to the body stabilizer 8112′ on the first longitudinal stabilizer side 10130a′ of the body stabilizer 8112′. The stabilizer pocket 29306 may be configured so that a press-fit and/or frictional engagement between the body stabilizer 8112′ and the head connector 8114 is created when the head connector 8114 is inserted into the stabilizer pocket 29306. The press-fit and/or frictional engagement between the body stabilizer 8112′ and the head connector 8114 may help prevent the head connector 8114 from being undesirably detached from the body stabilizer 8112′.

Alternatively, or additionally to the press-fit and/or frictional engagement maintaining the head connector 8114 connected to the body stabilizer 8112′, the head connector 8114 may be substantially permanently connected or selectively connected to the body stabilizer 8112′ after being inserted into the stabilizer pocket 29306 via one or more fasteners. Each fastener that connects the head connector 8114 to the body stabilizer 8112′ may be at least one of an adhesive (such as a permanent adhesive epoxy), tape (such as a high performance acrylic adhesive double-sided tape), a screw (such as a plastic or carbon fiber screw), nail (such as a plastic or carbon fiber nail), a hook and loop fastener, a snap fastener, any suitable metallic or non-metallic fastener, and/or any other suitable fastener or combination of fasteners. It is also contemplated that the head connector 8114 may be integrally formed as a single-piece, unitary construct with the body stabilizer 8112′.

As shown in FIG. 28, the body stabilizer 8112′ may include at least one stabilizer aperture 8136′ (shown here as stabilizer apertures 8136a′-i′) extending transversely therethrough. In the example configuration of FIG. 28, the body stabilizer 8112′ includes first through fourth stabilizer apertures 8136a′-d′ longitudinally positioned along the first lateral stabilizer side 8138a′ of the body stabilizer 8112′. The body stabilizer 8112′ also includes fifth through eighth stabilizer apertures 8136e′-h′ longitudinally positioned along the second lateral stabilizer side 8138b′ of the body stabilizer 8112′. The first through fourth stabilizer apertures 8136a′-d′ may be longitudinally aligned or longitudinally offset from the fifth through eighth stabilizer apertures 8136e′-h′. A ninth stabilizer aperture 8136i′ may be positioned adjacent the second longitudinal stabilizer side 10130b′ of the body stabilizer 8112′ and laterally between the stabilizer apertures 8136a′-d′ on the first lateral stabilizer side 8138a′ and the stabilizer apertures 8136e′-h′ on the second lateral stabilizer side 8138b′. Any of the stabilizer apertures 8136a′-i′ may be used by at least one medical professional to grip the body stabilizer 8112′, such as to transport a restrained subject S.

As shown in FIG. 28, the body stabilizer assembly 8111′ may include at least one restraining strap 8140′ (shown here as restraining straps 8140a′-e′) configured to be selectively attached to the body stabilizer 8112′ and selectively prevent the egress of the subject S from the body stabilizer 8112′. The at least one restraining strap 8140′ may also be configured to immobilize at least a portion of the subject's body B on the body stabilizer 8112′. The body stabilizer assembly 8111′ may include, for example, first through fifth restraining straps 8140a′-e′.

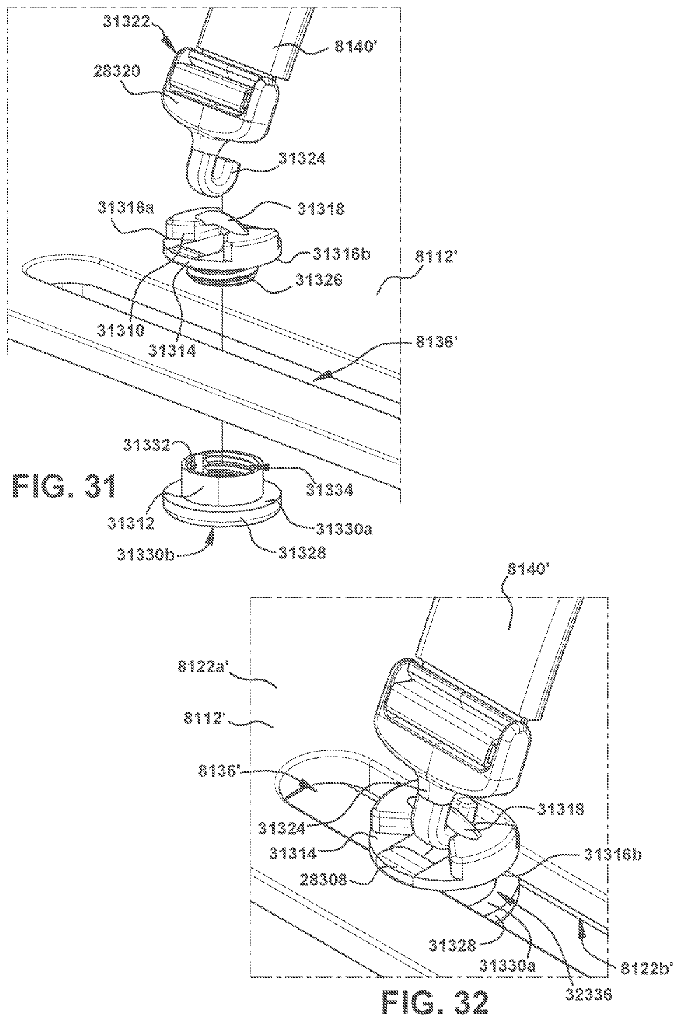

As shown in FIG. 28, each restraining strap 8140′ may be selectively attached to the body stabilizer 8112′ via at least one strap connector 28308 (shown here as first through tenth strap connectors 28308a-j). As shown in FIGS. 31-32, each strap connector 28308 includes first and second strap connector portions 31310, 31312. The first connector portion 31310 includes a first connector flange 31314. A first surface 31316a of the first connector flange 31314 includes a connector attachment portion 31318 to which an end of an associated restraining strap 8140′ may be selectively connected. For example, as shown in FIGS. 28 and 31-32, each end of each restraining strap 8140′ may have a restraining strap attachment member 28320 connected thereto. As shown in FIGS. 31-32, each restraining strap attachment member 28320 may include a buckle portion 31322 through which an associated end of an associated restraining strap 8140′ is looped and connected to the restraining strap attachment member 28320. Although the ends of the restraining straps 8140′ are described as being joined to the restraining strap attachment members 28320 via a buckle-type connection, the restraining straps 8140′ may be joined to the restraining strap attachment members 28320 in any other desired manner.

Each restraining strap attachment member 28320 may also include an attachment portion 31324 through which the restraining strap attachment member 28320 and the associated end of an associated restraining strap 8140′ is connected to the connector attachment portion 31318. In the configuration shown in FIGS. 31-32, the attachment portion 31324 of the restraining strap attachment member 28320 is in the form of a hook, while the connector attachment portion 31318 is in the form of a bar to which the hook may be selectively attached. However, the restraining strap attachment member 28320 may be connected to the first strap connector portion 31310 in any other desired manner, such as, for example, via the use of at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

A second surface 31316b of the first connector flange 31314 may include a threaded body 31326 extending transversely therefrom for selectively attaching the first strap connector portion 31310 to the second strap connector portion 31312. The second strap connector portion 31312 includes a second connector flange 31328 having transversely opposite first and second surfaces 31330a, 31330b. The first surface 31330a of the second connector flange 31328 includes a receiving body 31332 extending transversely therefrom. The receiving body 31332, although being depicted as being a cylindrical receiving body 31332, may have any desired shape. The receiving body 31332 includes internal threads 31334 via which the threaded body 31326 may selectively threadably engage to connect the first and second connector portions 31310, 31312 together. Although the first and second connector portions 31310, 31312 are described and depicted as being connected together via a threaded engagement, the first and second connector portions 31310, 31312 may be configured to be selectively directly or indirectly connected in any other desired manner.

As shown in FIG. 32, when the strap connector 28308 is connected to the body stabilizer 8112′, the second surface 31316b of the first connector flange 31314 engages and/or is adjacent to the first stabilizer surface 8122a′, the first surface 31330a of the second connector flange 31328 engages and/or is adjacent to the second stabilizer surface 8122b′, and the receiving body 31332 is positioned in an associated stabilizer aperture 8136′. The second surface 31316b of the first connector flange 31314, the first surface 31330a of the second connector flange 31328, and the receiving body 31332 thus define a groove 32336 (e.g., an annular groove) that is configured to receive portions of the body stabilizer 8112′ adjacent the associated stabilizer aperture 8136′. When the strap connectors 28308 are attached to the body stabilizer 8112′ in the manner described above, each end of each restraining strap 8140′ may be selectively connected to the body stabilizer 8112′ by connecting the restraining strap attachment members 28320 to the strap connectors 28308 in the manner described above.

Each of the strap connectors 28308 may be selectively longitudinally movable along their respective stabilizer apertures 8136a′-i′ relative to the body stabilizer 8112′. This longitudinal movement allows each of the first through fifth restraining straps 8140a′-e′ to be placed in selected longitudinal positions along the subject's body B in order to correspond to the subject's anatomy. The strap connectors 28308 may be freely movable (i.e., moveable when a small or insignificant amount of force is applied thereto) along their respective stabilizer apertures 8136a′-i′, or may be movable only when a significant predetermined amount of force is applied thereto. The strap connectors 28308 may also or instead be configured such that when the first and second connector portions 31310, 31312 are fully connected to one another, the first and second connector portions 31310, 31312 apply a compressive force to the body stabilizer 8112′ that substantially prevents the strap connectors 28308 from being undesirably moved along their respective stabilizer apertures 8136a′-i′. In such a configuration, when movement of a selected strap connector 28308 is desired, the medical provisional may at least partially disengage the first and second connector portions 31310, 31312 from one another to alleviate at least a portion of the compressive force, move the strap connector 28308 to a desired longitudinal position, and then fully rejoin the first and second connector portions 31310, 31312 to one another.

Although the first and second connector portions 31310, 31312 have been described as being connected together via a threaded engagement, any other engagement may be provided, such as, but not limited to, a magnetic engagement, hook and loop engagement, a nail and nail hole engagement, a snap-fit engagement, an adhesive engagement, a screw and nut engagement, a split pin engagement, a button engagement, a press-fit engagement, any other suitable engagement, or any combination thereof. Further, although the strap connectors 28308 have been described as having two separate subcomponents (i.e., the first and second connector portions 31310, 31312), each strap connector 28308 may be formed as a single-piece, unitary construct, or have any desired number of subcomponents.

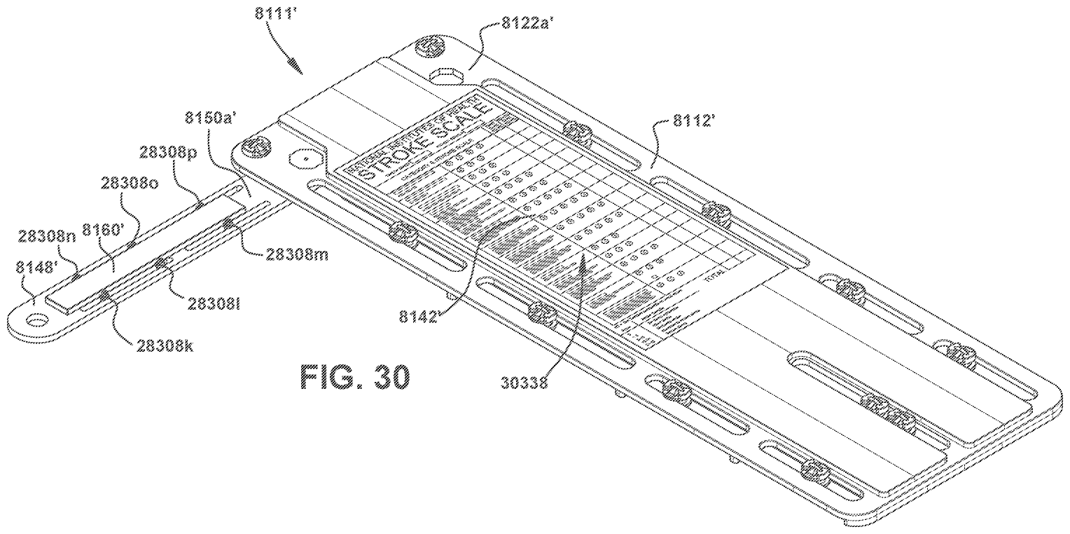

As shown in FIG. 30, the first stabilizer surface 8122a′ may have a reusable or disposable stabilizer cushion 8142′ provided thereon that is configured to be interposed between the first stabilizer surface 8122a′ and the subject's body B, legs L, and/or feet FT. The stabilizer cushion 8142′ provides a softer surface for the subject to be laid on than the substantially rigid first stabilizer surface 8122a′. The stabilizer cushion 8142′ may be substantially permanently attached to the first stabilizer surface 8122a′ or may be configured to be selectively removed from the first stabilizer surface 8122a′ as desired. The stabilizer cushion 8142′ may also be configured to have medical information 30338 (e.g., the National Institutes of Health's Stroke Scale) provided (e.g., printed) thereon for the medical professional to reference as needed. As shown in FIG. 28, at least one of the restraining straps 8140′ includes a padded strap cover 28340 connected thereto configured to be interposed between the restraining strap(s) 8140′ and the subject's body B, legs L, and/or feet FT.

As shown in FIGS. 28-29 and 33, at least one of the first and second lateral stabilizer sides 8138a′, 8138b′ may include a winglet attachment aperture 8144a′, 8144b′ extending transversely therethrough. For example, the body stabilizer 8112′ shown in FIGS. 28-29 and 33 includes a first winglet attachment aperture 8144a′ on the first lateral stabilizer side 8138a′ and a second winglet attachment aperture 8144b′ on the second lateral stabilizer side 8138b′. Each of the first and second winglet attachment apertures 8144a′, 8144b′ is configured to selectively receive a winglet attachment projection 8146′ of a winglet 8148′ to attach the winglet 8148′ to the body stabilizer 8112′.

The winglet 8148′ is configured to selectively receive an arm A of the subject S. The winglet attachment projection 8146′ extends transversely from the first winglet surface 8150a′ on the first lateral winglet side 8152a′ of the winglet 8148′. When the winglet 814′8 is attached to the body stabilizer 8112′, the first winglet surface 8150a′ abuts or is adjacent to the second stabilizer surface 8122b′.

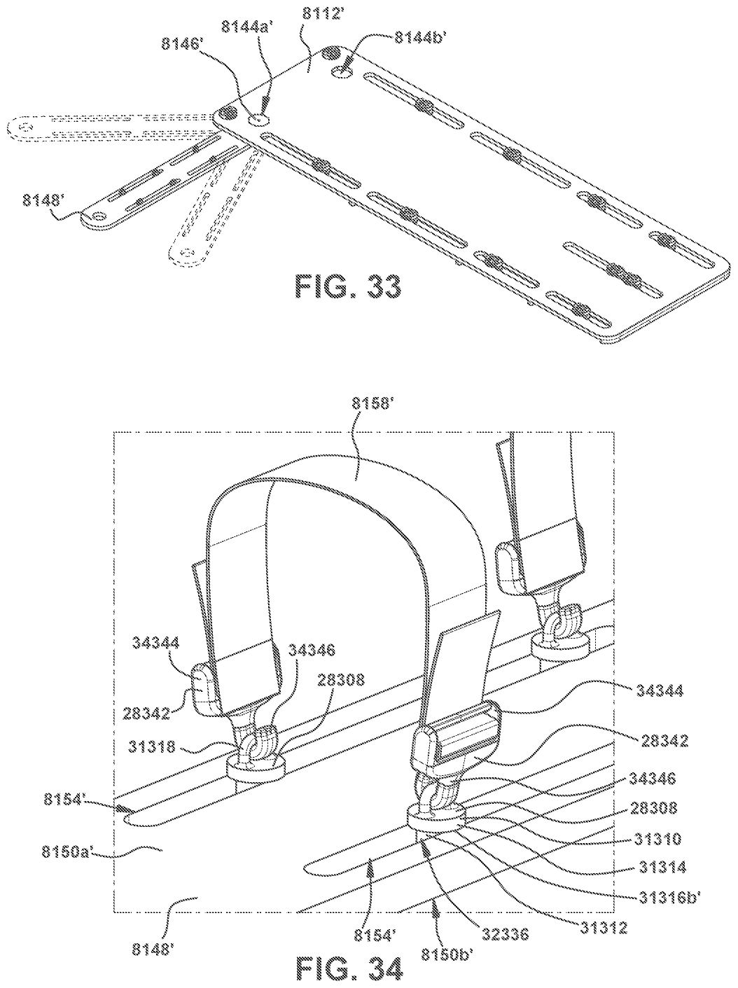

The winglet attachment projection 8146′ and the first and second winglet attachment apertures 8144a′, 8144b′ are configured such that the winglet 8148′ may be selectively pivoted relative to the body stabilizer 8112′ to a desired angular position for receiving the subject's arm A. Pivoting the winglet 8148′ to a desired angular position may include disconnecting the winglet 8148′ from the body stabilizer 8112′ by removing the winglet attachment projection 8146′ from one of the first and second winglet attachment apertures 8144a′, 8144b′, pivoting the winglet 8148′ relative to the body stabilizer 8112′ to the desired angular position, and then reattaching the winglet 8148′ to the body stabilizer with the winglet 8148′ in the desired angular position. The winglet attachment projection 8146′ and the first and second winglet attachment apertures 8144a′, 8144b′ may be keyed or indexed such that the winglet 8148′ is pivotable at predetermined angular increments relative to the body stabilizer 8112′ (as shown in the configuration of FIGS. 28 and 33), or the winglet 8148′ may be freely pivotable relative to the body stabilizer 8112′. The keying or indexing may comprise substantially shaping at least a portion of the winglet attachment projection 8146′ and the first and second winglet attachment apertures 8144a′, 8144b′ as an octagon, any other polygon, or any other desired shape.

As shown in FIG. 28, the winglet 8148′ may include at least one winglet aperture 8154′ (shown here as winglet apertures 8154a′-e′) extending transversely therethrough. In the example configuration and orientation of FIG. 28, the winglet 8148′ includes first and second winglet apertures 8154a′, 8154b′ laterally positioned along the first longitudinal winglet side 8156a′ of the winglet 8148′. The winglet 8148′ also includes third and fourth winglet apertures 8154c′, 8154d′ laterally positioned along the second longitudinal winglet side 8156b′ of the winglet 8148′. The first and second winglet apertures 8154a′, 8154b′ may be laterally aligned or laterally offset from the third and fourth winglet apertures 8154c′, 8154d′. A fifth winglet aperture 8154e′ may extend transversely through the winglet 8148′ on the second lateral winglet side 8152b′ of the winglet 8148′. Any of the winglet apertures 8154a′-e′ may be used by at least one medical professional to grip the winglet 8148′ when transporting a restrained subject S and/or to manipulate the subject's arm A.

As shown in FIGS. 28, the body stabilizer assembly 8111′ may include at least one winglet strap 8158′ (shown here as winglet straps 8158a′-c′) configured to be selectively attached to the winglet 8148′ and selectively prevent the egress of the subject's arm A from the winglet 8148′. The at least one winglet strap 8158′ may also be configured to immobilize at least a portion of the subject's arm A on the winglet 8148′. The restraint assembly 8110 may include, for example, first through third winglet straps 8158a′-c′.

Each winglet strap 8158′ may be selectively attached to the winglet 8148′ via at least one strap connector 28308 (shown in FIGS. 28 and 30 as eleventh through sixteenth strap connectors 28308k-p). The strap connectors 28308 for the winglet straps 8158′ are substantially the same as the strap connectors 28308 for the restraining straps 8140a′-e′, though the strap connectors 28308 for the winglet straps 8158′ may be smaller. Therefore, the above structural descriptions of the strap connectors 28308 for the restraining straps 8140a′-e′ may also be considered as applicable to the strap connectors 28308 for the winglet straps 8158′.

As shown in FIGS. 28 and 34, each end of each winglet strap 8158′ may have a winglet strap attachment member 28342 connected thereto. As shown in FIG. 34, each winglet strap attachment member 28342 may include a buckle portion 34344 through which an associated end of an associated winglet strap 8158′ is looped and connected to the winglet strap attachment member 28342. Although the ends of the winglet straps 8158′ are described as being joined to the winglet strap attachment members 28342 via a buckle-type connection, the winglet straps 8158′ may be joined to the winglet strap attachment members 28342 in any other desired manner.

Each winglet strap attachment member 28342 may also include an attachment portion 34346 through which the winglet strap attachment member 28342 and the associated end of an associated winglet strap 8158′ is connected to the connector attachment portion 31318. In the configuration shown in FIG. 34, the attachment portion 34346 of the winglet strap attachment member 28342 is in the form of a hook, while the connector attachment portion 31318 is in the form of a bar to which the hook may be selectively attached. However, the winglet strap attachment member 28342 may be connected to the first strap connector portion 31310 in any other desired manner, such as, for example, via the use of at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

As shown in FIG. 34, when the strap connector 28308 is connected to the winglet 8148′, the second surface 31316b of the first connector flange 31314 engages and/or is adjacent to the first winglet surface 8150a′, the first surface 31330a of the second connector flange 31328 engages and/or is adjacent to the second winglet surface 8150b′, and the receiving body 31332 is positioned in an associated winglet aperture 8154′. The second surface 31316b of the first connector flange 31314, the first surface 31330a of the second connector flange 31328, and the receiving body 31332 thus define a groove 32336 (e.g., an annular groove) that is configured to receive portions of the winglet 8148′ adjacent the associated winglet aperture 8154′. When the strap connectors 28308 are attached to the winglet 8148′ in the manner described above, each end of each winglet strap 8158′ may be selectively connected to the winglet 8148′ by connecting the winglet strap attachment members 28342 to the strap connectors 28308 in the manner described above.

Each of the strap connectors 28308 may be selectively longitudinally movable along their respective winglet apertures 8154a′-d′. This longitudinal movement allows each of the first through third winglet straps 8158a′-c′ to be placed in selected longitudinal positions along the subject's arm A in order to correspond to the subject's anatomy. The strap connectors 28308 may be freely movable (i.e., moveable when a small or insignificant amount of force is applied thereto) along their respective winglet apertures 8154a′-d′, or may be movable only when a significant predetermined amount of force is applied thereto. The strap connectors 28308 may also or instead be configured such that when the first and second connector portions 31310, 31312 are fully connected to one another, the first and second connector portions 31310, 31312 apply a compressive force to the winglet 8148′ that substantially prevents the strap connectors 28308 from being undesirably moved along their respective winglet apertures 8154a′-d′. In such a configuration, when movement of a selected strap connector 28308 is desired, the medical professional may at least partially disengage the first and second connector portions 31310, 31312 from one another to alleviate at least a portion of the compressive force, move the strap connector 28308 to a desired longitudinal position, and then fully rejoin the first and second connector portions 31310, 31312 to one another.

As shown in FIG. 30, the first winglet surface 8150a′ may have a reusable or disposable winglet cushion 8160′ provided thereon that is configured to be interposed between the subject's arm A and the first winglet surface 8150a′. The winglet cushion 8160′ provides a softer surface for the subject's arm A to be laid on than the substantially rigid first winglet surface 8150a′. The winglet cushion 8160′ may be substantially permanently attached to the first winglet surface 8150a′ or may be configured to be selectively removed from the first winglet surface 8150a′ as desired.

Although the body stabilizer assembly 8111′ is shown as having only one winglet 8148′, the body stabilizer assembly 8111′ may have any number of winglets 8148′. For example, one winglet 8148′ may be joined to the first winglet attachment aperture 8144a′, while a second winglet 8148′ may be attached to the second winglet attachment aperture 8144b′.

Unlike the body stabilizer assembly 8111 of FIGS. 8 and 10-11, the body stabilizer assembly 8111′ of FIGS. 28-34, may be provided without a separate foot platform 8162, though it is contemplated that the body stabilizer assembly 8111′ may include a separate foot platform 8162 as desired. When the separate foot platform 8162 is omitted, the lateral length of the body stabilizer 8112′ may be configured to receive a portion of the subject's feet FT and/or a portion of the subject's legs L. The fourth and fifth restraining straps 8140d′, 8140e′ may be configured to selectively prevent the egress of the subject's feet FT and/or legs L from the body stabilizer 8112′. The fourth and fifth restraining straps 8140d′, 8140e′ may also be configured to immobilize at least a portion of the subject's feet FT and/or legs L on the body stabilizer 8112′. Based on its positioning, the third restraining strap 8140c′ may also be configured to selectively prevent the egress of the subject's legs L from the body stabilizer 8112′, as well as immobilize at least a portion of the subject's legs L on the body stabilizer 8112′

It is also contemplated that the body stabilizer 8112′ may be configured to receive at least a portion of the subject's head H. In such case, the body stabilizer assembly 8111′ may be used without the head restraint assembly 100, 100′. In another non-head restraint assembly configuration, the body stabilizer assembly 8111′ may include the head connector 8114 selectively or permanently joined to the body stabilizer 8112′. In such a configuration, the head connector 8114 may be configured to receive the subject's head H, while the body stabilizer 8112′ receives at least one of the subject's body B, arm(s) A, leg(s) L, and foot/feet FT.

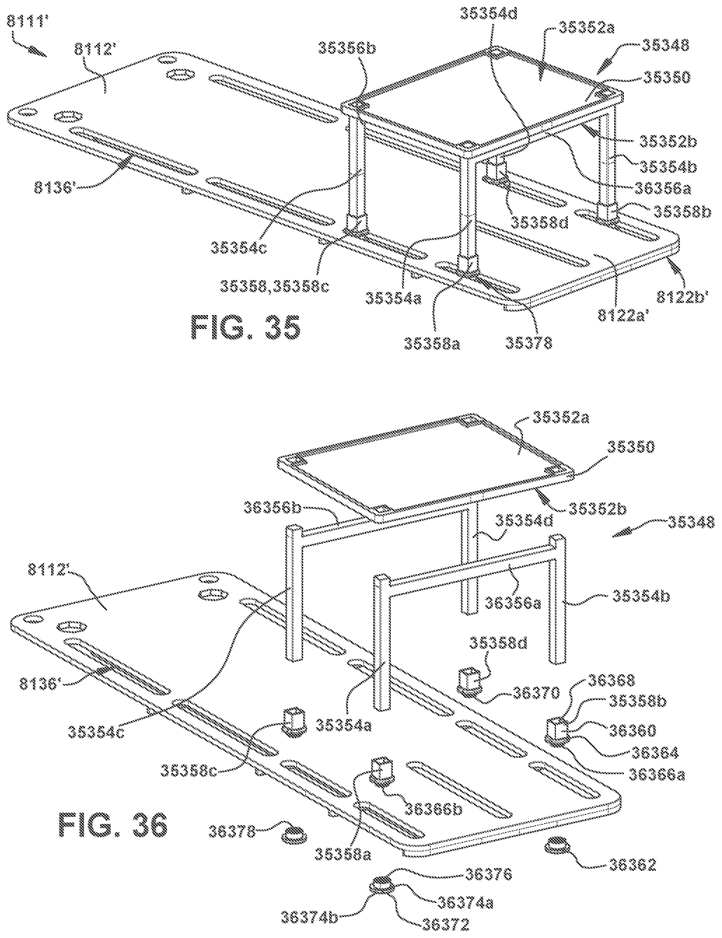

As shown in FIGS. 35-36, the body stabilizer assembly 8111′ may include an attachable tray assembly 35348. The tray assembly 35348 includes a tray table 35350 having transversely opposite first and second tray table surfaces 35352a, 35352b. In use the first tray table surface 35352a may face away from the body stabilizer 8112′ and be configured to hold medical instruments, medical supplies, any other desired object, or any combination thereof. The first tray table surface 35352a thus may be utilized as a surgical tray.

The second tray table surface 35352b may face toward the body stabilizer 8112′ during use. The second tray table surface 35352b is selectively connected to at least one tray leg 35354 (shown here as tray legs 35354a-d). In the example configuration of FIGS. 35-36, the second tray table surface 35352b is connectable to first through fourth tray legs 35354a-d. At least one of the tray legs 35354 may be integrally joined to another one of the tray legs 35354 via a leg connector 36356 (shown here as leg connectors 36356a and 36356b). In the example configuration of FIGS. 35-36, a first leg connector 36356a extends between and connects the first tray leg 35354a to the second tray leg 35354b, while a second leg connector 36356b extends between and connects the third tray leg 35354c to the fourth tray leg 35354d.

The tray assembly 35348 may include at least one tray connector 35358 (shown here as first through fourth tray connectors 35358a-d) for selectively connecting the at least one tray leg 35354 to the body stabilizer 8112′. In particular, the tray assembly 35348 may have one tray connector 35358 for each tray leg 35354. Therefore, in the example configuration of FIGS. 35-36, the tray assembly 35348 includes first through fourth tray connectors 35358a-d.

As shown in FIG. 36, each tray connector 35358 includes first and second tray connector portions 36360, 36362. The first tray connector portion 36360 includes a first tray connector flange 36364. A first surface 36366a of the first tray connector flange 36364 includes a first receiving body 36368 extending transversely therefrom for selectively receiving an end of an associated tray leg 35354. The first receiving body 36368 and its associated tray leg 35354 may be configured so that a press-fit and/or frictional engagement is created between the first receiving body 36368 and the associated tray leg 35354 when the associated tray leg 35354 is inserted into the first receiving body 36368.

Although the first receiving bodies 36368 and the tray legs 35354 have been described as being connected together via a press-fit and/or frictional engagement, any other engagement may be provided, such as, but not limited to, a magnetic engagement, hook and loop engagement, a nail and nail hole engagement, a snap-fit engagement, an adhesive engagement, a screw and nut engagement, a split pin engagement, a button engagement, a threaded engagement, any other suitable engagement, or any combination thereof.

A second surface 36366b of the first tray connector flange 36364 may include a threaded body 36370 extending transversely therefrom for selectively attaching the first tray connector portion 36360 to the second tray connector portion 36362. The second tray connector portion 36362 includes a second tray connector flange 36372 having transversely opposite first and second surfaces 36374a, 36374b. The first surface 36374a of the second tray connector flange 36372 includes a second receiving body 36376 extending transversely therefrom. The second receiving body 36376, although depicted in the Figures as being a cylindrical receiving body 36376, may have any desired shape. The second receiving body 36376 includes internal threads 36378 which the threaded body 36370 may selectively threadably engage to connect the first and second tray connector portions 36360, 36362 together. Although the first and second tray connector portions 36360, 36362 are described and depicted as being connected together via a threaded engagement, the first and second tray connector portions 36360, 36362 may be configured to be joined together in any other desired manner.

As shown in FIG. 35, when each tray connector 35358 is connected to the body stabilizer 8112′, the second surface 36366b of the first tray connector flange 36364 engages and/or is adjacent to the first stabilizer surface 8122a′, the first surface 36374a of the second tray connector flange 36372 engages and/or is adjacent to the second stabilizer surface 8122b′, and the second receiving body 36376 is positioned in an associated stabilizer aperture 8136′. The second surface 36366b of the first tray connector flange 36364, the first surface 36374a of the second tray connector flange 36372, and the second receiving body 36376 thus define a groove 35380 (e.g., an annular groove) that is configured to receive portions of the body stabilizer 8112′ adjacent the associated stabilizer aperture 8136′. When the tray connectors 35358 are attached to the body stabilizer 8112′ in the manner described above, the tray legs 35354 may be selectively connected to the body stabilizer 8112′ by joining the tray legs 35354 to the tray connectors 35358 in the manner described above.

Although the first and second tray connector portions 36360, 36362 have been described as being connected together via a threaded engagement, any other engagement may be provided, such as, but not limited to, a magnetic engagement, hook and loop engagement, a nail and nail hole engagement, a snap-fit engagement, an adhesive engagement, a screw and nut engagement, a split pin engagement, a button engagement, a press-fit engagement, any other suitable engagement, or any combination thereof. Further, although the tray connectors 35358 have been described as having two separate subcomponents (i.e., the first and second tray connector portions 36360, 36362), each tray connector 35358 may be formed as single-piece, unitary construct or have any desired number of subcomponents.

Each of the tray connectors 35358 may be selectively longitudinally movable along a respective stabilizer apertures 8136′ relative to the body stabilizer 8112′. This longitudinal movement allows the tray assembly to be placed in selected longitudinal positions along the body stabilizer 8112′. The tray connectors 35358 may be freely movable (i.e., moveable when a small or insignificant amount of force is applied thereto) along respective stabilizer apertures 8136′, or may be movable only when a significant predetermined amount of force is applied thereto. The tray connectors 35358 may also or instead be configured such that when the first and second tray connector portions 36360, 36362 are fully connected to one another, the first and second tray connector portions 36360, 36362 apply a compressive force to the body stabilizer 8112′ that substantially prevents the tray connectors 35358 from being undesirably moved along their respective stabilizer apertures 8136′. In such a configuration, when movement of a selected tray connector 35358 is desired, the medical provisional may at least partially disengage the first and second tray connector portions 36360, 36362 from one another to alleviate at least a portion of the compressive force, move the tray connector 35358 to a desired longitudinal position, and then at least partially rejoin the first and second tray connector portions 36360, 36362 together. Furthermore, because the tray assembly 35348 is comprised of a plurality of detachable parts (i.e., the tray table 35350, the tray legs 35354, and the tray connectors 35358), the tray assembly 35348 may be easily attached and detached from the body stabilizer 8112′ as desired.

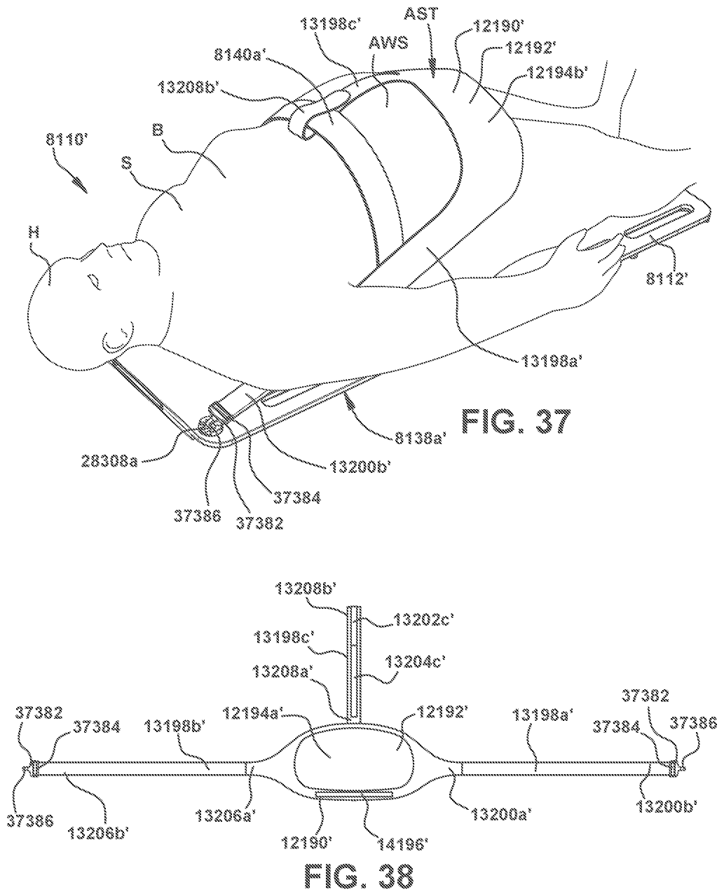

FIGS. 37-38 illustrate a second embodiment of abdominal sling 12190′ that may be included as part of the restraint assembly 8110. The abdominal sling 12190′ of FIGS. 37-38 is similar to the abdominal sling 12190 of FIGS. 12-14 and therefore, structures of FIGS. 37-38 that are the same as or similar to those described with reference to FIGS. 12-14 have the same reference numbers with the addition of a “prime” mark. Description of common elements and operation similar to those in the previously described first embodiment may not be repeated with respect to the second embodiment, but should instead be considered to be incorporated below by reference as appropriate. Furthermore, elements and functionality shown or described with respect to one embodiment may be shared by any of the other embodiments whether expressly stated, shown, or not.

As shown in FIGS. 37-38, the abdominal sling 12190′ includes a central sling portion 12192′ having a first sling surface 12194a′ configured to selectively engage the subject's abdominal wall skin AWS, and a second sling surface 12194b′ configured to face away from the subject's abdominal wall skin AWS when the first sling surface 12194a′ is engaged to the subject S. The first sling surface 12194a′ may include a biocompatible adhesive 14196′ provided thereon for selectively maintaining the first sling surface's engagement to the subject's abdominal wall skin AWS. Alternatively or additionally, the first sling surface 12194a′ may include a biocompatible gel pad or other friction-increasing member provided thereon for selectively maintaining the first sling surface's engagement to the subject's abdominal wall skin AWS

The abdominal sling 12190′ also has at least one sling strap 13198′ (shown here as sling straps 13198a′, 13198b′, 13198c′) extending from the central sling portion 12192′. In the example configuration shown in FIGS. 37-38, the abdominal sling includes the first, second, and third sling straps 13198a′, 13198b′, 13198c′ extending from the central sling portion 12192′. The second ends 13200b′, 13206b′ of the first and second sling straps 13198a′, 13198b′ may each include a sling strap attachment member 37382 connected thereto. The sling strap attachment members 37382 are substantially the same as the restraining strap attachment members 28234. Therefore, each sling strap attachment member 37382 includes a buckle portion 37384 through which an associated second end 13200b′, 13206b′ of an associated sling strap 13198a′, 13198b′ is looped and connected to the sling strap attachment member 37382. Although the ends of the first and second sling straps 13198a′, 13198b′ are described as being joined to the sling strap attachment members 37382 via a buckle-type connection, the first and second sling straps 13198a′, 13198b′ may be joined to the sling strap attachment members 37382 in any other desired manner such as, but not limited to, a snap-type connection, a ring-type connection, a sewn or adhered connection, and/or a toggle-type connection.

As shown in FIGS. 28 and 37, the body stabilizer 8112′ may include strap connectors 28308 (shown here as seventeenth and eighteenth strap connectors 28308q-r) for the first and second sling straps 13198a′, 13198b′. The strap connectors 28308 for the sling straps 13198a′, 13198b′ are substantially the same as the strap connectors 28308 for the restraining straps 8140a′-e′, though the strap connectors 28308 for the sling straps 13198a′, 13198b′ may be a different size. Therefore, the above structural descriptions of the strap connectors 28308 for the restraining straps 8140a′-e′ may also be considered as applicable to the strap connectors 28308 for the sling straps 13198a′, 13198b′. Each strap connector 28308 for the sling straps 13198a′, 13198b′ extends through an associated stabilizer aperture 8136′ (shown here as tenth and eleventh stabilizer apertures 8136j′-k′).

Each sling strap attachment member 37382 may also include an attachment portion 37386 through which the sling strap attachment member 37382 and the associated second end 13200b′, 13206b′ of an associated sling strap 13198a′, 13198b′ is connected to an associated connector attachment portion 31318 of an associated strap connector 28308q-r. In the configuration shown in FIGS. 37-38, the attachment portion 37386 of the sling strap attachment member 37382 is in the form of a hook, while the connector attachment portion 31318 is in the form of a bar to which the hook may be selectively attached. However, the sling strap attachment member 37382 may be connected to the first strap connector portion 31310 in any other desired manner, such as, for example, via the use of at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners.

As shown in FIGS. 37-38, the third sling strap 13198c′ positioned laterally between the first and second sling straps 13198a′, 13198b′. Therefore, a second end 13208b′ of the third sling strap 13198c′ may be selectively looped around a selected restraining strap 8140′ between the first and second lateral stabilizer sides 8138a′, 8138b′ and pulled toward the central sling portion 12192′ to tension the third sling strap 13198c′. Once the third sling strap 13198c′ is tensioned as desired, the second end 13208b′ of the third sling strap 13198c′ may be selectively attached to another portion of the third sling strap 13198c′ to maintain the desired tensioning in the third sling strap 13198c′. The second end 13208b′ of the third sling strap 13198c′ may be attached to the other portion of the third sling strap 13198c′ via at least one of a clip, an adhesive, a hook and loop fastener, a magnetic fastener, a snap fastener, a hook and eye fastener, a button, a string and eye fastener, a saw tooth fastener, any suitable metallic or non-metallic fastener, and/or any other suitable fastener or combination of fasteners. As shown in FIG. 38, the second end 13208b′ of the third sling strap 13198c′ may include a hook portion 13202c′ that is configured to be connected to a loop portion 13204c′ on the third sling strap 13198c′ to attach the second end 13208b′ of the third sling strap 13198c′ to the remainder of the third sling strap 13198c′.

When the central sling portion 12192′ is engaged to the subject's abdominal wall skin AWS, the first sling strap 13198a′ may be pulled toward and connected to the seventeenth strap connector 28308q, and the second sling strap 13198b′ may be pulled toward and connected to the eighteenth strap connector 28308r. Through these connections, the first sling strap 13198a′ may be connected to the first lateral stabilizer side 8138a′ of the body stabilizer 8112′, while the second sling strap 13198b′ may be connected to the second lateral stabilizer side 8138b′ of the body stabilizer 8112′. The pulling of the first and second sling straps 13198a′, 13198b′ may tension the first and second sling straps 13198a′, 13198b′. The tensioning of the first and second sling straps 13198a′, 13198b′ may be adjusted at the buckle portions 37384 of the sling strap attachment members 37382. The third sling strap 13198c′ may also be selectively tensioned and connected to a selected restraining strap 8140′ in the manner described above.

The tensioning in at least one of the first, second, and third sling straps 13198a′, 13198b′, 13198c′ is configured to cause the central sling portion 12192′ to exert a force on the subject's abdominal wall skin AWS to urge the subject's abdominal wall soft tissue AST cephalad. The first, second, and third sling straps 13198a′, 13198b′, 13198c′ may thus be tensioned as desired to responsively urge the subject's abdominal wall soft tissue AST cephalad as desired.

At least one of the central sling portion 12192′, the first sling strap 13198a′, the second sling strap 13198b′, and the third sling strap 13198c′ may be formed separately from and then subsequently connected to at least one other of the central sling portion 12192′, the first sling strap 13198a′, the second sling strap 13198b′, and the third sling strap 13198c′. Alternatively or additionally, at least one of the central sling portion 12192′, the first sling strap 13198a′, the second sling strap 13198b′, and the third sling strap 13198c′ may be integrally formed with at least one other of the central sling portion 12192′, the first sling strap 13198a′, the second sling strap 13198b′, and the third sling strap 13198c′ as a single-piece, unitary construct.

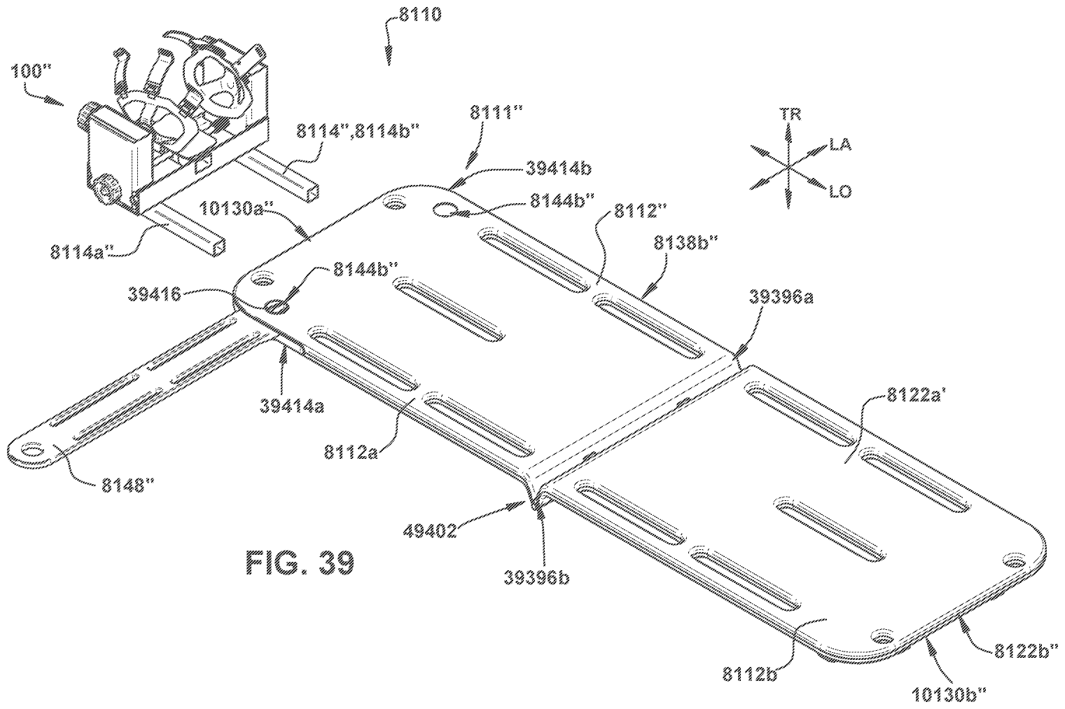

FIGS. 39-46 illustrate a third embodiment of a body stabilizer assembly 8111″ that may be included as part of the restraint assembly 8110. The body stabilizer assembly 8111″ of FIGS. 39-46 is similar to the body stabilizer assemblies 8111, 8111′ of FIGS. 8 and 10-12 and FIGS. 28-36 and therefore, structures of FIGS. 39-46 that are the same as or similar to those described with reference to FIGS. 8 and 10-12 and FIGS. 28-36 have the same reference numbers with the addition of a “double prime” mark. Description of common elements and operation similar to those in the previously described first embodiment may not be repeated with respect to the second embodiment, but should instead be considered to be incorporated below by reference as appropriate. Furthermore, elements and functionality shown or described with respect to one embodiment may be shared by any of the other embodiments whether or not expressly stated or shown.

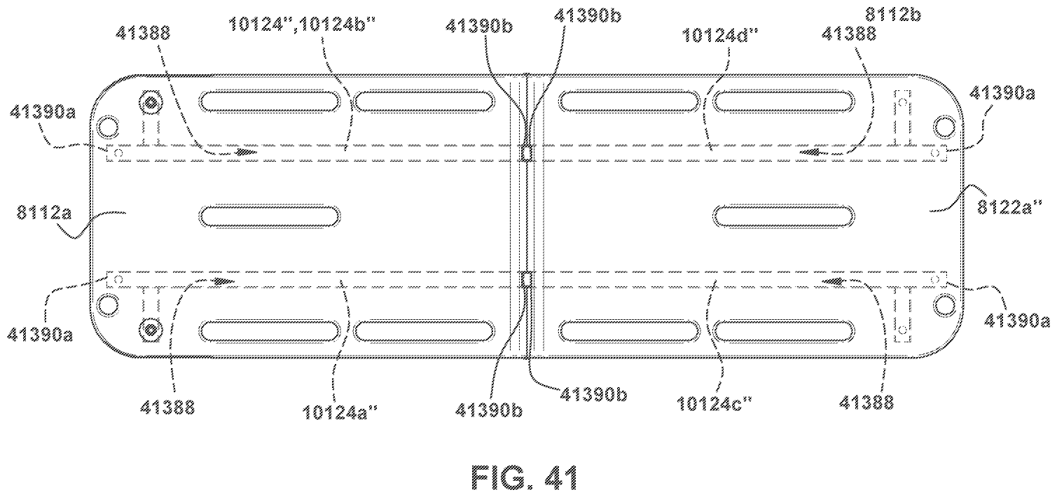

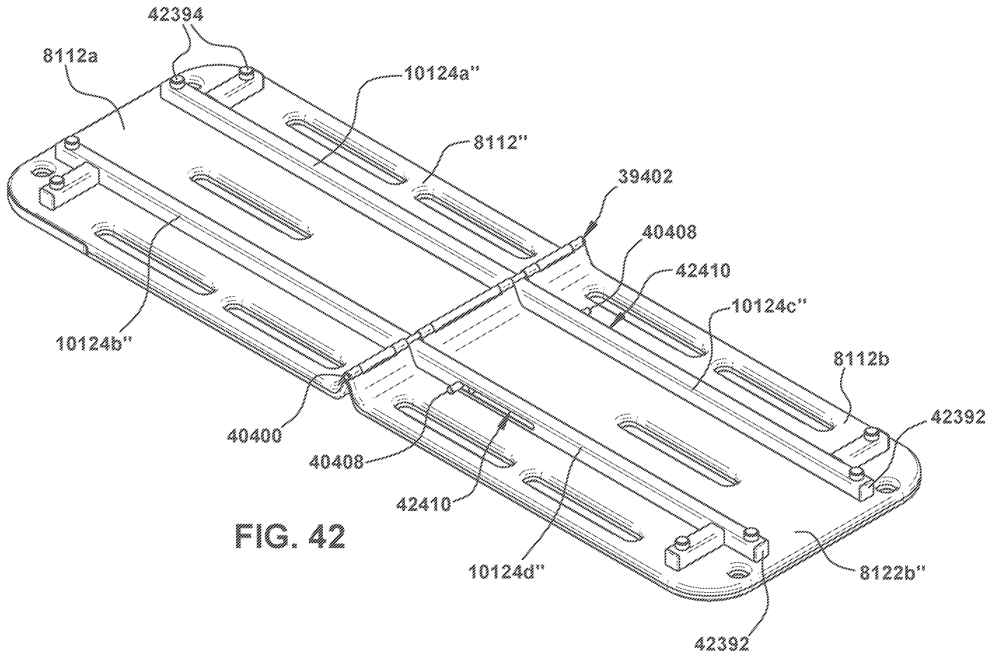

As shown in FIGS. 39-42, the body stabilizer 8112″ has transversely opposing first and second stabilizer surfaces 8122a″, 8122b″ that are separated from one another substantially in the transverse direction. The second stabilizer surface 8122b″ may include any other number of stabilizer bases 10124″ extending transversely therefrom, or any other stabilizing feature(s) extending therefrom for helping to stabilize the body stabilizer when in use. For example, as shown in FIGS. 41-42, the second stabilizer surface 8122b″ may include first and second stabilizer bases 10124a″, 10124b″ on a first body stabilizer portion 8112a, and third and fourth stabilizer bases 10124c″, 10124d″ on a second body stabilizer portion 8112b. Each stabilizer base 10124a″-d″ includes an inner base cavity 41388 that extends longitudinally between first and second base openings 41390a, 41390b. The second openings 41390b of the first and second stabilizer bases 10124a″, 10124b″ may extend substantially longitudinally through a portion of the first body stabilizer portion 8112a, while the second openings 41390b of the third and fourth stabilizer bases 10124c″, 10124d″ may extend substantially longitudinally through a portion of the second body stabilizer portion 8112b.

The first openings 41390a of the first through fourth stabilizer bases 10124a″-d″ are configured to selectively receive a head connector 8114″ (shown here as head connectors 8114a″-b″) for attaching any head restraint assembly 100, 100′ configuration to the body stabilizer 8112″. For example, as shown in FIG. 39, first and second head connectors 8114a″, 8114b″, which are shown in the form of tubes and/or bars having a square cross-sectional shape, may be removably or permanently connected to the restraint platform 102′. To attach the head restraint assembly 100′ to the body stabilizer 8112″, the first head connector 8114a″ is inserted into the inner base cavity 41388 of the first stabilizer base 10124a″, while the second head connector 8114b″ is inserted into the inner case cavity 41388 of the second stabilizer base 10124b″. As shown in FIG. 42, when the first and second stabilizer bases 10124a″-b″ are selected and/or configured to receive the first and second head connectors 8114a″, 8114b″, each of the first openings 41390a of the third and fourth stabilizer bases 10124c″-d″ may be selectively closed via a base cap 42392. Alternatively, if desired, the third and fourth stabilizer bases 10124c″-d″ may be selected to receive the first and second head connectors 8114a″, 8114b″, while each of the first openings 41390a of the first and second stabilizer bases 10124a″-b″ may be selectively closed via a base cap 42392.

Although the first and second head connectors 8114a″, 8114b″ are shown as being square in cross-section, it is contemplated that the first and second head connectors 8114a″, 8114b″ may have any desired cross-sectional shape such as, but not limited to, circular, elliptical, rectangular, curved, curvilinear, and/or rectilinear.

The inner base cavities 41388 may have a complementary shape to that of the first and second head connectors 8114a″, 8114b″. The inner base cavities 41388 may also be configured so that a press-fit and/or frictional engagement between the body stabilizer 8112″ and the first and second head connectors 8114a″, 8114b″ is created when the first and second head connectors 8114a″, 8114b″ are inserted into the selected inner base cavities 41388. The press-fit and/or frictional engagement between the body stabilizer 8112″ and the first and second head connectors 8114a″, 8114b″ may help prevent the first and second head connectors 8114a″, 8114b″ (and, accordingly, the head restraint assembly 100′) from being undesirably detached from the body stabilizer 8112″.

Alternatively, or additionally to the press-fit and/or frictional engagement maintaining the first and second head connectors 8114a″, 8114b″ connected to the body stabilizer 8112″, the first and second head connectors 8114a″, 8114b″ may be substantially permanently connected or selectively connected to the body stabilizer 8112″ after being inserted into the inner base cavities 41388 via one or more fasteners. Each fastener that connects the first and second head connectors 8114a″, 8114b″ to the body stabilizer 8112″ may be at least one of an adhesive (such as a permanent adhesive epoxy), tape (such as a high performance acrylic adhesive double-sided tape), a screw (such as a plastic or carbon fiber screw), nail (such as a plastic or carbon fiber nail), a hook and loop fastener, a snap fastener, any suitable metallic or non-metallic fastener, and/or any other suitable fastener or combination of fasteners. It is also contemplated that the first and second head connectors 8114a″, 8114b″ may be integrally formed as a single-piece, unitary construct with the body stabilizer 8112″.

As shown in FIG. 42, the first through fourth stabilizer bases 10124a″-d″ may each include one or more base feet 42394 connected (e.g., bonded) thereto for helping to stabilize and/or level the body stabilizer 8112″ when in use. The base feet 42394, when present, may be at least partially formed from polyoxymethylene (otherwise known as acetal) or any other desired material.

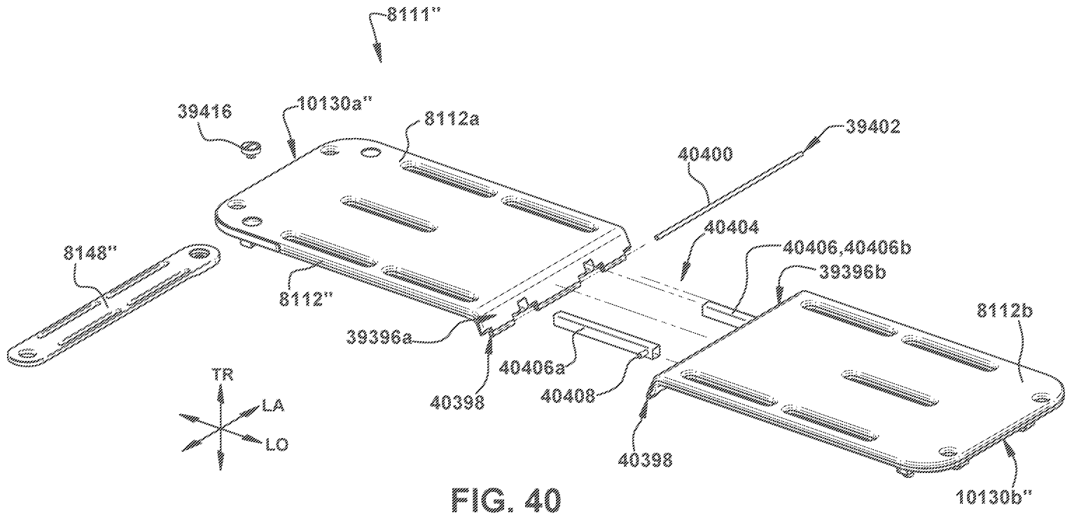

As shown in FIGS. 39-40, the first body stabilizer portion 8112a extends longitudinally from the first longitudinal stabilizer side 10130a″ of the body stabilizer 8112″ to a first hinge portion 39396a. Similarly, the second body stabilizer portion 8112b extends longitudinally from the second longitudinal stabilizer side 10130b″ to a second hinge portion 39396b. Each of the first and second hinge portions 39396a, 39396b includes at least one hinge opening 40398. The first and second hinge portions 39396a, 39396b are configured to be selectively engaged (e.g., interdigitated) to one another such that the hinge openings 40398 of the first and second hinge portions 39396a, 39396b are all axially aligned with one another. Once the hinge openings 40398 are all aligned, a hinge bar 40400 may be inserted through each of the hinge openings 40398 to selectively couple the first and second hinge portions 39396a, 39396b (and, thus, the first and second body stabilizer portions 8112a, 8112b) together and form the body stabilizer 8112″. Therefore, via the first and second hinge portions 39396a, 39396b and the hinge bar 40400, the first and second body stabilizer portions 8112a, 8112b may be selectively coupled and decoupled from one another.

The first and second hinge portions 39396a, 39396b and the hinge bar 40400 thus define a hinge mechanism 39402 by which the first and second body stabilizer portions 8112a, 8112b may be selectively coupled to one another and selectively pivoted about the hinge bar 40400 relative to one another (or one relative to the other) between an unfolded condition (FIGS. 39, 41-44) and a folded condition (FIG. 45). In the unfolded condition, the body stabilizer 8112″ is configured to receive at least a portion of the subject S. As shown in FIGS. 43-45, a longitudinal length of the body stabilizer 8112″ is greater when in the unfolded condition than when in the folded condition. Because the body stabilizer 8112″ is smaller in the folded condition (and, thus takes up less longitudinal space), the body stabilizer 8112″ may be manipulated into the folded condition for storage. As shown in FIG. 45, the second stabilizer surface 8122b″ at the first body stabilizer portion 8112a faces the second stabilizer surface 8122b″ at the second body stabilizer portion 8112b substantially in the transverse direction when the body stabilizer 8112″ is folded.

To prevent the body stabilizer 8112″ from undesirably pivoting from the unfolded condition to the folded condition, such as during use, the body stabilizer 8112″ may include a pivot locking mechanism 40404. As shown in FIG. 40 the pivot locking mechanism 40404 includes at least one locking bar 40406 (shown here as first and second locking bars 40406a-b) extending substantially in the longitudinal direction. The first locking bar 40406a is configured to be at least partially received in the inner base cavity 41388 of the third stabilizer base 10124c″, while the second locking bar 40406b is configured to be at least partially received in the inner base cavity 41388 of the fourth stabilizer base 10124d″. As shown in FIGS. 40 and 42, the first and second locking bars 40406a, 40406b each have a locking projection 40408 extending laterally therefrom along which the first and second locking bars 40406a, 40406b may be selectively slid relative to their respective inner base cavities 41388. Each locking projection 40408 extends laterally through a corresponding guiding opening 42410 in a corresponding one of the third and fourth stabilizer bases 10124c″-d″.

Via their respective locking projections 40408, the first and second locking bars 40406a, 40406b may be selectively slid between locked and unlocked positions. FIGS. 41-43 depict the body stabilizer 8112″ in the unfolded condition in which the second openings 40398b (and, accordingly, the inner base cavities 41388) of the first and third stabilizer bases 10124a″, 10124c″ are axially aligned with one another, and the second openings 40398b (and, accordingly, the inner base cavities 41388) of the second and fourth stabilizer bases 10124b″, 10124d″ are axially aligned with one another. FIGS. 42-43 also show the first and second locking bars 40406a, 40406b in the locked position. The first and second locking bars 40406a, 40406b extend from the inner base cavities 41388 of the third and fourth stabilizer bases 10124c″, 10124d″ into the inner base cavities 41388 the first and second stabilizer bases 10124a″, 10124b″ when in the locked position. By spanning and being at least partially located in the inner base cavities 41388 of the first and third stabilizer bases 10124a″, 10124c″ and the second and fourth stabilizer bases 10124b″, 10124d″, the first and second locking bars 40406a, 40406b substantially prevent the first and second body stabilizer portions 8112a, 8112b from being undesirably pivoted from the unfolded condition.

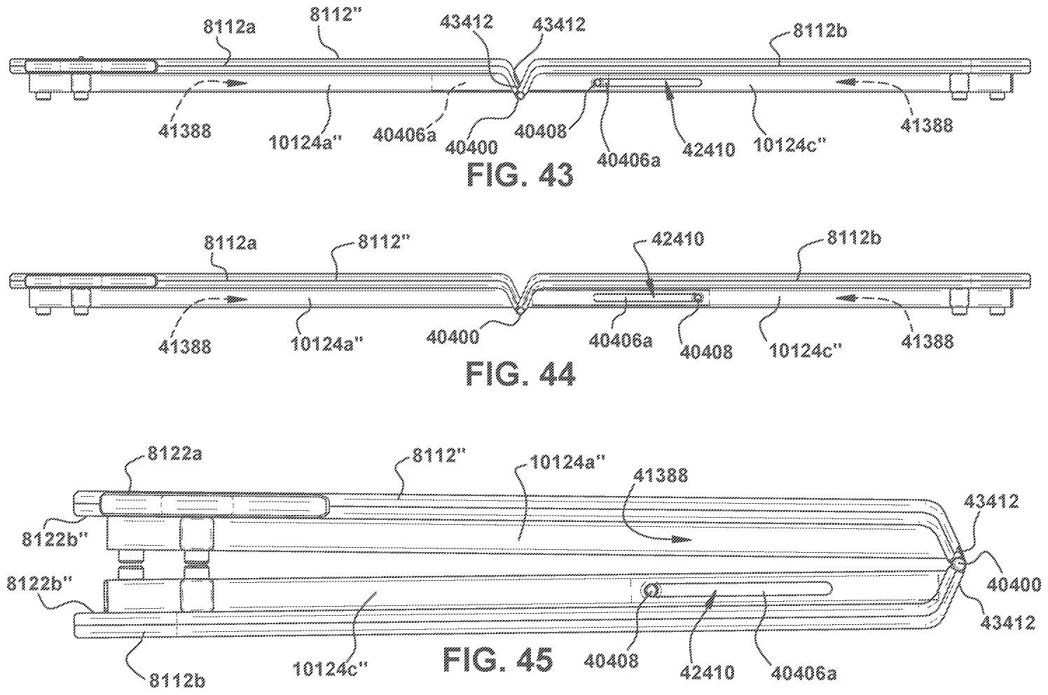

When the medical professional desires to transition the body stabilizer 8112″ from the unfolded condition to the folded condition, the medical professional may longitudinally slide the locking projections 40408 along their respective guiding openings 40410 to responsively slide the first and second locking bars 40406a, 40406b from the locked position (FIG. 43) to the unlocked position (FIG. 44). The first and second locking bars 40406a, 40406b are slid entirely out from the inner base cavities 41388 of the first and second stabilizer bases 10124a″, 10124b″ as the first and second locking bars 40406a, 40406b are moved to the unlocked condition. As shown in FIG. 44, the first and second locking bars 40406a, 40406b thus are substantially (if not entirely) located within the inner base cavities 41388 of the third and fourth stabilizer bases 10124c″, 10124d″ when in the unlocked position. Once the first and second locking bars 40406a, 40406b are in the unlocked position, the medical professional may pivot the first and second stabilizer body portions 8112a, 8112b relative to one another (or one relative to the other) about the hinge bar 40400 from the unfolded position shown in FIG. 44 to the folded position shown in FIG. 45.

When desired, the medical professional may once again pivot the first and second body stabilizer portions 8112a, 8112b from the folded position to the unfolded position. A shown in FIGS. 43-45, the first and second hinge portions 39396a, 39396b may include complementary stop surfaces 43412 that are configured to engage one another and substantially prevent the first and second body stabilizer portions 8112a, 8112b from being pivoted beyond the unfolded condition when being transitioned back to the unfolded condition. However, it is contemplated that the first and second hinge portions 39396a, 39396b may have any other stop features (e.g., projections) in addition to or instead of the stop surfaces 43412.

As shown in FIGS. 39 and 46-47, at least one of the first and second lateral stabilizer sides 8138a″, 8138b″ may include a winglet attachment aperture 8144a″, 8144b″ extending transversely therethrough. For example, the body stabilizer 8112″ shown in FIGS. 39 and 46-47 includes a first winglet attachment aperture 8144a″ on the first lateral stabilizer side 8138a″ and a second winglet attachment aperture 8144b″ on the second lateral stabilizer side 8138b″. Each of the winglet attachment apertures 8144a″, 8144b″ are shown as being on the first stabilizer body portion 8114a, though the body stabilizer 8112″ may be configured such that winglet attachment apertures are also or instead on the second stabilizer body portion 8114b.

Instead of extending entirely transversely through both the first and second stabilizer surfaces 8122a″, 8122b″, the first and second winglet attachment apertures 8144a″, 8144b″ extend transversely through only the first stabilizer surface 8122a″. Each of the first and second winglet attachment apertures 8144a″, 8144b″ are in fluid communication with a winglet attachment slot 39414a, 39414b of the body stabilizer 8112″. In particular, the first winglet attachment aperture 8144a″ extends transversely to and is in fluid communication with a first winglet attachment slot 39414a. The first winglet attachment slot 39414a is on the first lateral stabilizer side 8138a″ and extends at least partially in the lateral direction toward the second lateral stabilizer side 8138b″. Similarly, the second winglet attachment aperture 8144b″ extends transversely to and is in fluid communication with a second winglet attachment slot 39414b. The second winglet attachment slot 39414b is on the second lateral stabilizer side 8138b″ and extends at least partially in the lateral direction toward the first lateral stabilizer side 8138a″.

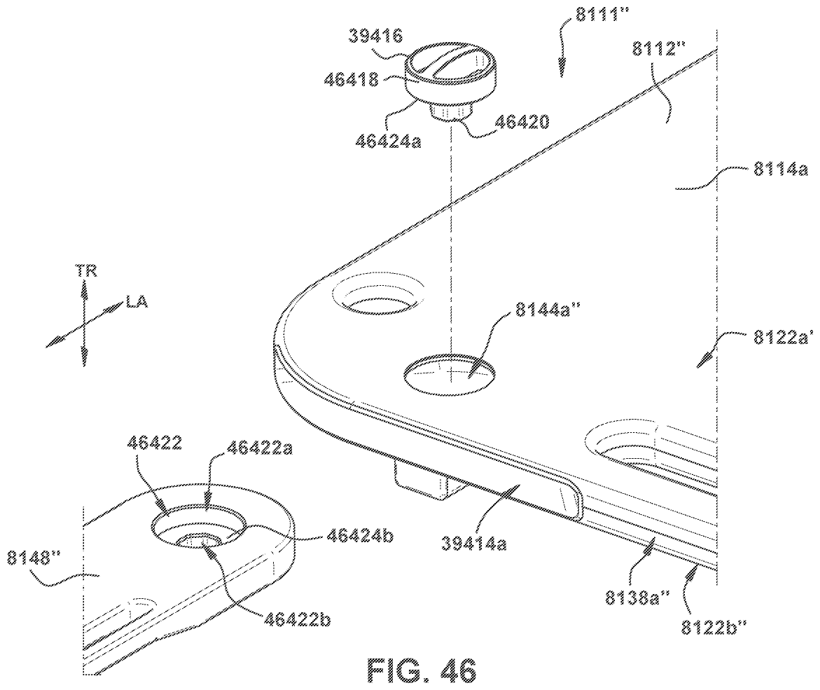

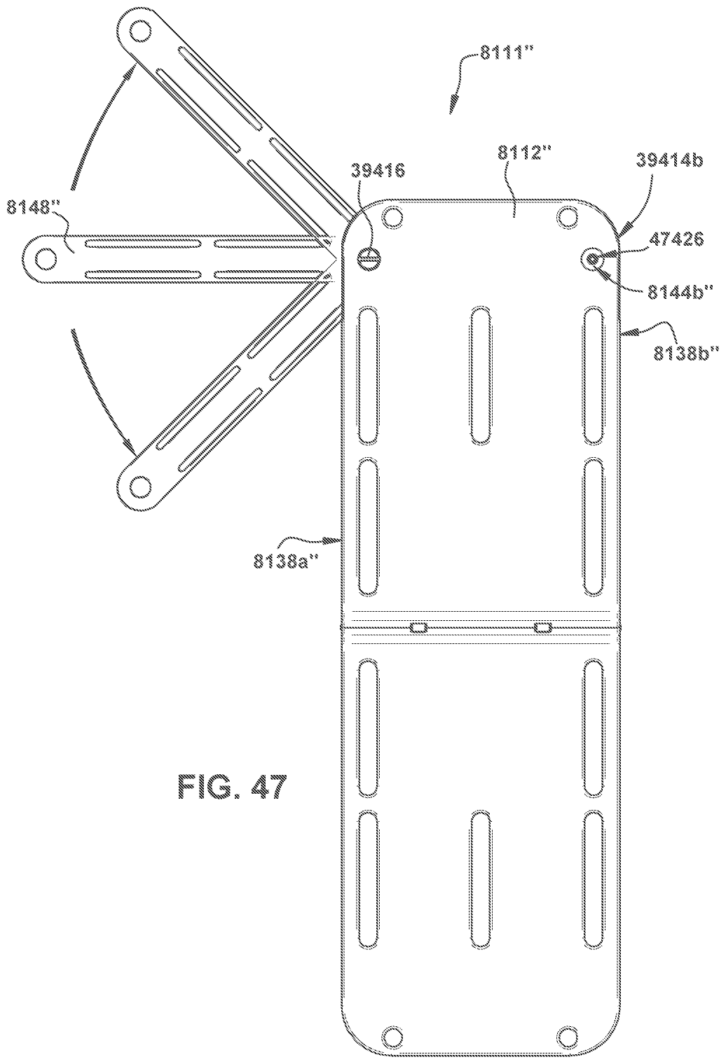

Each of the first and second winglet attachment slots 39414a, 39414b is configured to selectively receive a winglet 8148″ and permit the winglet 8148″ to be selectively pivoted relative to the body stabilizer 8112″ to a desired angular position for receiving the subject's arm A. The body stabilizer assembly 8111″ may include a winglet lock 39416 for selectively retaining the winglet 8148″ at the desired angular position. As shown in FIG. 46, the winglet lock 39416 includes a lock body 46418, which may be circular in cross-section, and a lock pin 46420, which may be hexagonal in cross-section. The winglet 8148″ has a winglet depression 46422 for receiving at least a portion of the winglet lock 39416. The winglet depression 46422 may have a first depression portion 46422a configured to receive the lock body 46418 and a second depression portion 46422b configured to receive the lock pin 46420. The first depression portion 46422a thus is circular in cross-section, while the second depression portion 46422b is hexagonal in cross-section.

In use, the medical professional may insert the winglet 8148″ into, for example, the first winglet attachment slot 39414a and then position the winglet 8148″ in the first winglet attachment slot 39414a such that the winglet depression 46422 is axially aligned with the first winglet attachment aperture 8144a″. Once aligned, the winglet 8148″ may be pivoted to the desired angular position. The winglet lock 39416 may then be inserted through the first winglet attachment aperture 8144a″ in the transverse direction until a first transverse engagement surface 46424a of the lock body 46418 contacts a second transverse engagement surface 46424b of the first depression portion 46422a. When the first and second transverse engagement surfaces 46424a, 46424b contact one another, the lock body 46418 is at least partially positioned in the first depression portion 46422a and the lock pin 46420 is at least partially positioned in the second depression portion 46422b. Once inserted, the winglet lock 39416 substantially prevents the winglet 8148″ from undesirably egressing the first winglet attachment slot 39414a and from undesirably pivoting from the desired angular position. If the medical professional desires to remove or pivot the winglet 8148″, the winglet lock 39416 may be at least partially removed from the winglet depression 46422 and the first winglet attachment aperture 8144a″.

Because the lock pin 46420 and the second depression portion 46422b are both hexagonal, the lock pin 46420 and the second depression portion 46422b are keyed or indexed such that the winglet 8148″ is pivotable at predetermined angular increments (e.g., 45-degree increments) relative to the body stabilizer 8112″ (as shown in the configuration of FIG. 46-47). The lock pin 46420 and/or the second depression portion 46422b, however, may be keyed or indexed in any other manner to achieve the incremental pivoting. For example, instead of being substantially hexagonal, the lock pin 46420 and/or the second depression portion 46422b may be substantially shaped as any other polygon or any other desired shape. Furthermore, instead of being circular in cross-section, the lock body 46418 may have any desired cross-sectional shape, such as, but not limited to, elliptical, rectangular, curved, curvilinear, and/or rectilinear.

An end portion 47426 (FIG. 47) of each of the first and second winglet attachment apertures 8144a″, 8144b″ may be configured to selectively receive a portion of the lock pin 46420 when the winglet lock 39416 is inserted into the first and second winglet attachment apertures 8144a″, 8144b″. In particular, the lock pin 46420 may extend through the winglet depression 46422 and into a selected end portion 47426 when coupling the winglet 8148″ to the body stabilizer 8112″. Each end portion 47426 may be configured such that when the lock pin 46420 is located therein, the lock pin 46420 (and, accordingly, the attached winglet 8148″) is substantially prevented from undesirably rotating. The end portions 47426 may have any desired cross-sectional shape (such as, but not limited to, hexagonal, rectangular, curved, curvilinear, and/or rectilinear) for selectively receiving the lock pin 46420 and for selectively preventing the lock pin 46420 from rotating. In the example configuration shown in FIG. 47, the end portion 47426 has a hexagonal cross-sectional shape.

Although the body stabilizer assembly 8111″ is shown as having only one winglet 8148″, the body stabilizer assembly 8111″ may have any number of winglets 8148″. For example, one winglet 8148″ may be inserted in the first winglet attachment slot 39414a, while a second winglet 8148″ may be inserted in the second winglet attachment slot 39414b.

The individual pieces of the restraint assembly can each be at least partially formed from fabric, nylon, foam, rubber, silicone, polyethylene (such as rotationally molded high density polyethylene), thermoplastic polymer(s), polyvinyl chloride (“PVC”), an expanded closed cell PVC sheet, glass reinforced thermoplastic polymer(s) (e.g., glass reinforced acrylonitrile butadiene styrene), stainless steel, titanium, carbon fiber, polyoxymethylene (acetal), any other desired material, and/or any combination thereof. However, in some use environments, the head restraint assembly, body stabilizer assembly, any other portion of the restraint assembly, and/or the restraint assembly in its entirety may constructed without metal or any other non-MRI scanner compliant material to restrain the subject S during an MRI scan.

A person having ordinary skill in the art would understand that, in an example aspect 1, a head restraint assembly comprises:

a restraint platform configured to receive a head of a subject;

a first head restraint attached to the restraint platform and configured to selectively engage a first side of the subject's head, the first head restraint having a first cheekbone flange configured to selectively engage a cheekbone region of the subject's head on the first side of the subject's head;

a second head restraint attached to the restraint platform and configured to selectively engage a second side of the subject's head, the second head restraint having a second cheekbone flange configured to selectively engage a cheekbone region of the subject's head on the second side of the subject's head; and

a cheekbone strap extending between the first and second cheekbone flanges and configured for selectively tightening the cheekbone flanges against the subject's cheekbone regions, the cheekbone strap being configured to be spaced from the subject's head when the subject's head is restrained by the head restraint assembly.

Example aspect 2. The head restraint assembly of example aspect 1, further comprising:

a first ball-and-socket assembly having a first ball portion attached to one of the first head restraint and a first ball-and-socket base and a first socket portion on the other one of the first head restraint and the first ball-and-socket base, the first ball portion being selectively received in the first socket portion and thereby providing the first head restraint with at least three degrees of freedom of movement for the first head restraint to conform to the first side of the subject's head; and

a second ball-and-socket assembly having a second ball portion attached to one of the second head restraint and a second ball-and-socket base and a second socket portion on the other one of the second head restraint and the second ball-and-socket base, the second ball portion being selectively received in the second socket portion and thereby providing the second head restraint with at least three degrees of freedom of movement for the second head restraint to conform to the second side of the subject's head.

Example aspect 3. The head restraint assembly of example aspect 2, wherein

the first ball-and-socket assembly has a first ball inhibiting mechanism for selectively engaging the first ball portion to inhibit movement of the first ball portion relative to the first ball-and-socket base, the first head restraint being retained in a fixed position as a result of the first ball inhibiting mechanism inhibiting the movement of the first ball portion, and

the second ball-and-socket assembly has a second ball inhibiting mechanism for selectively engaging the second ball portion to inhibit movement of the second ball portion relative to the second ball-and-socket base, the second head restraint being retained in a fixed position as a result of the second ball inhibiting mechanism inhibiting the movement of the second ball portion.

Example aspect 4. The head restraint assembly of example aspect 1, wherein

the first head restraint includes a first forehead flange configured to selectively engage a forehead of the subject's head on the first side of the subject's head,

the second head restraint includes a second forehead flange configured to selectively engage the subject's forehead on the second side of the subject's head,

the first head restraint includes a first crown flange configured to selectively engage a crown of the subject's head on the first side of the subject's head, and

the second head restraint includes a second crown flange configured to selectively engage the subject's crown on the second side of the subject's head, the head restraint assembly further comprising:

a forehead strap extending between the first and second forehead flanges and configured for selectively tightening the forehead flanges against the subject's forehead; and

and a crown strap extending between the first and second crown flanges and configured for selectively tightening the crown flanges against the subject's crown.

Example aspect 5. The head restraint assembly of example aspect 4, wherein

the first head restraint includes a first base portion for engaging the first side of the subject's head, each of the first cheekbone flange, the first forehead flange, and the first crown flange being connected to the first base portion, the first base portion having a first earhole defined by an inner periphery of the first base portion configured to be substantially aligned with at least a portion of an ear of the subject when the first head restraint is engaged to the first side of subject's head, and

the second head restraint includes a second base portion for engaging the second side of the subject's head, each of the second cheekbone flange, the second forehead flange, and the second crown flange being connected to the second base portion, the second base portion having a second earhole defined by an inner periphery of the second base portion configured to be substantially aligned with at least a portion of an ear of the subject when the second head restraint is engaged to the second side of subject's head.

Example aspect 6. The head restraint assembly of example aspect 5, further comprising:

a first cushion interposed between the first side of the subject's head and at least one of the first base portion, the first cheekbone flange, the first forehead flange, and the first crown flange; and

a second cushion interposed between the second side of the subject's head and at least one of the second base portion, the second cheekbone flange, the second forehead flange, and the second crown flange.

Example aspect 7. The head restraint assembly of example aspect 1, wherein at least one of the first and second head restraints are selectively movable in a lateral direction with respect to the restraint platform and the other of the first and second head restraints.

Example aspect 8. A restraint assembly for helping to restrain a subject, comprising:

the head restraint assembly of example aspect 1; and

a body stabilizer configured to receive a portion of the subject's body below the subject's head, the body stabilizer being removably attached to the head restraint assembly.

Example aspect 9. The restraint assembly of example aspect 8, further comprising an abdominal sling having a central sling portion configured to selectively engage the subject's abdominal wall skin, the abdominal sling having at least one sling strap extending from the central sling portion and configured to be selectively attached to the body stabilizer,

wherein the abdominal sling is configured such that, when the central device portion engages the subject's abdominal wall skin and the at least one sling strap is attached to the body stabilizer, the central sling portion exerts a force on the subject's abdominal wall skin to urge the subject's abdominal wall soft tissue cephalad.

Example aspect 10. The restraint assembly of example aspect 9, further comprising at least one restraining strap configured to be selectively attached to the body stabilizer and selectively prevent the egress of the subject from the body stabilizer,

wherein the at least one sling strap includes a first sling strap extending from the central sling portion and configured to be selectively attached to the body stabilizer on a first side of the body stabilizer, a second sling strap extending from the central sling portion and configured to be selectively attached to the body stabilizer on a second side of the body stabilizer, and a third sling strap extending from the central sling portion and configured to be selectively attached to the at least one restraining strap.

Example aspect 11. The restraint assembly of example aspect 8, wherein the body stabilizer comprises separate first and second body stabilizer portions that are selectively coupled to one another via a hinge mechanism, at least one of the first and second body stabilizer portions being pivotable relative to the other of the first and second body stabilizer portions between an unfolded and folded conditions via the hinge mechanism.

Example aspect 12. The restraint assembly of example aspect 11, wherein the body stabilizer comprises a pivot locking mechanism for selectively locking the first and second body stabilizer portions in the unfolded condition.

Example aspect 13. A head restraint assembly, comprising:

a restraint platform configured to receive a head of a subject;

a first head restraint attached to the restraint platform and configured to selectively engage a first side of the subject's head;

a second head restraint attached to the restraint platform and configured to selectively engage a second side of the subject's head;

a first articulating assembly having a first portion attached to the first head restraint and a second portion connected to the restraint platform, the first portion of the first articulating assembly being selectively articulatable relative to the second portion of the first articulating assembly and thereby providing the first head restraint with at least three degrees of freedom of movement for the first head restraint to conform to the first side of the subject's head; and

a second articulating assembly having a first portion attached to the second head restraint and a second portion connected to the restraint platform, the first portion of the second articulating assembly being selectively articulatable relative to the second portion of the second articulating assembly and thereby providing the second head restraint with at least three degrees of freedom of movement for the second head restraint to conform to the second side of the subject's head.

Example aspect 14. The head restraint assembly of example aspect 13, wherein

the first head restraint includes a first cheekbone flange configured to selectively engage a cheekbone region of the subject's head on the first side of the subject's head, and

the second head restraint includes a second cheekbone flange configured to selectively engage a cheekbone region of the subject's head on the second side of the subject's head, the head restraint assembly further comprising:

a cheekbone strap extending between the first and second cheekbone flanges and configured for selectively tightening the cheekbone flanges against the subject's cheekbone regions, the cheekbone strap being configured to be spaced from the subject's head when the subject's head is restrained by the head restraint assembly.

Example aspect 15. The head restraint assembly of example aspect 14, wherein at least one of the first and second cheekbone flanges includes a notch configured to selectively receive and selectively retain a medical tubing therein.

Example aspect 16. The head restraint assembly of example aspect 14, wherein

the first head restraint includes a first forehead flange configured to selectively engage a forehead of the subject's head on the first side of the subject's head, and

the second head restraint includes a second forehead flange configured to selectively engage the subject's forehead on the second side of the subject's head, the head restraint assembly further comprising:

a forehead strap extending between the first and second forehead flanges and configured for selectively tightening the forehead flanges against the subject's forehead.

Example aspect 17. The head restraint assembly of example aspect 16, wherein

the first head restraint includes a first crown flange configured to selectively engage a crown of the subject's head on the first side of the subject's head, and

the second head restraint includes a second crown flange configured to selectively engage the subject's crown on the second side of the subject's head, the head restraint assembly further comprising:

a crown strap extending between the first and second crown flanges and configured for selectively tightening the crown flanges against the subject's crown.

Example aspect 18. The head restraint assembly of example aspect 17, wherein

the first head restraint includes a first base portion for engaging the first side of the subject's head, each of the first cheekbone flange, the first forehead flange, and the first crown flange being connected to the first base portion, the first base portion having a first earhole defined by an inner periphery of the first base portion configured to be substantially aligned with at least a portion of an ear of the subject when the first head restraint is engaged to the first side of subject's head, and

the second head restraint includes a second base portion for engaging the second side of the subject's head, each of the second cheekbone flange, the second forehead flange, and the second crown flange being connected to the second base portion, the second base portion having a second earhole defined by an inner periphery of the second base portion configured to be substantially aligned with at least a portion of an ear of the subject when the second head restraint is engaged to the second side of subject's head.

Example aspect 19. The head restraint assembly of example aspect 18, further comprising:

a first cushion interposed between the first side of the subject's head and at least one of the first base portion, the first cheekbone flange, the first forehead flange, and the first crown flange; and

a second cushion interposed between the second side of the subject's head and at least one of the second base portion, the second cheekbone flange, the second forehead flange, and the second crown flange.

Example aspect 20. A restraint assembly for helping to restrain a subject, comprising:

the head restraint assembly of example aspect 13; and

a body stabilizer configured to receive a portion of the subject's body below the subject's head, the body stabilizer being removably attached to the head restraint assembly.

Example aspect 21. The restraint assembly of example aspect 20, further comprising an abdominal sling having a central sling portion configured to selectively engage the subject's abdominal wall skin, the abdominal sling having at least one sling strap extending from the central sling portion,

wherein the abdominal sling is configured such that when the central sling portion engages the subject's abdominal wall skin and the at least one sling strap is tensioned, the central sling portion exerts a force on the subject's abdominal wall skin to urge the subject's abdominal wall soft tissue cephalad.

Example aspect 22. The restraint assembly of example aspect 21, further comprising at least one restraining strap configured to be selectively attached to the body stabilizer and selectively prevent the egress of the subject from the body stabilizer,

wherein the at least one sling strap includes a first sling strap extending from the central sling portion and configured to be selectively attached to the body stabilizer on a first side of the body stabilizer, a second sling strap extending from the central sling portion and configured to be selectively attached to the body stabilizer on a second side of the body stabilizer, and a third sling strap extending from the central sling portion and configured to be selectively attached to the at least one restraining strap.

Example aspect 23. The restraint assembly of example aspect 20, wherein the body stabilizer comprises separate first and second body stabilizer portions that are selectively coupled to one another via a hinge mechanism, at least one of the first and second body stabilizer portions being pivotable relative to the other of the first and second body stabilizer portions between an unfolded and folded conditions via the hinge mechanism.

Example aspect 24. The restraint assembly of example aspect 23, wherein the body stabilizer comprises a pivot locking mechanism for selectively locking the first and second body stabilizer portions in the unfolded condition.

Example aspect 25. An abdominal sling, comprising:

a central sling portion configured to selectively engage the subject's abdominal wall skin; and

at least one sling strap extending from the central sling portion and configured to be selectively tensioned;

wherein the abdominal sling is configured such that, when the central device portion engages the subject's abdominal wall skin and the at least one sling strap is tensioned, the central sling portion exerts a force on the subject's abdominal wall skin to urge the subject's abdominal wall soft tissue cephalad.

Example aspect 26. The abdominal sling of example aspect 25, wherein the at least one sling strap includes a plurality of sling straps, each of the sling straps extending from the central sling portion and being configured to be selectively tensioned.

Example aspect 27. A restraint assembly for helping to restrain a subject, comprising:

a body stabilizer configured to receive a portion of the subject's body; and

the abdominal sling of example aspect 25.

Example aspect 28. The restraint assembly of example aspect 27, further comprising at least one winglet configured to receive an arm of the subject, the at least one winglet being attached to the body stabilizer and pivotable relative to the body stabilizer.

Example aspect 29. The restraint assembly of example aspect 27, further comprising a foot platform configured to receive at least the subject's feet and be removably attached to the body stabilizer.

Example aspect 30. The restraint assembly of example aspect 27, further comprising a head restraint assembly configured to receive at least the subject's head and be removably attached to the body stabilizer.

Example aspect 31. The restraint assembly of example aspect 27, further comprising at least one restraining strap configured to be selectively attached to the body stabilizer and selectively prevent the egress of the subject from the body stabilizer,

wherein the at least one sling strap includes a first sling strap extending from the central sling portion and configured to be selectively attached to the body stabilizer on a first side of the body stabilizer, a second sling strap extending from the central sling portion and configured to be selectively attached to the body stabilizer on a second side of the body stabilizer, and a third sling strap extending from the central sling portion and configured to be selectively attached to the at least one restraining strap.

Example aspect 32. The restraint assembly of example aspect 27, wherein the body stabilizer comprises separate first and second body stabilizer portions that are selectively coupled to one another via a hinge mechanism, at least one of the first and second body stabilizer portions being pivotable relative to the other of the first and second body stabilizer portions between an unfolded and folded conditions via the hinge mechanism.

Example aspect 33. The restraint assembly of example aspect 32, wherein the body stabilizer comprises a pivot locking mechanism for selectively locking the first and second body stabilizer portions in the unfolded condition.

Example aspect 34. A body stabilizer assembly for helping to restrain a subject, comprising:

a body stabilizer configured to receive a portion of the subject, the body stabilizer including at least one stabilizer aperture longitudinally positioned along a first lateral stabilizer side of the body stabilizer and at least one stabilizer aperture longitudinally positioned along an opposite second lateral stabilizer side of the body stabilizer, each stabilizer aperture extending transversely through the body stabilizer;

a plurality of strap connectors, each strap connector being at least partially positioned in an associated stabilizer aperture and selectively longitudinally moveable along the associated stabilizer aperture; and

at least one restraining strap configured to be selectively attached to the body stabilizer via the strap connectors and selectively prevent the egress of the subject from the body stabilizer, each end of the at least one restraining strap being selectively connected to an associated strap connector;

wherein via the strap connectors, the at least one restraining strap is selectively longitudinally moveable relative to the body stabilizer to correspond to the subject's anatomy.

Example aspect 35. The body stabilizer assembly of example aspect 34, wherein each strap connector includes a bar, each end of the at least one restraining strap has a hook connected thereto, each strap connector includes a bar to which an associated hook may be selectively attached.

Example aspect 36. The body stabilizer assembly of example aspect 34, wherein the at least one restraining strap comprises a plurality of restraining straps, and wherein the body stabilizer including a plurality of stabilizer apertures longitudinally positioned along the first lateral stabilizer side of the body stabilizer and a plurality of stabilizer apertures longitudinally positioned along the second lateral stabilizer side of the body stabilizer, each stabilizer aperture having at least one of the strap connectors at least partially positioned therein.

Example aspect 37. The body stabilizer assembly of example aspect 36, wherein the body stabilizer includes a stabilizer aperture positioned laterally between the stabilizer apertures of the first lateral stabilizer side and the stabilizer apertures of the second lateral stabilizer side.

Example aspect 38. The body stabilizer assembly of example aspect 34, wherein the body stabilizer is configured to receive a portion of the subject's body below the subject's head.

Example aspect 39. A restraint assembly for helping to restrain a subject, comprising:

the body stabilizer assembly of example aspect 34; and

a head restraint assembly configured to receive a portion of the subject's head, the head restraint assembly being removably attached to the body stabilizer.

Example aspect 40. The restraint assembly of example aspect 39, further comprising an abdominal sling having a central sling portion configured to selectively engage the subject's abdominal wall skin, the abdominal sling having at least one sling strap extending from the central sling portion and configured to be selectively attached to the body stabilizer,

wherein the abdominal sling is configured such that, when the central device portion engages the subject's abdominal wall skin and the at least one sling strap is attached to the body stabilizer, the central sling portion exerts a force on the subject's abdominal wall skin to urge the subject's abdominal wall soft tissue cephalad.

Example aspect 41. The restraint assembly of example aspect 40, wherein the at least one sling strap includes a first sling strap extending from the central sling portion and configured to be selectively attached to the body stabilizer on a first side of the body stabilizer, a second sling strap extending from the central sling portion and configured to be selectively attached to the body stabilizer on a second side of the body stabilizer, and a third sling strap extending from the central sling portion and configured to be selectively attached to the at least one restraining strap.

Example aspect 42. The restraint assembly of example aspect 34, wherein the body stabilizer comprises separate first and second body stabilizer portions that are selectively coupled to one another via a hinge mechanism, at least one of the first and second body stabilizer portions being pivotable relative to the other of the first and second body stabilizer portions between an unfolded and folded conditions via the hinge mechanism.

Example aspect 43. The restraint assembly of example aspect 42, wherein the body stabilizer comprises a pivot locking mechanism for selectively locking the first and second body stabilizer portions in the unfolded condition.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a medical professional in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

I claim:

1. A head restraint assembly, comprising:
a restraint platform configured to receive a head of a subject;
a first head restraint attached to the restraint platform and configured to selectively engage a first side of the subject's head, the first head restraint having a first cheekbone flange configured to selectively engage a cheekbone region of the subject's head on the first side of the subject's head;
a second head restraint attached to the restraint platform and configured to selectively engage a second side of the subject's head, the second head restraint having a second cheekbone flange configured to selectively engage a cheekbone region of the subject's head on the second side of the subject's head; and
a cheekbone strap extending between the first and second cheekbone flanges and configured for selectively tightening the cheekbone flanges against the subject's cheekbone regions, the cheekbone strap being configured to be spaced from the subject's head when the subject's head is restrained by the head restraint assembly.

2. The head restraint assembly of claim 1, further comprising:
a first ball-and-socket assembly having a first ball portion attached to one of the first head restraint and a first ball-and-socket base and a first socket portion on the other one of the first head restraint and the first ball-and-socket base, the first ball portion being selectively received in the first socket portion and thereby providing the first head restraint with at least three degrees of freedom of movement for the first head restraint to conform to the first side of the subject's head; and
a second ball-and-socket assembly having a second ball portion attached to one of the second head restraint and a second ball-and-socket base and a second socket portion on the other one of the second head restraint and the second ball-and-socket base, the second ball portion being selectively received in the second socket portion and thereby providing the second head restraint with at least three degrees of freedom of movement for the second head restraint to conform to the second side of the subject's head.

3. The head restraint assembly of claim 2, further comprising:
a first sliding member configured to be selectively slid laterally along the restraint platform, the first sliding member being fixed to the first ball-and-socket base, selective lateral movement of the first sliding member responsively causing the first head restraint to move laterally relative to the restraint platform; and
a second sliding member configured to be selectively slid laterally along the restraint platform, the second sliding member being fixed to the second ball-and-socket base, selective lateral movement of the second sliding member responsively causing the second head restraint to move laterally relative to the restraint platform.

4. The head restraint assembly of claim 1, wherein at least one of the first and second cheekbone flanges includes a notch configured to selectively receive and selectively retain a medical tubing therein.

5. The head restraint assembly of claim 1, wherein
the first head restraint includes a first forehead flange configured to selectively engage a forehead of the subject's head on the first side of the subject's head,
the second head restraint includes a second forehead flange configured to selectively engage the subject's forehead on the second side of the subject's head,
the first head restraint includes a first crown flange configured to selectively engage a crown of the subject's head on the first side of the subject's head, and
the second head restraint includes a second crown flange configured to selectively engage the subject's crown on the second side of the subject's head, the head restraint assembly further comprising:
a forehead strap extending between the first and second forehead flanges and configured for selectively tightening the forehead flanges against the subject's forehead; and
and a crown strap extending between the first and second crown flanges and configured for selectively tightening the crown flanges against the subject's crown.

6. A restraint assembly for helping to restrain a subject, comprising:
the head restraint assembly of claim 1; and
a body stabilizer configured to receive a portion of the subject's body below the subject's head, the body stabilizer being removably attached to the head restraint assembly.

7. The restraint assembly of claim 6, further comprising at least one winglet configured to receive an arm of the subject, the at least one winglet being attached to the body stabilizer and pivotable relative to the body stabilizer.

8. The restraint assembly of claim 6, further comprising a foot platform configured to receive at least the subject's feet and be removably attached to the body stabilizer.

9. The restraint assembly of claim 6, further comprising an abdominal sling having a central sling portion configured to selectively engage the subject's abdominal wall skin, the abdominal sling having at least one sling strap extending from the central sling portion and configured to be selectively attached to the body stabilizer,
wherein the abdominal sling is configured such that, when the central device portion engages the subject's abdominal wall skin and the at least one sling strap is attached to the body stabilizer, the central sling portion exerts a force on the subject's abdominal wall skin to urge the subject's abdominal wall soft tissue cephalad.

10. The restraint assembly of claim 6, wherein the body stabilizer comprises separate first and second body stabilizer portions that are selectively coupled to one another via a hinge mechanism, at least one of the first and second body stabilizer portions being pivotable relative to the other of the first and second body stabilizer portions between an unfolded and folded conditions via the hinge mechanism.

11. The head restraint assembly of claim 1, further comprising a quick release mechanism having a holding position in which the first and second head restraints are substantially prevented from detaching from the restraint platform, and a release position in which each of the first and second head restraints are detachable from the restraint platform.

12. A head restraint assembly, comprising:
a restraint platform configured to receive a head of a subject;
a first head restraint attached to the restraint platform and configured to selectively engage a first side of the subject's head;
a second head restraint attached to the restraint platform and configured to selectively engage a second side of the subject's head;
a first articulating assembly having a first portion attached to the first head restraint and a second portion connected to the restraint platform, the first portion of the first articulating assembly being selectively articulatable relative to the second portion of the first articulating assembly and thereby providing the first head restraint with at least three degrees of freedom of movement for the first head restraint to conform to the first side of the subject's head; and
a second articulating assembly having a first portion attached to the second head restraint and a second portion connected to the restraint platform, the first portion of the second articulating assembly being selectively articulatable relative to the second portion of the second articulating assembly and thereby providing the second head restraint with at least three degrees of freedom of movement for the second head restraint to conform to the second side of the subject's head.

13. The head restraint assembly of claim 12, wherein
the first articulating assembly comprises a first ball-and-socket assembly having a first ball portion attached to one of the first head restraint and a first ball-and-socket base and a first socket portion on the other one of the first head restraint and the first ball-and-socket base, the first ball portion being selectively received in the first socket portion and thereby providing the first head restraint with at least three degrees of freedom of movement for the first head restraint to conform to the first side of the subject's head, the first ball-and-socket base being connected to the restraint platform, and
the second articulating assembly comprises a second ball-and-socket assembly having a second ball portion attached to one of the second head restraint and a second ball-and-socket base and a second socket portion on the other one of the second head restraint and the second ball-and-socket base, the second ball portion being selectively received in the second socket portion and thereby providing the second head restraint with at least three degrees of freedom of movement for the second head restraint to conform to the second side of the subject's head, the second ball-and-socket base being connected to the restraint platform.

14. The head restraint assembly of claim 13, wherein
the first ball-and-socket assembly has a first ball inhibiting mechanism for selectively engaging the first ball portion to inhibit movement of the first ball portion relative to the first ball-and-socket base, the first head restraint being retained in a fixed position as a result of the first ball inhibiting mechanism inhibiting the movement of the first ball portion, and
the second ball-and-socket assembly has a second ball inhibiting mechanism for selectively engaging the second ball portion to inhibit movement of the second ball portion relative to the second ball-and-socket base, the second head restraint being retained in a fixed position as a result of the second ball inhibiting mechanism inhibiting the movement of the second ball portion.

15. The head restraint assembly of claim 12, further comprising:
a first sliding member configured to be selectively slid laterally along the restraint platform, the first sliding member being fixed to the first ball-and-socket base, selective lateral movement of the first sliding member responsively causing the first head restraint to move laterally relative to the restraint platform; and
a second sliding member configured to be selectively slid laterally along the restraint platform, the second sliding member being fixed to the second ball-and-socket base, selective lateral movement of the second sliding member responsively causing the second head restraint to move laterally relative to the restraint platform.

16. The head restraint assembly of claim 12, wherein
the first head restraint includes a first cheekbone flange configured to selectively engage a cheekbone region of the subject's head on the first side of the subject's head, and
the second head restraint includes a second cheekbone flange configured to selectively engage a cheekbone region of the subject's head on the second side of the subject's head, the head restraint assembly further comprising:
a cheekbone strap extending between the first and second cheekbone flanges and configured for selectively tightening the cheekbone flanges against the subject's cheekbone regions, the cheekbone strap being configured to be spaced from the subject's head when the subject's head is restrained by the head restraint assembly.

17. A restraint assembly for helping to restrain a subject, comprising:
the head restraint assembly of claim 12; and
a body stabilizer configured to receive a portion of the subject's body below the subject's head, the body stabilizer being removably attached to the head restraint assembly.

18. The restraint assembly of claim 17, further comprising at least one winglet configured to receive an arm of the subject, the at least one winglet being attached to the body stabilizer and pivotable relative to the body stabilizer.

19. The restraint assembly of claim 17, further comprising a foot platform configured to receive at least the subject's feet and be removably attached to the body stabilizer.

20. The restraint assembly of claim 17, further comprising an abdominal sling having a central sling portion configured to selectively engage the subject's abdominal wall skin, the abdominal sling having at least one sling strap extending from the central sling portion,
wherein the abdominal sling is configured such that when the central sling portion engages the subject's abdominal wall skin and the at least one sling strap is tensioned, the central sling portion exerts a force on the subject's abdominal wall skin to urge the subject's abdominal wall soft tissue cephalad.

21. The restraint assembly of claim 17, wherein the body stabilizer comprises separate first and second body stabilizer portions that are selectively coupled to one another via a hinge mechanism, at least one of the first and second body stabilizer portions being pivotable relative to the other of the first and second body stabilizer portions between an unfolded and folded conditions via the hinge mechanism.

22. The head restraint assembly of claim 12, further comprising a quick release mechanism having a holding position in which the first and second head restraints and the first and second articulating assemblies are substantially prevented from detaching from the restraint platform, and a release position in which the first and second head restraints and the first and second articulating assemblies are detachable from the restraint platform.

23. A body stabilizer assembly for helping to restrain a subject, comprising:
a body stabilizer configured to receive a portion of the subject, the body stabilizer including at least one stabilizer aperture longitudinally positioned along a first lateral stabilizer side of the body stabilizer and at least one stabilizer aperture longitudinally positioned along an opposite second lateral stabilizer side of the body stabilizer, each stabilizer aperture extending transversely through the body stabilizer;
a plurality of strap connectors, each strap connector being at least partially positioned in an associated stabilizer aperture and selectively longitudinally moveable along the associated stabilizer aperture; and
at least one restraining strap configured to be selectively attached to the body stabilizer via the strap connectors and selectively prevent the egress of the subject from the body stabilizer, each end of the at least one restraining strap being selectively connected to an associated strap connector;
wherein via the strap connectors, the at least one restraining strap is selectively longitudinally moveable relative to the body stabilizer to correspond to the subject's anatomy.

24. The body stabilizer assembly of claim 23, further comprising at least one winglet configured to receive an arm of the subject, the at least one winglet being attached to the body stabilizer and selectively pivotable relative to the body stabilizer.

25. A restraint assembly for helping to restrain a subject, comprising:
the body stabilizer assembly of claim 23; and
a head restraint assembly configured to receive a portion of the subject's head, the head restraint assembly being removably attached to the body stabilizer.

26. The restraint assembly of claim 25, further comprising an abdominal sling having a central sling portion configured to selectively engage the subject's abdominal wall skin, the abdominal sling having at least one sling strap extending from the central sling portion and configured to be selectively attached to the body stabilizer,
wherein the abdominal sling is configured such that, when the central device portion engages the subject's abdominal wall skin and the at least one sling strap is attached to the body stabilizer, the central sling portion exerts a force on the subject's abdominal wall skin to urge the subject's abdominal wall soft tissue cephalad.

27. The restraint assembly of claim 25, wherein the head restraint assembly comprises:
a restraint platform configured to receive a head of a subject;
a first head restraint attached to the restraint platform and configured to selectively engage a first side of the subject's head;
a second head restraint attached to the restraint platform and configured to selectively engage a second side of the subject's head;
a first articulating assembly having a first portion attached to the first head restraint and a second portion connected to the restraint platform, the first portion of the first articulating assembly being selectively articulatable relative to the second portion of the first articulating assembly and thereby providing the first head restraint with at least three degrees of freedom of movement for the first head restraint to conform to the first side of the subject's head; and
a second articulating assembly having a first portion attached to the second head restraint and a second portion connected to the restraint platform, the first portion of the second articulating assembly being selectively articulatable relative to the second portion of the second articulating assembly and thereby providing the second head restraint with at least three degrees of freedom of movement for the second head restraint to conform to the second side of the subject's head.

28. The restraint assembly of claim 25, wherein the head restraint assembly comprises:
a restraint platform configured to receive a head of a subject;
a first head restraint attached to the restraint platform and configured to selectively engage a first side of the subject's head, the first head restraint having a first cheekbone flange configured to selectively engage a cheekbone region of the subject's head on the first side of the subject's head;
a second head restraint attached to the restraint platform and configured to selectively engage a second side of the subject's head, the second head restraint having a second cheekbone flange configured to selectively engage a cheekbone region of the subject's head on the second side of the subject's head; and
a cheekbone strap extending between the first and second cheekbone flanges and configured for selectively tightening the cheekbone flanges against the subject's cheekbone regions, the cheekbone strap being configured to be spaced from the subject's head when the subject's head is restrained by the head restraint assembly.

29. The body stabilizer assembly of claim 23, further comprising a tray assembly selectively attached to the body stabilizer, the tray assembly including a at least one tray connector selectively connected to the body stabilizer, at least one tray leg selectively connected to the tray connector, and a tray table selectively connected to the at least one tray leg.

30. The restraint assembly of claim 23, wherein the body stabilizer comprises separate first and second body stabilizer portions that are selectively coupled to one another via a hinge mechanism, at least one of the first and second body stabilizer portions being pivotable relative to the other of the first and second body stabilizer portions between an unfolded and folded conditions via the hinge mechanism.
